(19) United States
(12) Patent Application Publication
(10) Pub. No.: US 2009/0252754 A1
(43) Pub. Date: Oct. 8, 2009

(54) Vaccines containing the hiv tat protein as an adjuvant for the enhancement of cytotoxic t-cell responses

(76) Inventors: Antonella Caputo, San Nicolo (IT); Riccardo Gavioli, Ferrara (IT); Barbara Ensoli, Rome (IT)

Correspondence Address: FOLEY & LARDNER LLP, P.O. BOX 80278, SAN DIEGO, CA, 92138-0278, US

(21) Appl. No.: 10/574,751

(22) PCT Filed: Oct. 11, 2004
(86) PCT No.: PCT/EP04/11950

§ 371 Date: Feb. 2, 2007

(30) Foreign Application Priority Data
Oct. 10, 2003 (GB) 0322840.9

(51) Int. Cl. A61K 39/21; C07K 14/155

(52) U.S. Cl. 424/188.1; 530/324

(57) ABSTRACT

Tat, when used in a vaccine, causes MHC-I to expose subdominant epitopes present within an antigen, thereby enabling an optimal immune response to be generated, within an individual, against the antigen and variants of the antigen, such as might be encountered with HIV or influenza viruses.

### FIELD OF THE INVENTION

The present invention relates to vaccines comprising Tat, biologically active derivatives thereof or precursors therefor, including nucleic acids encoding such, as well as to methods for vaccination comprising the use of such vaccines.

### BACKGROUND OF THE INVENTION

The Tat protein of HIV-1 is produced very early upon virus entry and is required for virus replication and infectivity. Recently, we have shown that biologically active Tat is very efficiently taken up by dendritic cells and activates them, increasing Th-1 type responses against heterologous antigens. In addition, Tat-based vaccines in monkeys have been shown to be safe, and to induce protective immunity which correlates with the generation of Th-1 type immune responses.

Tat is a regulatory protein of HIV-1 and is produced very early after infection. It is essential for HIV-1 gene expression, replication, and infectivity. During acute infection of T cells by HIV-1, Tat is released in the extracellular milieu in a biologically active form in the absence of cell death or permeability changes^{1,2}. Extracellular Tat is taken-up by neighbour cells where it modulates cellular functions, depending on the concentration, oxidation state, and cell type.

In EP-A-1279404, we show that biologically active monomeric Tat protein is very efficiently taken-up by monocyte-derived dendritic cells (DC) and that, after internalisation, it induces DC maturation and augments allogeneic and antigen-specific presentation by DC, increasing Th-1 responses against recall antigens³. Fanales-Belasio et al. (Journal of Immunology 2002, vol 168 (1), pp. 197-206) also discloses the ability of Tat to augment presentation.

In addition, studies in mice and monkeys have shown that Tat-based vaccines are safe and induce protective immunity against pathogenic virus challenge that correlates with Th-1 type immune responses and cytotoxic T cells^{4,5}.

Cytotoxic lymphocytes (CTLs) play an essential role in the control of intracellular pathogens, including HIV, suggesting that vaccines eliciting optimal CTL responses have applications for the prevention and/or for the control of virus-associated diseases and tumours.

CTLs recognise peptide epitopes expressed at the surface of target cells in association with MHC class I molecules⁶. The epitope is generated in the cytosol by degradation of the antigen, from where it is transported into the endoplasmic reticulum, where it associates with newly synthesised class I molecules. Often, CTL responses are directed to a single immunodominant peptide out of a larger number of potential epitopes within the same antigen. This phenomenon, known as immunodominance, is still poorly understood. However, the generation and presentation of peptides, the availability of responsive T cells, and little understood immunoregulatory effects can all influence the activation of an efficient immune response to a particular epitope.

The major enzymatic activity responsible for the generation of class I-associated peptides is the proteasome, a large multicatalytic protease that is essential for the degradation of intracellular proteins and the maintenance of cell viability^{7,8}.

Proteasomes consist of a 20S catalytic core arranged as four heptameric rings. The two outer rings contain structural α-subunits (α1-α7), while the inner rings contain β subunits (β1-β7), three of which (β1, β2, β5) exert catalytic activity through a nucleophilic attack on the peptide bond by the N-terminal threonine⁹. Biochemical studies on the specificities of the proteasome reveal three distinct proteolytic components, which are involved in chymotryptic, tryptic and post-acidic (also called caspase-like) hydrolysing activities. Analysis of the contribution of the individual β-subunits has demonstrated a clear correlation between the individual subunits and the cleavage after preferred amino acids¹⁰. When cells are exposed to IFN-γ, the three catalytic β-subunits are substituted by LMP2, LMP7 and MECL1 (also referred as LMP10). These subunits are also expressed in a constitutive manner in specific cell types such as dendritic cells and B cells^{11,12}, and their incorporation in the proteasome alters its activity and enhances the production of certain peptides¹³.

Proteasomes equipped with LMP2, LMP7 and MECL1 have been called immunoproteasomes, as distinct from the constitutively expressed standard proteasomes. The catalytic activity of immunoproteasomes is characterised by a reduced cleavage after acidic amino acids and an increased cleavage after hydrophobic and basic residues, the most frequent residues found at the COOH terminus of MHC class I binding peptides¹⁴. It has been demonstrated that proteasomes generate the exact COOH terminus of MHC class I binding peptides, whereas the NH₂-terminal cleavage is not always as precise and that aminopeptidases located in the endoplasmic reticulum may cut the NH₂ extensions to generate the correct peptide epitope¹⁵⁻¹⁸.

For full and regulated proteasome function, the 20S proteasome core must assemble with other proteasome components, such as the 19S cap complex, to form the 26S proteasome which is able to degrade ubiquitin-conjugated proteins or/and the PA28 proteasome regulator to form the PA28-proteasome complex. The association of PA28 with the 20S proteasome seems to favour the generation of immunogenic peptides¹⁹. The generation of immunogenic peptides is a critical step in the activation of epitope-specific CTL responses. Indeed, there is evidence demonstrating that proteasome-mediated proteolysis contributes to the hierarchy of epitopes presented by MHC class I molecules. Subdominant T cell epitopes, in contrast to the immunodominant epitopes, are generated with less efficiency or are destroyed at cleavage sites located within the epitope^{20,21}.

Cafaro et al (Nature Medicine (1999), Vol 5, pp. 643-650), shows that the use of biologically active Tat in an HIV-1 vaccine for monkeys is safe and elicits a broad (both cellular and humoral) but specific immune response and reduces infection with SIV.

WO 00/43037 discloses that Tat and Nef are chemotactic agents for CD4+ cells and that vaccine efficacy may be boosted by the recruitment of CD4+ cells to the site of vaccine injection, when said vaccine is supplemented with Tat and Nef.

WO 02/019968 discloses a co-expression DNA vaccine (CED) that displays immunogenic properties. In particular, a vaccine encoding both an antigen and Tat is disclosed, the antigen benefiting from Tat-mediated immune deviation or immunomodulation/immunoregulation.

We have now, surprisingly, found that the Tat protein induces modifications of the subunit composition of immunoproteasomes in cells either expressing Tat or exposed to exogenous, biologically active Tat protein. In particular, Tat up-regulates the expression of the IFN-γ inducible catalytic subunits LMP7 and MECL1, but down-modulates LMP2. These changes correlate with an increase of all three of the major proteolytic activities of the proteasome. Proteasomes play a key role in the production of MHC class I binding peptides, and we found that Tat decreases the generation and presentation of immunodominant epitopes, while increasing the generation and presentation of subdominant T cell epitopes.

We have also found that modulation of proteosome subunit composition may be achieved by not only wild type Tat, but also by mutated Tat and Tat-derived peptides.

### SUMMARY OF THE INVENTION

Thus, in a first aspect, the present invention provides the use of Tat, a biologically active equivalent, or a precursor therefor, in the preparation of a vaccine suitable to elicit an immune response against an antigenic substance having a plurality of epitopes, the epitopes including both immunodominant and sub-dominant epitopes, the vaccine comprising at least a part of the antigenic substance encoding or comprising a sub-dominant epitope thereof.

Thus, Tat, when used in a vaccine, causes MHC-I to expose subdominant epitopes of a variable antigen, thereby enabling a persistent immune response to be generated within an individual against variants of the antigen, such as might be encountered with HIV or influenza viruses.

In a preferred embodiment, there is provided the use of Tat, a biologically active equivalent thereof or a precursor therefor, in the preparation of a vaccine suitable to elicit an immune response against a plurality of strains of an infectious organism, the vaccine comprising antigenic material from at least one strain of the organism, said material encoding or comprising a subdominant epitope.

It will be understood that a “precursor” includes any suitable material leading to the presence of Tat in the patient in a manner suitable to act as an adjuvant. This may include peptide precursors, such as fusion proteins, including fusions with signal peptides, which may be cleaved to yield active Tat, or which may be active without cleavage, or may include nucleic acid sequences in a form suitable to be expressed in situ.

Preferably, the Tat used is the wild type Tat shown in SEQ ID NO 284 or is a mutant and/or fragment thereof.

In one aspect, Tat is mutated. Any number of mutations, whether by substitution, deletion or insertion is envisaged, provided that the mutant is capable of increasing the number of subdominant epitopes presented, preferably by modulation of the proteosome subunits, as described above.

Preferably, the mutant has 90% homology to wild type Tat, according to SEQ ID NO 284, preferably 95% and more preferably 99% homology or sequence identity, as measured by known methods, such as the BLAST program.

In a particularly preferred embodiment, Tat is mutated at position 22. Preferably, the cysteine residue present in the wild type Tat at this position is substituted, preferably by glycine. Other suitable amino acids may also be used, such as alanine or any other non-polar amino acid.

In one embodiment, it is preferred that a fragment of Tat is used in the present invention. Any length peptide may be employed, provided that the above effect is seen. It is particularly preferred, however, that the fragment comprises or encodes at least amino acid numbers 47-86 of SEQ ID NO 284, which are given separately as SEQ ID NO 285. Preferably, the precursor is a polynucleotide, preferably DNA or RNA, encoding at least the above amino acids. It is also preferred that the fragment is a polypeptide comprising these amino acids. More preferably, the polypeptide consists of amino acids 47-86 of SEQ ID NO 284.

Preferably, however, the fragment comprises or encodes for up to: 15, 20, 30, 40, 50, 60, 70, 80, 90, 100, 150 or 200 or more amino acids.

Preferably, Tat may be expressed in situ, preferably in a target cell. The cell may be targeted, preferably, in vitro or more preferably, in vivo. Alternatively a benign, transformed organism may be introduced into the patient, the organism preferably expressing both Tat and the antigen against which it is desired to raise an immune response, but at least expressing Tat. The organism is suitably a virus or bacterium, and may be an attenuated form of an organism against which it is desired to stimulate an immune response.

Preferably, Tat is expressed in situ under the control of an inducible promoter, such that expression of Tat in the target cell can be induced by the user at or near the same time as administration or expression of the antigen. Suitable inducible promoters are well known but include those activated by physical means such as the heat shock promoter, although this is not generally preferred, or those activated by chemical such as IPTG or Tetracycline (Tet). The Tet promoter system is particularly preferred as it allows both on/off control of expression and control of the level of expression.

For Tat to be expressed in the target cell, it is preferable that the vaccine comprises an expression sequence. This sequence, element or vector is capable of expressing Tat in said cell and may be a viral vector, preferably attenuated, preferably an adenoviral vector capsid that can induce expression of Tat in the target cell. Other methods of inducing gene expression in target cells are known in the art and are also mentioned below.

It is, therefore, also preferred that the antigen is administered with a factor that controls or induces expression of Tat from an inducible promoter.

Alternatively, it is preferred that the antigen is expressed by administering a polynucleotide sequence encoding the antigen and that either a further polynucleotide sequence encoding Tat or a polynucleotide encoding a factor capable of inducing expression of Tat, is also provided, preferably substantially contemporaneously.

Expression in situ can be achieved by known methods of gene expression, such as the use of vectors, preferably viral vectors, that lead to expression of foreign DNA or RNA in a host. Preferably, polynucleotides encoding Tat are delivered and expressed by adenoviral or attenuated HIV systems. Alternatively, the polynucleotides can be delivered and expressed by methods such as the use of so-called “gene-guns.” Thus, it is preferred that Tat is endogenously expressed by the patient or vaccinee.

It will also be understood that where reference to Tat is made in the present application, it is intended to include mutants and fragments thereof, as also discussed herein, unless otherwise apparent to the skilled person. For instance, Tat may be wild type Tat or a shortened fragment of the Tat polypeptide sequence, or may be a tat mutant.

It is also preferred that Tat is exogenously produced and provided as a peptide. Preferably, Tat is administered as a precursor that may be cleaved in vivo to provide active Tat.

The patient or vaccinee is preferably a mammal, preferably an ape or monkey and mostly preferably a human. In an alternative embodiment, however, it is also preferred that the method, use or vaccine is not applied or administered to humans.

In a further aspect, the present invention also provides a vaccine or method for modulating proteosome subunit composition, preferably down-regulating particular subunit or subunits, preferably the LMP2 subunit.

In a still further aspect, the present invention also provides vaccines for eliciting an immune response against an antigenic substance having a plurality of epitopes, or for eliciting an immune response against a plurality of strains of an infectious organism, as discussed above. In one embodiment, the vaccine preferably comprises Tat, a biologically active equivalent, or a precursor therefor, the epitopes including both immunodominant and sub-dominant epitopes, and at least a part of the antigenic substance encoding or comprising a sub-dominant epitope thereof.

In a further embodiment, the vaccine preferably comprises Tat, a biologically active equivalent, thereof or a precursor therefore, and antigenic material from at least one strain of the organism, said material encoding or comprising a subdominant epitope.

As discussed above, Tat is preferably that shown in SEQ ID NO. 284. Furthermore, it is preferred that Tat and the antigen are provided as proteins or peptides.

The present invention also provides for the use of these vaccines, preferably to stimulate cross-strain immunity preferably in the treatment of disease, preferably HIV or influenza.

Preferably, the vaccine comprises a suitable vehicle for delivery of Tat and the antigen. Such vehicles are well known in the art.

### DETAILED DESCRIPTION OF THE INVENTION

While a major application of the present invention is in the fight against viruses well-known to generate escape mutants, it is equally important for use against cancer and immunomediated diseases, where the ability to target sub-dominant epitopes is a significant advantage.

In addition, it will be understood that Tat may be used with a vaccine of the present invention to enable the identification of subdominant epitopes of any antigens, especially from disease forms. Identification may effectively take the form of subtractive analysis. An example of this might be to take identical animals, immunise one against a tumour using standard vaccine, and another with a similar vaccine containing Tat, and then identifying what CTL epitopes the second animal had extra, by comparison with the first. Epitopes identified in this manner could be identified and isolated and used in vaccination programs, or in screening.

Subdominant epitopes are commonly found, not only in infectious organisms, but also in tumour and immunomediated disease antigens. Without being bound by theory, the presence of Tat appears to expose a greater number of regions of such antigens, thereby generating a more potent immune response against subdominant epitopes.

What is especially surprising is that it has been found that the use of Tat actually results in a reduction of CTL responses to immunodominant epitopes, despite the fact that these epitopes are still present and efficient. Indeed, the responses to these epitopes are reduced, while the response to subdominant epitopes becomes highly significant. It is particularly advantageous that the reduction of CTL responses against immunodominant epitopes is useful to avoid or reduce the formation of escape mutants.

In general, the effect of Tat appears to be to “equilibrate” CTL responses to the epitopes of an antigen, favouring a broader immunodominant/subdominant epitope-specific set of CTL responses to any given antigen. In addition, as stated above, the decrease of immunogenicity of immunodominant CTL epitopes may avoid escape mutants.

Previous vaccine studies in animals with Tat have shown the ability of this protein to stimulate an immune response, with the Tat protein acting both as an antigen and as an adjuvant. However, none of the studies, with Tat employed as an adjuvant or co-antigen, have shown or suggested the unexpected, unpredictable and striking ability of TAT to stimulate an immune response against the subdominant epitopes as demonstrated by the present invention. Essentially, no one would expect that Tat or, indeed, any other protein, to so significantly alter epitope generation and presentation.

Instead, we have now found that Tat can be used with a broad range of antigenic materials and that strong immune responses can be stimulated and observed against subdominant epitopes which rarely generate a response, or even which generate no response in most individuals.

Subdominant epitopes may either be observed on antigenic materials also comprising dominant epitopes, or may be comprised in molecules not associated with dominant epitopes. The location of the subdominant epitope is not important to the present invention, although it is generally preferred that it be available for recognition by CTLs during the life cycle of this organism, in order that the immune response generated be able to affect the course of the infection. Preferably, the infection will be controlled or eliminated by the immune response generated.

Escape mutations of dominant epitopes are very common, and have been observed in most disease organisms. For example, influenza and HIV are both associated with a number of strains where the dominant epitopes have mutated. Such mutation is widely believed to be a defence mechanism and, while the mutation has little or no effect on the virus, it is sufficient that an individual immune to one strain of the organism has no effective immunity against the organism carrying the new mutation.

By way of contrast, there has been no evolutionary pressure on subdominant epitopes to be able to mutate, so that there has also been no pressure on these epitopes to become dissociated from active functions in the organism. Accordingly, subdominant epitopes are substantially conserved and unable to mutate without crippling the organism.

Thus, by employing Tat in the vaccines of the present invention, it is possible to generate immunity against subdominant epitopes which are otherwise obscured by the dominant epitopes, and thereby to generate immunity against the CTL epitopes, both subdominant and immunodominant, within a given antigen, permitting an immune response to be generated against not only the strain of pathogen immunised against, but also a majority of, if not all, future and existing variants of the pathogen.

As used herein, the term “variant” includes all forms of the antigen that may be presented by the disease form in question, provided that the antigen in question is still presented by the disease form. It is not readily possible to define the term more closely, but it will be appreciated that escape mutations often mutate the immunodominant epitopes substantially, so that one form of variant might include those where only the immunodominant epitopes vary, but the remainder of the antigen remains 99% and preferably 100% unchanged.

Antigens of the present invention may be derived from a number of sources, including plants, parasites and fingi. However, it is preferred that the antigen or antigens are derived from bacteria, preferably Mycobacteria, preferably Mycobacterium tuberculosis, M. bovis, or M. africanum. The antigen may also, preferably, be derived from staphylococcal or bacilli bacteria.

It will be understood that the term derived from includes antigenic peptide fragments from an organism or virus or even the organism or virus itself, provided that an epitope is provided.

It is particularly preferred that the antigen is derived from viral sources, preferably herpes viruses or from the family of pox viridae, preferably from respiratory-disease causing viruses, especially Adenoviruses Picornaviruses, Rhinoviruses, Echoviruses and Coxsackieviruses, preferably those that are responsible for influenza.

Indeed about 30 to 50% of all colds are caused by one of the >100 serotypes of rhinoviruses. At any one time only a few viruses are prevalent. Often a single virus is responsible during outbreaks in relatively closed populations, such as in a school or barracks. However, new disease-causing strains rapidly evolve which immunised or tolerant individuals are not capable of reacting to rapidly. The present invention helps to overcome this by increasing the number of sub-dominant epitopes, which can often be more highly conserved.

Preferably, the antigen is derived from Acute Respiratory Syndrome viruses, such as those leading to SARS.

Even more preferred is that the Antigen is derived from, is a fragment of or comprises an Immunodeficiency virus, preferably SIV, but most preferably HIV. Various HIV antigens are known, including Gag, Pol, Rev and Env. Preferably, the antigen is derived from Gag or Env. Indeed, we have shown in the accompanying Examples that Tat is particularly useful against both Gag and Env as the number of epitopes Gag or Env recognised by a host immune systems is greater in the presence of Tat.

Preferably, the antigen is derived from a cancer or tumour, preferably, a bowel, stomach, lung, colon, or pancreatic tumour, or a melanoma.

It is also preferred that tat is useful in the treatment of immunomediated diseases, preferably allergies, asthma, bronchitis, autoimmune diseases, arthritis, gout and allied conditions, infections, gastroenteritis, dysentery, constipation, neoplasia or conditions associated with immunosuppression.

It is also preferred that Tat can be used a as co-antigen, that is, that Tat can be administered or expressed together with an antigen, Tat having the beneficial effect of increasing the number of epitopes, particularly sub-dominant epitopes of the antigen.

A further advantage of administering or expressing Tat, preferably with a further antigen, is that an immune response will also be raised against Tat itself and it is envisaged that this could lead to an immune response to both the antigen and Tat.

Preferably, the antigen is administered as a protein or peptide. As discussed above, the protein or peptide may be modified such that it is protected from digestion or breakdown, for instance by use of glycosylation, provided that the protection can later be removed at the appropriate site, for instance the bloodstream, for instance by blood-borne glycosylases or glycosylases administered to the blood.

Preferred routes of administration are discussed below, include oral, intravenous, intramuscular, or subcutaneous. Preferably, the antigen is provided in a form adapted for such delivery, and may be in the form of a tablet, pill, suppository or liquid suitable for injection, or it may be contained with polysaccharide spheres or particles or nanoparticles.

The vaccines of the present invention comprise Tat, a biologically active equivalent mutant or fragment thereof, thereof or a precursor therefor. As shown in the accompanying Examples, oxidised Tat has little or no effect, so that it is important to retain the biological activity of Tat. Within this requirement, it is possible to alter the Tat molecule, provided that the enhanced proteolytic activities of the immuno-proteasomes is conserved. This level of activity should be at least 30% of that shown in the accompanying Examples for each proteolytic activity. Preferably, the proteolytic activity should be at least 50% of the activity shown in the Examples, and preferably 80% and more preferably at least 90% of the activity shown in the Examples. For the avoidance of doubt, where more than one level of increased proteolytic activity is demonstrated in the accompanying Examples, then the above definition applies to the least of the listed activities, but may apply to any of the others, and preferably applies to the greatest activity.

Tat contains four domains. The acidic domain (amino acid residues 1 to 21) is important for interaction with cellular proteins. The cysteine rich region (amino acid residues 22 to 37) corresponds to the transactivation domain and is highly conserved among primary isolates. For example, replacing cysteine 22 with a glycine residue, leading to a so-called Tat22 mutant, abolishes the ability of Tat to transactivate the HIV-LTR. Likewise, the core domain (amino acid residues 38 to 48) is highly conserved, and simple substitution of lysine 41 with a threonine also incapacitates the transactivating ability of Tat on HIV-LTR. The fourth domain is the basic domain (amino acid residues 49 to 57), which is rich in arginine and lysine, and is responsible for the nuclear localisation of Tat, binding specifically to target RNA. This fourth domain is also responsible for binding extracellular Tat to heparin and heparansulphate proteoglycans. The carboxy terminal region is not necessary for LTR transactivation, but contains an arginine-glycine-aspartic acid sequence (RGD), common to extracellular matrix proteins; responsible for the interaction and binding of Tat to the integrin receptors α₅β₁ and αvβ₃.

Mutation of any of the domains or the carboxyl terminal is encompassed within the present invention, provided that the resultant biologically active Tat is still sufficient to stimulate the proteolytic activity of the immunoproteasomes as defined above.

In place of Tat, or a biologically active equivalent thereof, it is possible to use a nucleic acid sequence encoding either Tat or a biologically active equivalent thereof. In particular, the Tat, if not administered as part of the vaccine, may be expressed in situ, either by microbial systems in the vaccine, or as a result of administration of suitable expression sequences to the patient.

Likewise, the antigen comprised in the vaccine may also be presented in the form of a nucleic acid sequence encoding the antigen, or in the form of the original or a partially digested version of the original antigen, or a peptide. Although the subdominant epitope may be incorporated per se within the vaccine, this is not generally necessary when Tat is used, as Tat is capable of causing the presentation of subdominant epitopes by MHC-I.

It is convenient simply to incorporate antigenic material from the desired organism into the vaccine, as the unique activity of Tat is sufficient to decrease the immune response to the dominant epitope while substantially increasing the immune response to the subdominant epitope or epitopes. Although it is not essential to completely inactivate the infectious organism for the purposes of the vaccine, it is highly preferred, and this may be achieved by heat treatment or attenuation, for example. Further purification may be effected, if desired, such as by HPLC, ultrafiltration or centrifugation. Immunosorbent columns may also be used to separate ingredients.

The vaccines of the present invention may be used both for priming and boosting an immune response, and it is generally preferred that the composition of both the primary vaccine and booster is the same, although this is not necessary, provided that both the primary vaccine and the booster are to the same species of infectious organism, as the subdominant epitopes are conserved within the species.

Subsequent boosters may be applied as recommended by the skilled physician, and it is an advantage that it is not necessary to use the current virulent strain of an infectious organism to provide an effective vaccine.

The present invention further provides a method for providing an immune response against a plurality of strains of an infectious organism, comprising administering a vaccine comprising:

antigenic material from at least one strain of the organism, said material encoding or comprising a subdominant epitope; and
Tat, a biologically active equivalent thereof or a precursor therefor.

Preferably, Tat is as disclosed in SEQ ID NO. 284 or is a mutant and/or fragment thereof, as discussed elsewhere herein, and references to Tat and associated terms should be construed accordingly, in the absence of any indication to the contrary.

It is preferred that the infectious organism be a disease organism, and it is particularly preferred that the organism be a virus, although this is not necessary. Suitable sources of antigens are well known and are further discussed above.

Vaccines for use in the present invention may be provided in any suitable form and may be for administration by any suitable route. For example, vaccines of the invention may be provided intravenously, intramuscularly, intraperitoneally, subcutaneously, transdermally or in the form of eyedrops, or even as pessaries or suppositories.

Vaccines of the present invention may comprise any suitable ingredients in addition to the Tat and antigen ingredients, including, for example, stabilisers, buffers, saline, and isotonicity agents for injections, and any suitable ingredients, such as emulsifying agents and solid vehicles for applications such as pessaries and suppositories. Antibacterial and sterilising agents may also be employed, if desired.

In accompanying Example 1, we show that native HIV-1 Tat protein, an early product of HIV-infected cells, modifies the subunit composition and the activity of proteasomes. In particular, proteasomes in cells of B and T cell origin, either expressing endogenous Tat or exposed to a biologically active Tat protein, show up-regulation of LMP7 and MECL1 subunits and down-modulation of the LMP2 subunit. Strong down-regulation of the LMP2 subunit was shown to occur in splenocytes isolated from mice after treatment with native Tat but not with oxidised Tat protein, and selective down-regulation of LMP2 by viral gene products has been reported^{35,36}. It is known that the substitution of standard β-subunits with IFN-γ-inducible subunits alters the hydrolytic activity of proteasomes towards tri- and tetra-peptides, and the quality of the peptide products derived from polypeptides^{10,23-25}. We demonstrate here that changes in proteasome subunit composition induced by Tat result in the increase of all three major proteasome proteolytic activities^{10,23-25}.

Perturbation of the proteasome system by viral infection, cell transformation or pharmacological treatments is often a key event in the modulation of the immune response to pathogens³⁷⁻⁴⁴, since proteasomes play a pivotal role in the generation of the majority of antigenic peptides presented by MHC class I molecules⁸. In particular, immunoproteasomes are very efficient for the generation of specific CTL epitopes, and it has been shown that substitution of standard β-subunits with LMP2, LMP7 and MECL1 subunits improves the production of peptide antigens with the correct C termini for binding to MHC class I⁴⁵⁻⁴⁸. By way of contrast, there is evidence, both in humans and mice, that the presence of LMP2 may inhibit the presentation of specific peptide antigens^{12,49,50}.

We now show that the variations in proteolytic activity of proteasomes in Tat-expressing cells or in cells exposed to Tat protein correlate with a different presentation of EBV-derived epitopes, for example. In the accompanying Example, we show that Tat decreases the presentation of two immunodominant CTL epitopes (IVT and AVF) presented by HLA-A11 molecules, and increases the presentation of two subdominant epitopes (YLQ and CLG) presented by HLA-A2. HLA-A2-associated peptides present a valine at the C terminus, and it has been demonstrated that the β1 subunit, replaced by LMP2 in the immunoproteasomes, is responsible for cleavage beyond acidic residues and beyond residues with branched chains, such as valine^{10,51}.

It is, therefore, preferred that the present invention stimulates the down-regulation or replacement of LMP2 subunits, and preferably an up-regulation of β1 subunits in proteasomes. It is also preferred that the present invention stimulates an increase in the number of peptides cleaved at Valine. Also preferred is a vaccine or method for increasing the number of epitopes recognises, particularly sub-dominant epitopes.

Preferably, Tat, its equivalent mutant or fragment, or precursor, is capable of down-regulating levels of LMP2 in the intended recipient of the vaccine.

Proteasomes in Tat-expressing/treated cells present low levels of LMP2 and higher post-acidic activity, compared with untreated cells or with cells that do not express Tat, which may account for the greater enhancement in the generation and presentation of YLQ and CLG CTL epitopes that present a C-terminal valine^{10,51}. We also show that proteasomes from Tat-expressing cells are very efficient in the degradation of a CLG peptide precursor and can generate immunogenic peptide fragments therefrom, in contrast to proteasomes isolated from control cells.

A similar phenomenon was observed for an HLA-A2 presented epitope expressed in melanoma cells¹², suggesting that the presence of LMP2 may particularly affect the range of peptides presented by some HLA class I alleles, such as HLA-A2. This suggests that the presence of LMP2 is critical for the generation of CTL epitopes. Indeed, it has been demonstrated that influenza-specific CTL responses to the two most dominant determinants decrease in LMP2 knock-out mice, whereas responses to two subdominant epitopes are greatly enhanced⁵⁰. Similarly, we demonstrated that the Tat-dependent LMP2 down-modulation induces changes in the hierarchy of CTL responses directed to Ova-derived CTL epitopes.

What we have demonstrated, for the first time, is that Tat increases CTL responses directed to subdominant epitopes and decreases these directed to the immunodominant SII peptide.

This is achieved by modifying the catalytic subunit composition and activity of immunoproteasomes in B and T cells which either express Tat, or have been treated with biologically active exogenous Tat protein. This results in modulation of the in vitro CTL epitope hierarchy. In particular, both intracellularly expressed and exogenous native Tat protein increase the major proteolytic activities of the proteasome by up-regulating LMP7 and MECL1 subunits and by down-modulating the LMP2 subunit. This results in a more efficient generation and presentation of subdominant CTL epitopes

Decreasing the presentation of immunodominant epitopes, accompanied with an increase in the presentation of subdominant epitopes, is particularly beneficial for the elimination of virally infected cells, given that it is well established that immunodominant epitopes are very prone to mutation and to viral-escape, while subdominant epitopes are more stable while being capable of inducing protection⁵².

Thus, Tat protein is useful to drive the induction of MHC-I restricted immune responses, broadening the spectrum of the epitopes recognised and increasing the chances to prevent the appearance of viral escape.

As mentioned above, we have shown that the presence of Tat results in a more efficient generation and presentation of subdominant CTL epitopes. Since the amount of MHC-I/epitope complexes is crucial in determining the presence and the strength of epitope-specific CTL responses and to verify the biological relevance of these findings for vaccination strategies, we went on to evaluate epitope-specific CTL responses against ovalbumin in mice vaccinated with both Tat and ovalbumin.

Surprisingly, we also found that Tat decreases CTL responses directed to the immunodominant epitope while inducing those directed to subdominant and cryptic T-cell epitopes that were not present in mice vaccinated with ovalbumin alone.

This finding suggests that Tat favours the generation of CTL responses directed to “weak” CTL epitopes and could therefore be used as a tool to increase CTL responses to heterologous antigens.

In addition, we found that a mutated form of the HIV-1 Tat protein, carrying glycine instead of cysteine 22 (Tatcys22) (SEQ ID NO 286), like wild-type Tat, modifies the subunit composition of proteasomes. The Tatcys22 mutant, in contrast to wild-type Tat, has no effect on the transactivation of the HIV-1 LTR, and does not induce reactivation of latent infection. This is particularly advantageous as administration or expression of biologically inactive tat may be appropriate in some circumstances.

Thus, we have also shown that the Cys residue at position 22, although key to the function of wild type Tat, is not required for Tat's effect on proteosome subunit composition.

It is a particular advantage of the TatCys22 mutant (SEQ ID NO 286) that it shows an improved effect compared to wild type Tat. That is, the TatCys22 mutant has actually been shown to increase the number of subdominant epitopes processed and, thereby, presented.

Indeed, our results in Experiment 3 show that T cell responses induced by vaccination with Gag+Tatcys22 are directed to 11 different T cell epitopes, 7 more than mice immunised with Gag alone, and 4 more than mice immunized with Gag and wild-type Tat.

We also demonstrate that peptide 47-86, derived from the wild-type Tat protein, is sufficient to down-modulate the LMP2 subunit.

Therefore, mutated forms of Tat, or Tat-derived peptides, represent an important alternative to the use of wild-type Tat in vaccination strategies aimed at increasing epitope-specific T cell responses directed to heterologous antigens.

We exploited the effect of Tat (both wild-type Tat protein and mutant Tatcys22 protein) on T cell responses against structural HIV gene products in vivo. We showed that, surprisingly, Tat increases the number of CTL epitopes within HIV Gag and Env antigens.

Balb/C mice were immunised with HIV-1 Gag or Env protein antigens, either alone or in combination with wild-type Tat or mutant Tatcys22. We found that both wild-type Tat and mutated Tatcys22 increase the number of epitope-specific T cell responses against Gag and Env antigens. In particular, we demonstrated that mice vaccinated with Gag, in combination with wild-type Tat or with the mutant Tatcys22, responded to 7 or 11 T cell Gag-derived epitopes respectively, in contrast to mice vaccinated with Gag alone, which responded to 4 T cell Gag-derived epitopes. Similarly, mice vaccinated with Env, in combination with wild-type Tat or with the mutant Tatcys22 responded to 12 Env-derived pools of epitopes, in contrast to mice vaccinated with Env alone, which responded to 8 T cell Env-derived peptide pools.

Our results show that Tat is not only an antigen but also an adjuvant capable of increasing T cell responses against heterologous antigens. Therefore, the Tat protein, as well as mutant Tatcys22, represents an important tool in HIV-1 vaccine strategies aimed at broadening the spectrum of the epitopes recognized by T cells.

Thus, Tat is a useful tool for inducing epitope-specific CTL responses against HIV antigens and can be used as co-antigen for the development of new vaccination strategies against AIDS.

### DESCRIPTION OF THE DRAWINGS

In the following Examples reference is made to the accompanying Figures, in which:

FIG. 1 shows Tat DNA and RNA Analysis in Transduced MIN and MON LCL's

FIG. 1A: PCR analysis was performed on genomic DNA (200 ng) from transduced and not transduced MIN- and MON-LCL's, using Tat1 and Tat2 primers. pCV-tat plasmid DNA (0.1 ng) was amplified as positive control. Amplified product is 240 bp. Molecular weight marker (MW): GeneRuler 100 bp DNA Ladder (MBI Fermentas).

FIG. 1B: RT-PCR analysis was performed on cDNA from transduced and not transduced LCL's, using Tat1 and Tat2 primers. pCV-Tat plasmid DNA (0.1 ng) was amplified as positive control. Molecular weight marker (MW): GeneRuler 100 bp DNA Ladder (MBI Fermentas). Panel c: Northern blot analysis: total RNA (40 μg), purified from transduced and not transduced MIN- and MON-LCL's, was hybridised with ³²P-labeled Tat PCR product as probe. The positions of 28S and 18S rRNA's, as molecular size markers, are indicated.

FIG. 2 shows Expression of Proteasome Subunits in Cells Transduced with HIV-1 Tat Gene

Left panel: Equal amount of total proteins from cell lysates from MIN and MON LCL's transduced with pBabeP (MIN-0 and MON-0) or with pBabeP-Tat (MIN-Tat and MON-Tat) were fractionated by SDS-PAGE, transferred onto nitrocellulose filters, and probed with mAbs or polyclonal anti-sera specific for the α-2 subunit, PA28α, LMP2, LMP7 and MECL1. Right panel: The intensity of specific bands was measured by densitometry. Data are expressed as % increase in optical densities of Tat expressing cells relative to control cells. One representative experiment out of four performed is shown.

FIG. 3 shows Activity of Proteasomes Purified from Cells Transduced with the HIV-1 Tat Gene

Purified proteasomes from cell lysates of MIN and MON LCL's transduced with pBabeP (MIN-0 and MON-0) or with pBabeP-Tat (MIN-Tat and MON-Tat) were tested for chymotryptic-like, tryptic-like and post-acidic activities using Suc-LLVY-AMC, Boc-LRR-AMC and Ac-YVAD-AMC as substrates, respectively. Peptide substrates (100 μM) were incubated with 5 μg of purified proteasomes at 37° C. for 30 min. Data are expressed as arbitrary fluorescence units. One representative experiment out of three performed is shown.

FIG. 4 shows Expression of Proteasome Subunits in Cells Treated with the HIV-1 Tat Protein

Left panel: Equal amount of total proteins from cell lysates from MIN and MON LCL's treated with the indicated concentrations of the native Tat protein were fractionated by SDS-PAGE, transferred onto nitrocellulose filters, and probed with mAbs or polyclonal anti-sera specific for the α-2 subunit, LMP2, LMP7 and MECL1. Right panel: The intensity of specific bands was measured by densitometry. Data are expressed as % increase in optical densities of Tat expressing cells relative to control cells. One representative experiment out of three performed is shown.

FIG. 5 shows Expression of the LMP2 Subunit in Splenocytes Isolated from Mice Treated with Tat Protein

Mice were treated with native Tat protein (panel a) or with oxidized Tat (panel b) and after 3 i.m. treatments, splenocytes were isolated and lysed. Equal amount of total proteins from cell lysates were fractionated by SDS-PAGE, transferred onto nitrocellulose filters, and probed with an antibody specific for LMP2. The intensity of LMP2 bands was evaluated by densitometry and normalised to the correspondent expression of proteasomes evaluated with a polyclonal sera specific for α-subunits. Data are expressed as % increase in optical densities compared to the mean of LMP2 expression in control splenocytes from 6 untreated mice.

FIG. 6 shows CTL Killing of Cells Transduced with the HIV-1 Tat Gene

The HLA-A2, -A11 positive MN and MON LCL's transduced with pBabeP (MIN-0 and MON-0) or with pBabeP-Tat (MIN-Tat and MON-Tat) were used as target in cytotoxic assays of CTLs specific for the HLA-A11 presented, EBNA4-derived IVT and AVF epitopes, the HLA-A2-presented Lmp1-derived YLQ epitope, and the HLA-A2-presented Lmp2-derived CLG epitope, respectively. Results are expressed as % specific lysis. One representative experiment out of three performed is shown.

FIG. 7 shows CTL Killing of Cells Treated with Exogenous HIV-1 Tat Protein

The HLA-A2, -A11 positive MIN LCL's, treated or not with Tat, were used as target in cytotoxic assays of CTLs specific for the HLA-A11 presented, EBNA4-derived IVT and AVF epitopes, the HLA-A2-presented Lmp1-derived YLQ epitope, and the HLA-A2-presented Lmp2-derived CLG epitope. Results are expressed as % specific lysis. One representative experiment out of three performed is shown.

FIG. 8. In Vitro Degradation of a CLG Epitope Precursor by Proteasomes Purified from Tat-Expressing Cells.

Panel A: the CLG+5 peptide was incubated with proteasomes purified from MIN-Tat or from MIN-0 LCLs. The precursor degradation was followed at different time points and the degradation of CLG+5 was evaluated by HPLC analysis. Data are expressed as % degradation. The mean of the results from three independent experiments is shown.

Panel B: the digestion products obtained after 120 min of degradation were purified by HLPC, the indicated fractions were collected and tested by IFN-γ Elispot for their capacity to activate CLG-specific CTLs. Data are expressed as spot-forming cells (SFC) per 10⁶ cells. The mean of the results from three independent experiments, performed in triplicates, is shown.

FIG. 9 shows Ova-Specific CTL Responses in Mice Vaccinated with Ova and Tat Protein

Mice were vaccinated with Ova alone or with Ova and Tat protein. After 2 immunisations, fresh splenocytes were pooled and tested in cytotoxicity against EL4 cells pulsed with SII, KVV, or CFD peptides. Data are expressed as % specific lysis calculated by subtracting lysis of untreated EL4 cells (always below 10%). Mean of two independent experiments performed in triplicate.

FIG. 10 shows Expression of Proteasomes in Jurkat Cells Expressing the HIV-1 Tat Gene.

FIG. 10A: equal amounts of purified proteasomes (1 μg) from Jurkat cells transfected with the vector alone (JSL3-0), or with the tat gene (JSL3-Tat), were fractionated by SDS-PAGE, transferred onto nitrocellulose filters, and probed with mAbs or polyclonal anti-sera specific for α-2 subunit, LMP2, LMP7 and MECL1. One representative experiment out of the four performed is shown.

FIG. 10B: The intensity of specific bands was measured by densitometry. Data are expressed as % increase in optical densities of specific bands detected in proteasomes purified from Tat expressing cells, relative to proteasomes from control cells. Mean+/−SEM of three independent experiments is shown.

FIG. 11. Expression of Proteasome Subunits in Jurkat Cells Treated with the HIV-1 Tat Protein.

Jurkat cells were treated for 12 (FIG. 11A) or for 24 (FIG. 11B) hours at 37° C. with 0.01, 0.1 or 1 μg/ml of the native Tat protein. Equal amounts of proteasomes (1 μg) were fractionated by SDS-PAGE, transferred onto nitrocellulose filters, and probed with mAbs specific for the α-2 and LMP2 subunits. One representative experiment out of three performed is shown. The intensity of specific bands was measured by densitometry. Data are expressed in optical densities of specific bands detected in control cells (NT) and in Tat treated cells.

FIG. 12 Enzymatic Activity of Proteasomes in Jurkat Cells Treated or Untreated with 1 μg/ml of Tat for 24 Hours.

Proteasomes (2.5 μg) purified from cell lysates of the indicated cell lines were incubated for 30 min at 37° C. with Suc-LLVY-AMC, Boc-LRR-AMC and Ac-YVAD-AMC to evaluate chymotryptic-like, tryptic-like and post-acidic activities, respectively. Data are expressed as arbitrary fluorescence units.

FIG. 13. Expression of Proteasomes in Jurkat Cells Expressing Wild-Type or Mutated HIV-1 Tat Genes.

FIG. 13A: equal amounts of purified proteasomes (1 μg) from Jurkat cells transfected with the vector alone (Vect) or expressing wild-type Tat (Tat), mutant Tat22 (cys22 substituted with gly), mutant 37 (cys37 substituted with ser), or double mutant Tat22/37 were fractionated by SDS-PAGE, transferred onto nitrocellulose filters, and probed with α-2 subunit- and LMP2 subunit-specific mAbs. One representative experiment out of the four performed is shown.

FIG. 13B: the intensity of specific bands was measured by densitometry. Data are expressed as % increase in optical densities of specific bands detected in proteasomes purified from Tat expressing cells, relative to proteasomes from control cells.

FIG. 14. The Tat-Derived 47-86 Peptide is Sufficient to Down-Modulate the LMP2 Subunit.

Jurkat cells were treated for 24 with 0.1 μg/ml with Tat or with peptides 1-38, 21-58 and 47-86 covering the wild-type sequence of Tat. Equal amounts of proteins from total cell lysates were fractionated by SDS-PAGE, transferred onto nitrocellulose filters, and probed with α-2 subunit- and LMP2 subunit-specific mAbs. One representative experiment out of the three performed is shown.

FIG. 15. Ova-Specific CTL Responses in Mice Vaccinated with Ova Alone or Combined with the Tat Protein.

Mice were immunized with 25 μg of ovalbumin alone or in combination with 5 and 10 μg Tat protein. After 2 immunizations, fresh splenocytes were pooled and tested in cytotoxicity against EL4 cells pulsed with SII, KVV, or CFD peptides. Data are expressed as % specific lysis calculated by subtracting lysis of untreated EL4 cells (always below 10%). The mean of the results from three independent experiments, performed in triplicates, is shown.

FIG. 16. Tat Broadens the Immune Response Against Env.

Mice (n=5) were immunized subcutaneously with Tat, TatCys22 and Env proteins alone or in combination, as described in materials and methods. Splenocytes (pools of spleens) of immunized mice were stimulated with pools of Env peptides, and tested for IFNγ production in the presence of each pool, medium alone (negative control) or Concanavaline A (positive control). Results are expressed as the number of spot forming units (SFU)/10⁶ cells subtracted from the SFU/10⁶ cells of the negative controls, as described in Example 3. Responses ≧50 SFU/10⁶ cells are considered positive. Filled boxes mark reactive pools.

FIG. 17. Gag-Specific IFNγ T Cell Responses in Mice Vaccinated with Gag Alone or Combined with the Tat Protein.

Mice (n=5) were immunized with 5 μg of Gag alone or in combination with 5 μg Tat protein. After 3 immunizations, fresh splenocytes were pooled, stimulated with the indicated pools of Gag peptides and tested for IFNγ release by Elispot assay. Results are expressed as SFU/10⁶ cells subtracted from the SFU/10⁶ cells of the negative controls, as described in Example 3. Responses ≧50 SFU/10⁶ cells are considered positive.

FIG. 18. Gag-Specific IFNγ T Cell Responses in Mice Vaccinated with Gag Alone or Combined with the Tat Protein.

Mice (n=75) were immunized with 5 μg of Gag alone or in combination with 5 μg Tat protein. After 3 immunizations, fresh splenocytes were pooled, stimulated with the indicated peptides of Gag peptides and tested for IFNγ release by Elispot assay. Results are expressed as SFU/10⁶ cells subtracted from the SFU/10⁶ cells of the negative controls, as described in Example 3. Responses ≧50 SFU/10⁶ cells are considered positive.

FIG. 19. Gag-Specific IFNγ T Cell Responses in Mice Vaccinated with Gag Alone or Combined with the Tatcys22 Protein.

Mice (n=5) were immunized with 5 μg of Gag alone or in combination with 5 μg Tatcys22 protein. After 3 immunizations, fresh splenocytes were pooled, stimulated with the indicated pools of Gag peptides and tested for IFNγ release by Elispot assay. Results are expressed as SFU/10⁶ cells subtracted from the SFU/10⁶ cells of the negative controls, as described in Example 3. Responses ≧50 SFU/10⁶ cells are considered positive.

FIG. 20. Gag-Specific IFNγ T Cell Responses in Mice Vaccinated with Gag Alone or Combined with the Tatcys22 Protein.

Mice (n=5) were immunized with 5 μg of Gag alone or in combination with 5 μg Tatcys22 protein. After 3 immunizations, fresh splenocytes were pooled, stimulated with the indicated peptides of Gag peptides and tested for IFNγ release by Elispot assay. Results are expressed as SFU/10⁶ cells subtracted from the SFU/10⁶ cells of the negative controls, as described in Example 3. Responses ≧50 SFU/10⁶ cells are considered positive.

FIG. 21. Peptide Matrix Setup for HIV-1 Env Peptides

Pools 1-7 and pools 12-30 were designed so that 2 independent pools contain one peptide in common.

Pool 1, contains Env 1-Env 19+Env 8771, 8772, 8773
Pool 2, contains Env 20-Env 38+Env 8789, 8790, 8791
Pool 3, contains Env 39-Env 57+Env 8805, 8806
Pool 4 contains Env 58-Env76+Env 8822

FIG. 22. Peptide Matrix Setup for HIV-1 Gag Peptides

Shows matrix used for use with the Gag peptides in Example 3.

The invention is not to be limited by what has been particularly shown and described, except as indicated by the appended claims. Indeed, while the invention will now be illustrated in connection in connection with the following Examples, it will be understood that it is not intended to limit the invention to these particular embodiments. On the contrary, it is intended to cover all alternatives modifications and equivalents, as may be included within the scope of the invention as defined by the appended claims.

### EXAMPLE 1

The following methods were used in this and the following Examples.

### Cells

PG13 murine amphotropic packaging cell line⁵⁴ was cultured in DMEM supplemented with 10% FCS. Jurkat T cell transfectants (pRPneo-c and pRPneo-c-Tat)²² were cultured in RPMI 1640 medium, supplemented with 10% FCS and 800 μg/ml neomycin (Sigma). Lymphoblastoid cell lines (LCL) were established by in vitro infection of normal B-lymphocytes from healthy donors with the B95.8 strain of EBV. LCL's were cultured in RPMI 1640 medium supplemented with 10% FCS.

### Plasmids

HIV-1 Tat cDNA sequence was amplified by PCR from pGEM-3-Tat plasmid²² using primers Tat A: 5′-GGGGAATTCATGGAGCCAGTAGAT-3′ (forward) (SEQ ID NO 271) and Tat B: 5′-CAAGAATTCCTATTCCTTCGGGCC-3′ (reverse) (SEQ ID NO 272) (annealing temperature 57° C.). The purified PCR product was sequenced and cloned into the EcoRI site of pBabePuro vector to generate pBabePuro-Tat⁵⁵.

### Packaging Cell Lines

The pBabePuro and pBabePuro-Tat vectors were transfected into PG13 packaging cell line by the calcium phosphate method⁵⁶. Transfected cells were cultured in selective medium containing 3 μg/ml of puromycin (Sigma). Production of recombinant retroviruses from selected cultures was tested by semiquantitative RT-PCR on cell-free DNase treated supernatants, using of primers PuroA/PuroB (PuroA: 5′-CGAGCTGCAAGAACTCTTCC-3′ (forward) (SEQ ID NO 273), PuroB: 5′-AGGCCTTCCATCTGTTGCTG-3′ (reverse) (SEQ ID NO 274); annealing temperature 57° C.) and TatA/TatB respectively.

### Cell Transduction

MIN and MON LCL's were transduced with pBabePuro-Tat (MIN-Tat and MON-Tat) or pBabePuro (MIN-0 and MON-0) recombinant retroviruses by co-cultivation with packaging cell lines using transwell-clear tissue culture membranes. Subconfluent PG13 pBabePuro and PG13 pBabePuro-Tat cells, grown in the lower chamber, were co-cultivated in the presence of 8 μg/ml polybrene (Sigma) with MIN or MON LCL's (3×10⁶/well) added to the upper chamber in 2.5 ml of RPMI 1640 medium supplemented with 10% FCS. After 48 hrs of co-cultivation, cells were harvested from the membranes and grown in culture medium containing puromycin (0.3 μg/ml) for 6 weeks. All cell lines were characterised by DNA-PCR, RT-PCR and Northern blot analysis.

### Characterisation of Transduced Cell Lines

Total DNA was extracted from 5×10⁶ cells with the NucleoSpin Blood kit (Macherey-Nagel), as specified by the manufacturer. For amplification of Puromycin and Tat genes, primers PuroA/PuroB were used, under the conditions described above, and Tat 1: 5′-gAAgCATCCAggAAgTCAgCC-3′ (SEQ ID NO 275) Tat 2: 5′-ACCTTCTTCTTCTATTCCggg-3′ (SEQ ID NO 276) (annealing temperature 55° C.).

RNA was extracted from cell-free supernatants of packaging cells, MIN and MON LCL's, MIN-0 and MON-0, MIN-Tat and MON-Tat cells (5×10⁶) with NucleoSpin RNA II (Macherey-Nagel), as specified by the manufacturer. Total RNA (1 μg) was incubated with 20 mM MgCl₂ and 500 IU/ml pancreatic DNase I (Boehringer Mannheim) at 37° C. for 1 hr and purified by phenol-chloroform. DNase digestion was repeated three times upon the addition of fresh DNase I.

RNA was reverse transcribed by using the random hexamer method with RT-PCR Systems (Promega) according to manufacturer's instructions. cDNA was tested by PCR using actin-specific primers:

(SEQ ID NO 277)

forward: 5′-TGACGGGGTCACCCACACTGTGCCCATCTA-3′;

(SEQ ID NO 278)

reverse: 5′-AGTCATAGTCCGCCTAGAAGCATTTGCGGT-3′;

annealing temperature 63° C.). PCR's for Puromycin and Tat genes were performed as described.

### Northern Blotting

Equal amounts of total RNA's (40 μg) were electrophoresed onto formaldehyde-agarose gel (1.5%) for 12 hours, transferred onto nylon membranes (Hybond N; Amersham) and hybridised with DNA probe. Probes were randomly labelled with [³²P] dCTP, using the Prime-It II kit (Stratagene).

### Western Blotting

Equal amounts of proteins were loaded on a 12% SDS-PAGE gel and electroblotted onto Protran nitrocellulose membranes (Schleicher & Schuell, Keene, Hampshire, USA). The blots were probed with antibodies specific for α2, LMP2, LMP7, MECL1, and PA28α subunits (Affinity, Exeter, UK) and developed by enhanced chemiluminescence (ECL, Amersham Pharmacia Biotech, Uppsala, SW).

### HIV-1 Tat Protein

HIV-1 Tat from the human T lymphotropic virus type IIIB isolate (subtype B) was expressed in E. Coli and purified by heparin-affinity chromatography and HPLC as a Good Laboratory Practice (GLP) manufactured product as described previously. The Tat protein was stored lyophilised at −80° C. to prevent oxidation and reconstituted in degassed buffer before use, as described². Different GLP lots of Tat were used with reproducible results and in all cases endotoxin concentration was below 0.05 EU/μg.

### Purification of Proteasomes

Cells (5×10⁷) were washed in cold PBS and resuspended in buffer containing 50 mM Tris-HCl pH 7.5, 5 mM MgCl₂, 1 mM dithiothreitol (DTT, Sigma), 2 mM ATP, 250 mM sucrose. Glass beads equivalent to the volume of the cellular suspension were added and cells were vortexed for 1 min at 4° C. Beads and cell debris were removed by 5 minutes centrifugation at 1000×g, followed by 20 minutes centrifugation at 10,000×g. Supernatants were ultracentrifuged for 1 hour at 100,000×g⁴⁴. Supernatants were loaded into an affinity column containing an agarose matrix derivatised with the MCP21 mAb specific for the α2 subunit of the proteasome (Affinity, Exeter, UK). The column was washed, eluted with 25 mM Tris-HCl pH 7.5 containing 2 M NaCl, and 0.5 ml fractions were collected. Fractions containing proteasomes were combined and dialysed against 25 mM Tris-HCl pH 7.5. Protein concentration was determined using BCA protocol (Pierce Chemical).

### Enzyme Assays

The fluorogenic substrates Suc-LLVY-AMC, Boc-LRR-AMC and Ac-YVAD-AMC were used to measure chymotrypsin-like, trypsin-like and post-acidic proteasome activities, respectively. Peptide substrates (100 μM) were incubated at 37° C. for 30 min with purified proteasomes in 75 μl of buffer containing 50 mM Tris-HCl pH 7.4, 5 mM MgCl₂, 500 μM EDTA pH 8.0, 1 mM dithiothreitol, and 2 mM ATP. Fluorescence was determined by a fluorimeter (Spectrafluor plus, Tecan, Salzburg, Austria) using an excitation of 360 nm and emission of 465 nm. Proteasome activity is expressed in arbitrary fluorescence units¹¹.

### Synthetic Peptides

All peptides were synthesised by solid phase methods⁵⁷. Crude deprotected peptides were purified by HPLC to >98% purity. Structure verification was performed by elemental and amino acid analysis and mass spectrometry. Peptide stocks were dissolved in DMSO at a concentration of 10⁻² M, kept at −20° C., and diluted in PBS before use.

### Digestion of Synthetic Substrates

The synthetic peptide CLGGLLTMVAGAVW (CLG+5) (SEQ ID NO 279) was dissolved in DMSO at a concentration of 20 μg/μl. 500 μg of synthetic peptide were incubated with 127 μg of purified proteasomes in 300 μl of buffer (25 mM Tris HCL pH 7.4, 5 mM MgCl₂, 500 μM EDTA pH 8.0, 1 mM DTT, 2 mM ATP) at 37° C. At the indicated time points, 60 μl of sample were collected and the reaction was stopped by adding 2 volumes of ethanol at 0° C.

Digestion mixtures were centrifuged at 5000 rpm for 5 minutes. 80 μl of supernatant were collected and peptide digests were separated by reverse-phase HPLC at the flow rate of 0.7 ml/min, as follows: linear gradient of 0-100% of solution B (acetonitrile 100% with 0.1% TFA) for 25 min, followed by linear gradient of 0-100% for solution A (water 100% with 0.1% TFA) for 5 min. The fractionation was simultaneously monitored at 210 and 280 nm. Fractions were collected every 30 seconds and stored at +4° C. and tested in ELISPOT.

### Generation of CTL Cultures

HLA A11-restricted EBV-specific CTL cultures reacting against the EBNA4-derived IVTDFSVIK (SEQ ID NO 280) (IVT) and AVFSRKSDAK (SEQ ID NO 281) (AVF) epitopes, corresponding to amino acid 416-424 and 399-40827, were obtained by stimulation of monocyte-depleted PBL's from the HLA-A11-positive EBV-seropositive donor MC with the autologous B95.8 virus-transformed LCL. HLA A2-restricted EBV-specific CTL cultures reacting against the Lmp2-derived CLGGLLTMV (SEQ ID NO 282) (CLG) epitope, corresponding to amino acid 426-434²⁸, and the Lmp1-derived YLQQNWWTL (SEQ ID NO 283) (YLQ) epitope corresponding to amino acid 159-167²⁹, were obtained by stimulation of monocyte-depleted PBL's from the HLA-A2-positive EBV-seropositive donor RG with peptide-pulsed T2 cells, as previously described³². The first stimulation was performed in RPMI 1640 medium containing 10% FCS. A second and a third stimulation were performed in the same conditions on day 7 and day 14. Starting from day 8 the medium was supplemented with 10 U/ml rIL-2 (Chiron, Milan, Italy).

### Cytotoxicity Assay

Target cells were labelled with Na₂⁵¹CrO₄ for 90 min at 37° C. Cytotoxicity tests were routinely run at different effector: target ratios in triplicate. Percent specific lysis was calculated as 100×(cpm sample−cpm medium)/(cpm Triton X-100−cpm medium)²⁷. Spontaneous release was always less than 20%.

### ELISPOT Assay

CTLs (4×10⁴ cells) were plated in triplicate on microplate 96-wells unifilter (Whatman) previously coated with 100 μl of an anti IFN-γ mAb (Endogen, Woburn, Mass.) overnight at 4° C. CTLs were incubated with medium alone as a negative control, with phytohaemagglutinin (PHA) as a positive control, or with 20 μl of each HPLC fractions derived from the in vitro digestion by proteasomes of epitope precursors.

Plates were incubated for 24 h at 37° C., 5% CO₂ and then washed three times with PBS and three times with washing buffer (PBS 0.05% Tween 20) before 100 μl of biotinylated anti-IFN-γ MAb (1 μg/ml; Endogen, Woburn, Mass.) were added, and incubated at 37° C. for 60 min. After the plates were washed again, HRP-conjugated streptavidin (Endogen, Woburn, Mass.) was added and the plates were incubated at room temperature for 45 min. Wells were washed, and individual IFN-γ producing cells were detected using ABC chromogen kit (Sigma, Saint Louise, Mo.). IFN-γ-secreting T cells were counted by direct visualisation. The number of specific IFN-γ-secreting T cells, expressed as spot-forming cells (SFC) per 10⁶ cells, was calculated by subtracting the negative control value. Negative control values were always <500 SFC per 10⁶ input cells.

Animal use was according to national and institutional guidelines. Seven-to-eight week old female Balb/c mice (Nossan, Milan, Italy) were injected with native monomeric biologically active Tat protein (1 μg) resuspended in degassed sterile PBS. Control mice were injected with oxidised Tat (1 μg) or with PBS alone. Samples (100 μl) were given by intramuscular (i.m.) injections in the quadriceps muscles of the posterior legs. Each experimental group consisted of three mice, and the experiment was repeated twice. Mice were boosted at days 11 and 20 after the first injection. Seven days after the last injection, animals were anaesthetised intraperitoneally (i.p.) with 100 μl of isotonic solution containing 1 mg of Inoketan (Virbac, Milan, Italy), and 200 μg Rompun (Bayer, Milan, Italy) and sacrificed to collect spleens. Mononuclear cells from individual spleens were purified using cells strainers, resuspended in PBS containing 20 mM EDTA, and treated with a red blood cells lysis buffer for 4 minutes at room temperature. Cells were washed twice in PBS, lysed and used for western blot analysis as described above.

Seven- to eight-week old female C57BL/6 mice (H-2^{b}) (Nossan) were injected with 25 μg ovalbumin (Sigma, St. Louis, Mo.) alone or in combination with native monomeric biologically active Tat protein (5 and 10 μg) and resuspended in degassed sterile PBS in Freund's adjuvant (CFA for the first injection, and IFA for subsequent injections). Control mice were injected with PBS alone in Freund's adjuvant. Samples (100 μl) were given by subcutaneous (s.c.) injection in one site in the back. Each experimental group consisted of five mice, and the experiment was repeated twice. Mice were boosted at day 24. Two weeks after the last injection, animals were anaesthetised i.p., as described above, and sacrificed to collect spleens. Mononuclear cells from individual spleens were purified as described above, pooled and tested in cytotoxic assay against peptide-pulsed EL4 cells.

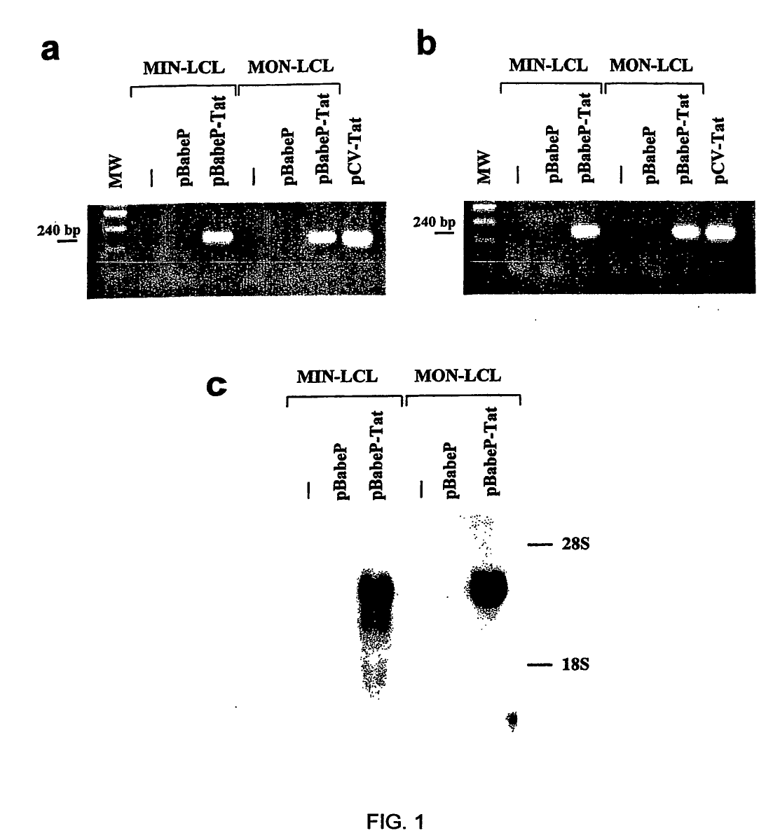

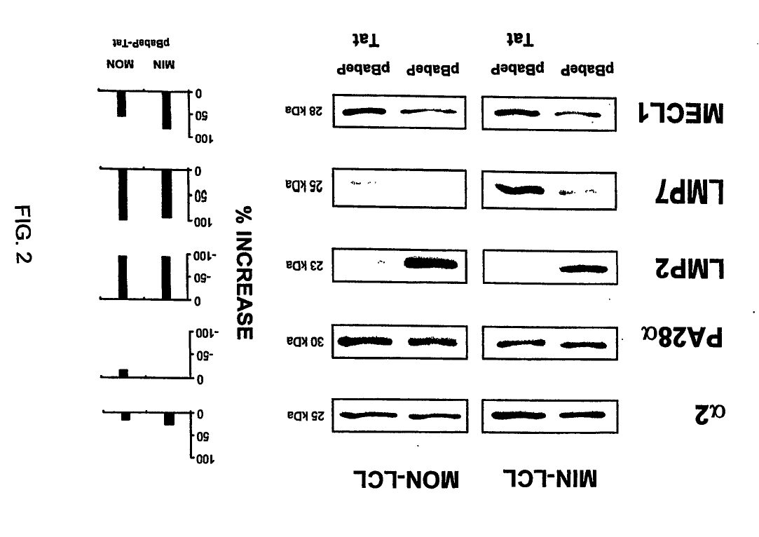

Endogenously expressed Tat modulates proteasome composition and activity To evaluate proteasome expression in the presence of endogenous Tat, lymphoblastoid cell lines (LCL) expressing Tat (MIN-Tat and MON-Tat) were prepared by retroviral transduction and assayed (FIG. 1) for the presence of integrated plasmids and for the expression of Tat RNA as compared to vector transduced cells (MIN-0 and MON-0).

The level of expression of proteasomes was then analysed by Western blot analysis in both Tat-expressing cells as compared to control cells. No difference in proteasome expression was detected in these cells by the use of a monoclonal antibody specific for the α2-subunit (FIG. 2). Since LCL's constitutively express immunoproteasomes¹¹, we then evaluated the expression of the IFNγ-inducible PA28α regulator and of the catalytic β subunits LMP2, LMP7 and MECL1. Both Tat-Tat showed no differences in the expression of PA28α regulator as compared to control cells. In contrast, a marked down-regulation of LMP2 and up-regulation of LMP7 and MECL1 subunits were observed in both Tat-expressing cell lines as compared to the control cells (FIG. 2). A similar increase of LMP7 and MECL1, and decrease of LMP2 were detected in a Jurkat T cell line stably transfected with Tat²² as compared with Jurkat cells transfected with the empty control vector (FIG. 10, Example 2). These findings demonstrate that the subunit composition of immunoproteasomes is affected by the endogenously expressed HIV-1 Tat protein.

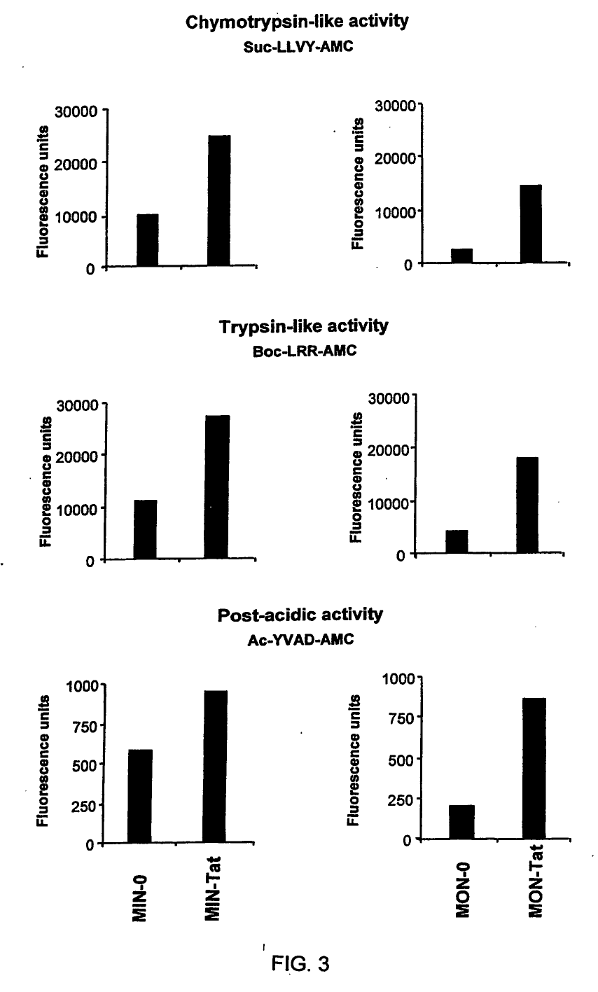

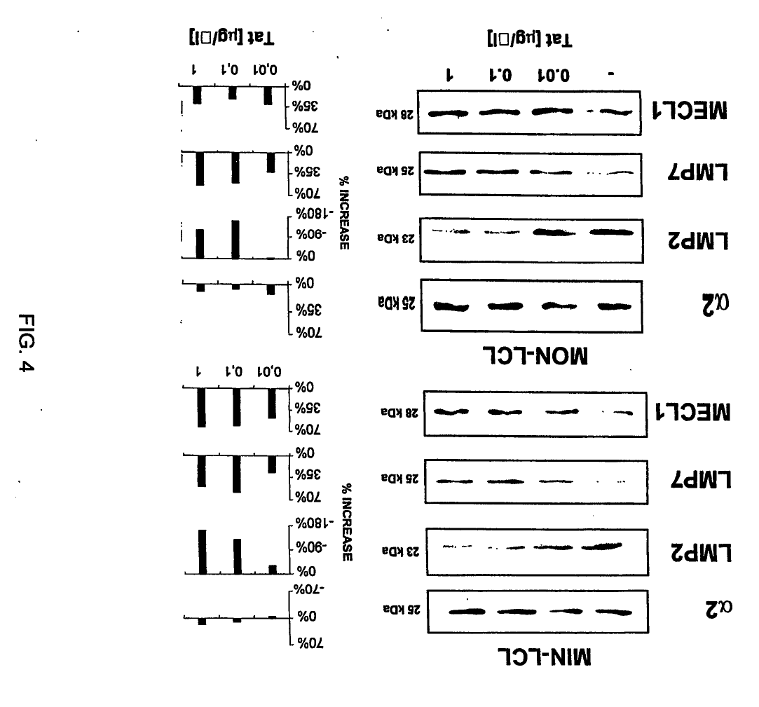

To investigate whether the differences in subunit composition detected by Western blot analysis correlated with differences in enzymatic activity, we analysed the cleavage specificity of equal amount of proteasomes isolated from MIN-Tat and MON-Tat cells or control cells. We tested chymotryptic-like, tryptic-like, and post-acidic activities using Suc-LLVY-AMC, Boc-LRR-AMC and Ac-YVAD-AMC as substrates, respectively. All three enzymatic activities were higher using proteasomes purified from cells expressing Tat as compared to activities of proteasomes purified from control cells (FIG. 3). This observation is in agreement with the results of expression of the three catalytic subunits, since it has been demonstrated that expression of LMP7 and MECL1 is associated with increased chymotryptic and tryptic activities, whereas LMP2 expression is associated with a decreased post-acidic activity^{10,23-25}. Indeed, LCL's expressing Tat showed an increased expression of LMP7 and MECL1 and a decreased expression of LMP2 when compared to control cells.

### Exogenous Biologically Active Tat Modulates Proteasome Composition and Activity

To test the effect on proteasomes in cells after the up-take of exogenous Tat protein, MIN and MON LCL's were cultured in the absence or presence of increasing concentrations of biologically active Tat protein for 24 hours at 37° C. After treatment, the expression of the different subunits was analysed and compared to that of untreated cells. No difference in the expression of the α2-subunit was detected by Western blot analysis, suggesting that exogenous Tat does not alter the expression of proteasomes (FIG. 4) as already observed in cell expressing endogenous Tat (FIG. 2). However, treatment with 0.1-1 μg/ml of Tat determined a down-regulation of LMP2 and an up-regulation of LMP7 and MECL1 as compared to untreated cells (FIG. 4). In addition, proteasomes isolated from MIN and MON LCL's treated with 0.1 μg/ml of Tat presented an increase of all three proteolytic activities as detected using specific fluorogenic peptides (data not shown). These results demonstrate that exogenous Tat protein alters the subunit composition and the proteolytic activity of immunoproteasomes in the cells, as demonstrated for cells endogenously expressing Tat.

### In Vivo Modulation of Proteasome Composition by a Biologically Active Tat

To evaluate the in vivo effect of Tat, we treated Balb/c mice with biologically active Tat or oxidised Tat. After 3 consecutive treatments, splenocytes were isolated and total cell lysates tested for the expression of IFN-γ inducible catalytic subunits.

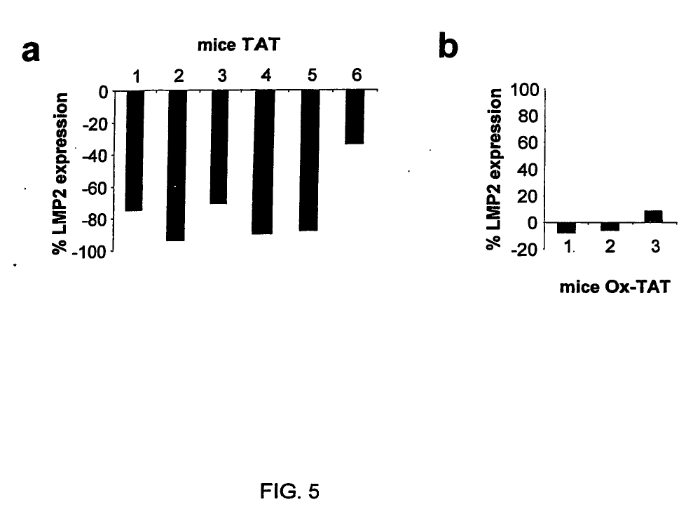

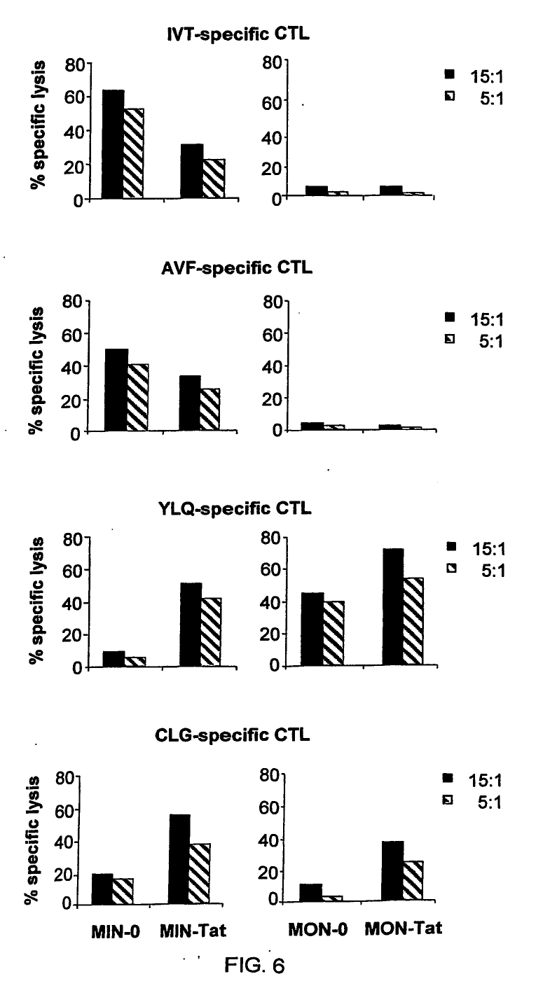

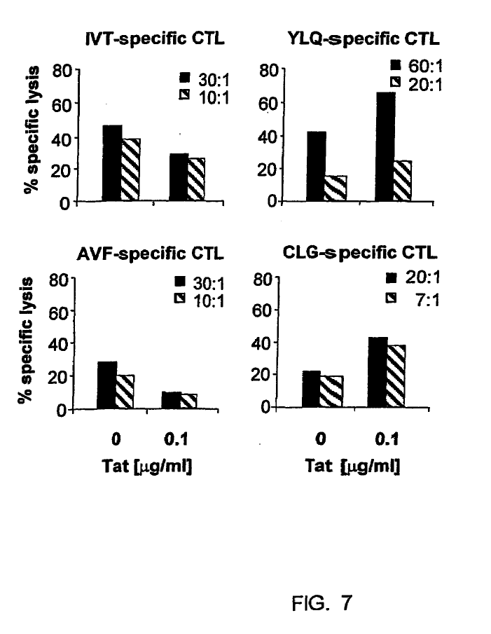

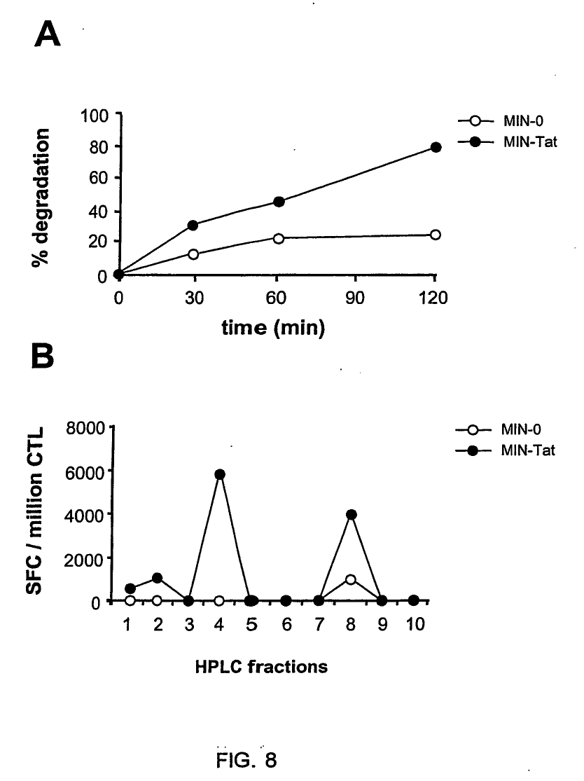

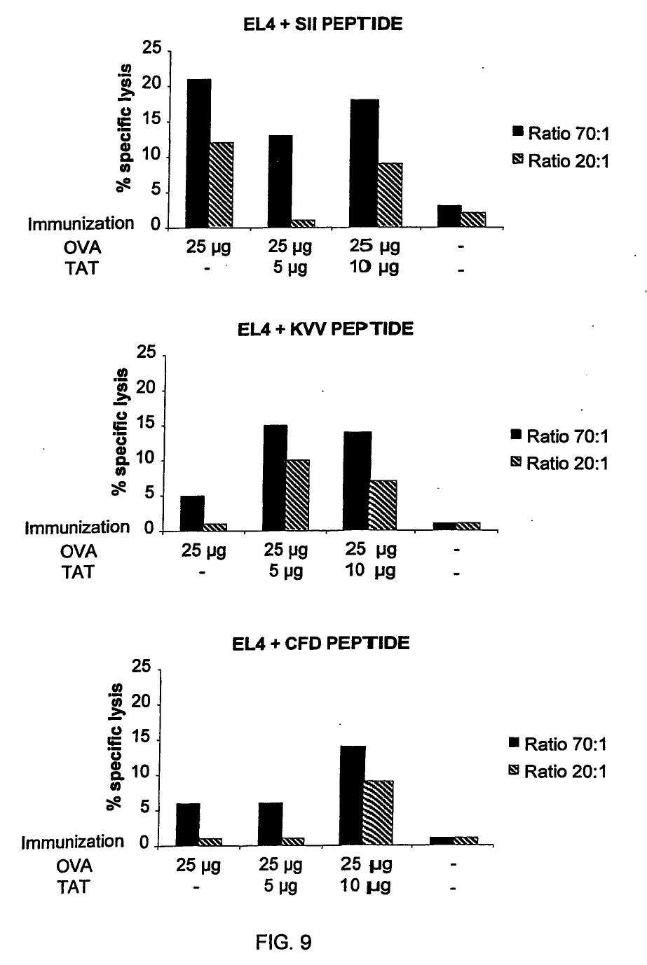

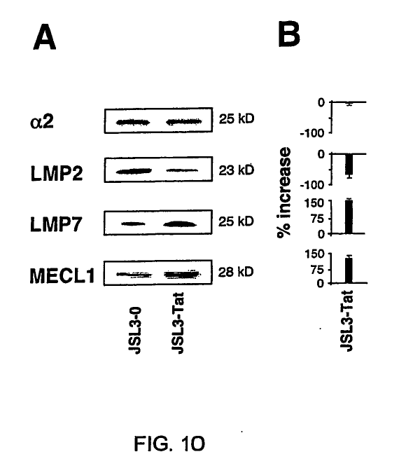

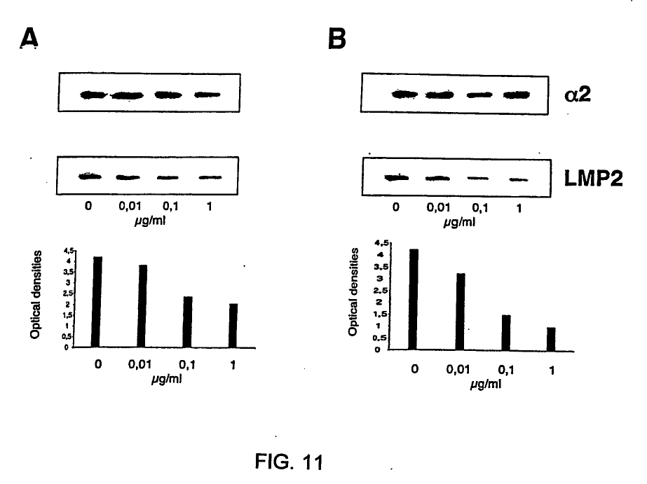

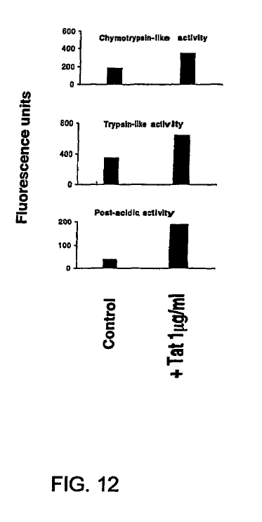

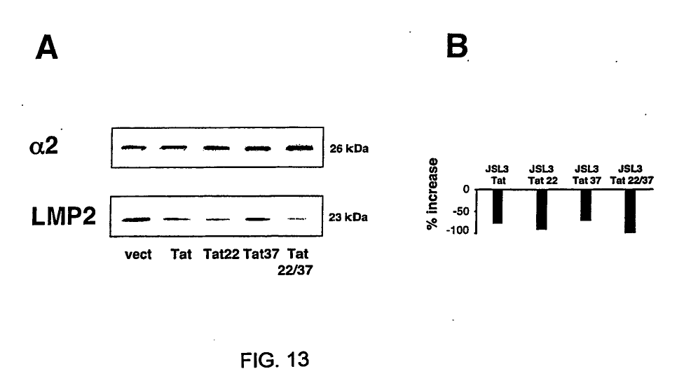

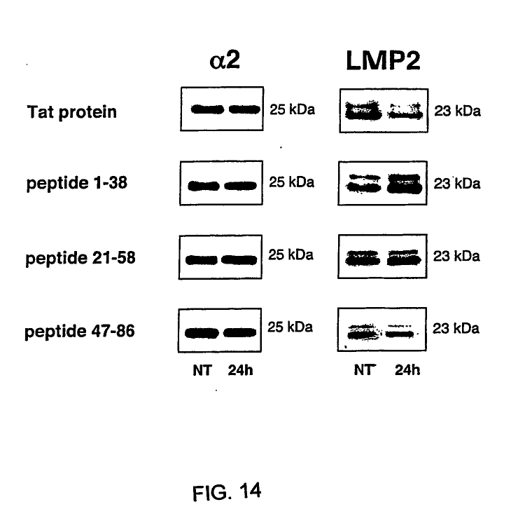

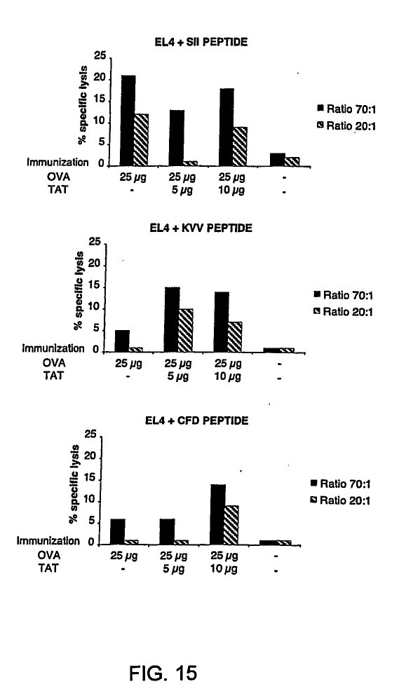

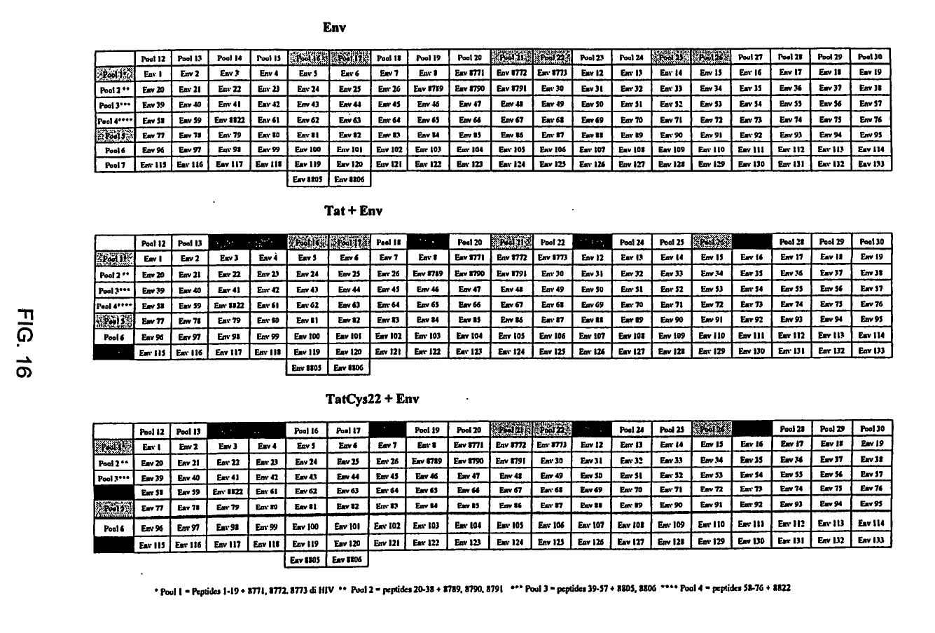

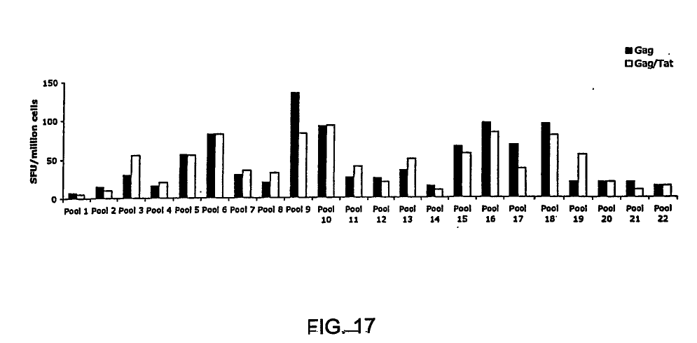

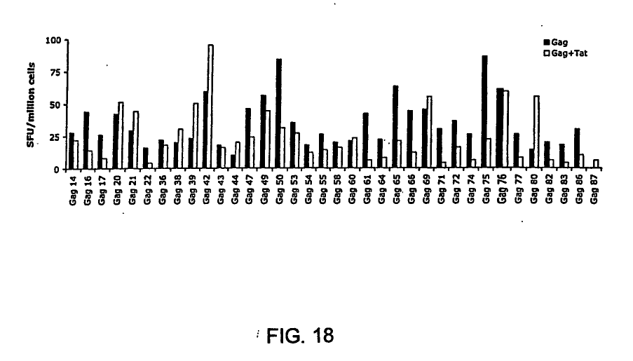

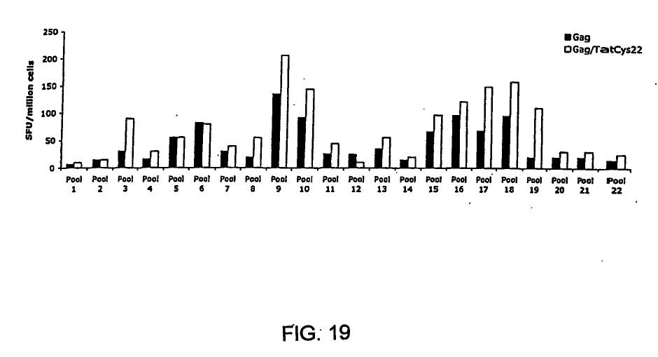

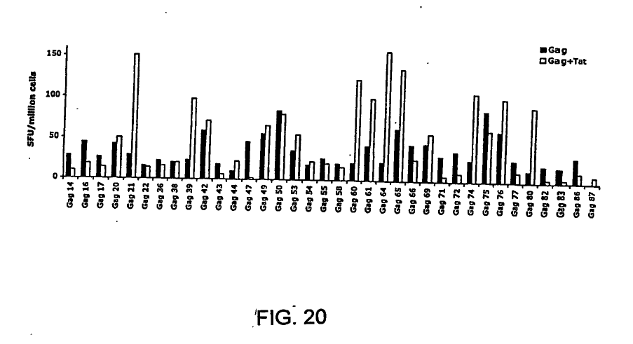

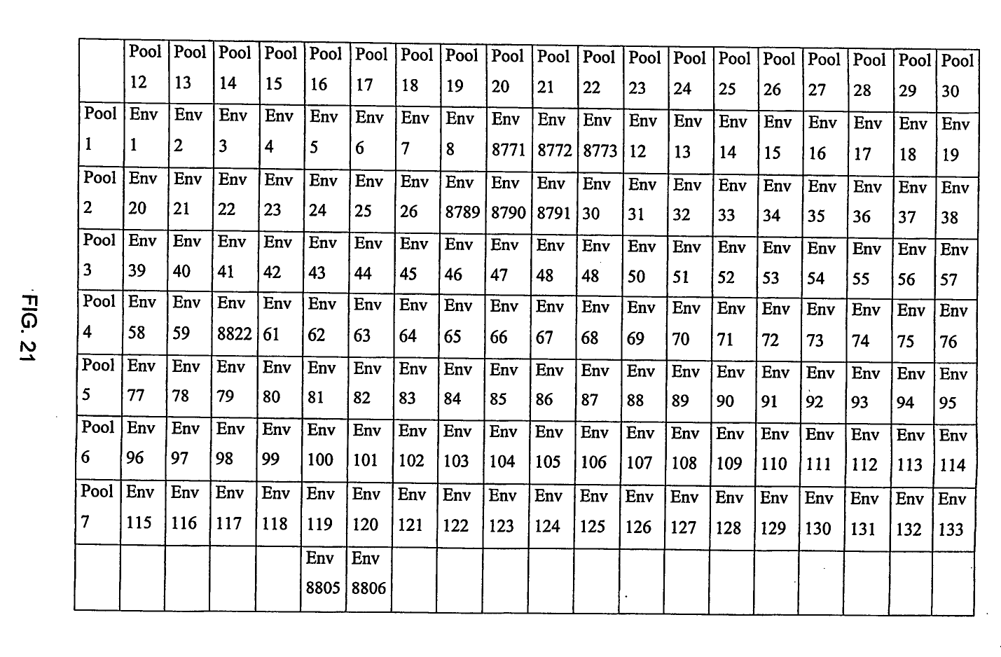

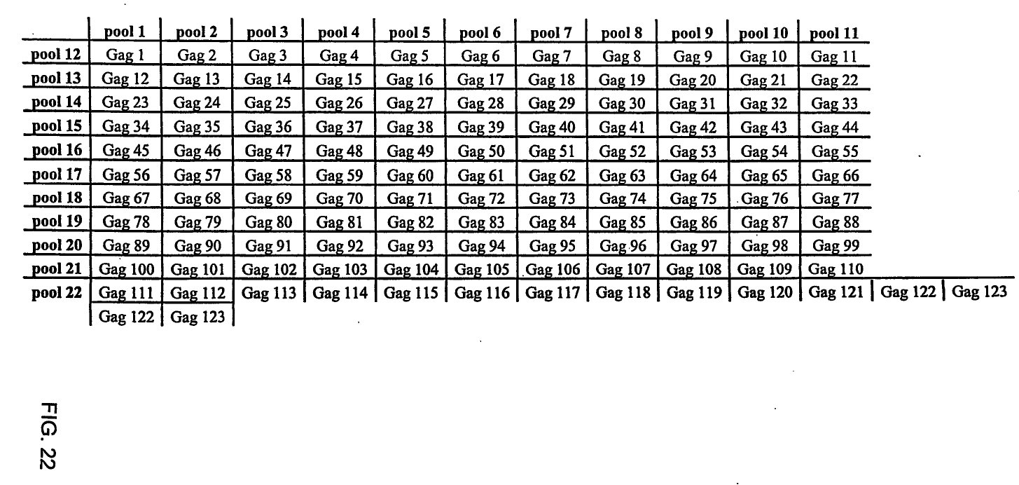

Splenocytes isolated from all mice treated with native Tat demonstrated the down-regulation of LMP2, while no effect was observed in splenocytes isolated from mice treated with oxidised Tat (FIG. 5). These results demonstrate that Tat regulates in vivo the subunit composition of proteasomes.

Tat Modifies the Generation of CTL Peptide Epitopes Derived from EBV Latent Antigens

Since proteasomes play a key role in the generation of CTL epitopes, we investigated the effect of proteasome variation induced by Tat on the generation and presentation of CTL epitopes in LCL's expressing endogenous Tat or exposed to Tat protein.

LCL's express the total set of EBV latent antigens, including nuclear antigens (EBNA) 1, 2, 3, 4, 5, 6 and latent membrane protein (LMP) 1 and 2. These antigens, except for nuclear antigen 1, are targets of cytotoxic T lymphocytes and a large number of CTL epitopes have been identified²⁶. In this set of experiments we evaluated: the immunodominant IVT and AVF HLA-A11-presented epitopes, that derive from the EBNA4 antigen²⁷, and of the subdominant YLQ and CLG epitopes, two HLA-A2-presented epitopes derived from the latent membrane protein 1 (Lmp1) and 2 (Lmp2), respectively^{28,29}. It has been recently shown that the generation of these epitopes depends on proteasome activity^{30,31}. To this purpose, the HLA-2 and -A11 positive MIN LCL and the HLA-A2 positive MON LCL, transduced or not with Tat, were tested as target in cytotoxic assays using CTL cultures specific for the IVT, AVF, CLG and YLQ epitopes (FIG. 6). As demonstrated previously²⁷, IVT- and AVF-specific CTLs efficiently lysed A11-matched LCL, whereas lower levels of specific killing were obtained with YLQ- and CLG-specific CTLs^{28,29,32}. This is due to the poor expression of these two HLA-A2-presented epitopes at the cell surface of EBV-infected B cells²⁶.

The expression of endogenous Tat caused a decrease of IVT- and AVF-specific CTL killing and an increase of YLQ- and CLG-specific killing as compared to control cells transduced with the empty vector (FIG. 6). The HLA-A11-negative MON LCL, either expressing Tat or the empty vector, were not recognised by IVT- and AVF-specific CTL cultures.

In a second set of experiments we evaluated CTL sensitivity of MIN LCL untreated or treated with 0.1 μg/ml of the Tat protein for 24 hours. In agreement with the results of the previous experiments, LCL's treated with Tat were less sensitive to IVT- and AVF-specific CTL killing but were lysed at higher efficiency by YLQ- and CLG-specific CTLs (FIG. 7).

These findings suggest that the effect of Tat on proteasome composition and activity results in changes of epitope presentation at the surface of virally infected cells.

Efficient In Vitro Generation of the CLG Epitope by Proteasomes Purified from Tat-Expressing Cells

To test whether proteasomes from Tat-expressing cells generate the CLG epitope with more efficiency, we analysed the in vitro degradation of a CLG peptide precursor that contains 5 amino acids at the C-terminus (CLG+5) corresponding to the wild-type sequence of the LMP2 antigen. The in vitro assay was performed using proteasomes purified from MIN-Tat and proteasomes purified from MIN-0. The precursor degradation was followed at different time points and evaluated by HPLC analysis. We found that proteasomes isolated from Tat-expressing cells degraded the CLG+5 peptide precursor more efficiently than proteasomes purified from cells expressing the empty vector (FIG. 8a).

To characterise the digestion products, we separated the digests obtained at different time points by HPLC. All fractions were collected and used in ELISPOT to activate CLG-specific CTLs. Fractions purified from digests obtained after 30, 60, and 90 min of incubation did not activate CTL responses (not shown). Only HPLC fractions 4 and 8 obtained after 120 min of degradation with proteasomes purified from MIN-Tat did stimulate CLG-specific CTL responses. This demonstrates that these fractions contain the CLG epitope or a longer but immunogenic epitope. A weak CLG-specific CTL response was also observed in HPLC fraction 8 obtained after 120 min of degradation using proteasomes isolated from MIN-0. These results demonstrate again that proteasomes purified from Tat-expressing cells exhibit a different proteolytic activity and that generate with more efficiency the immunogenic CLG peptide.

### In Vivo Modulation of CTL Responses by Tat

As shown above, Tat alters, both in vitro and in vivo, the subunit composition of proteasomes which, in turn, modulates the presentation of EBV-derived CTL epitopes at the cell surface of EBV-infected B cells. Down-modulation of two immunodominant CTL epitopes and up-regulation of two subdominant CTL epitopes (FIGS. 5 and 6) was observed, suggesting that Tat, by altering the antigen processing machinery, may influence the epitope presentation on the antigen presenting cells affecting immunodominance and subdominance of CTL responses. Accordingly, we decided to evaluate the in vivo effect of Tat on the induction of epitope-specific CTL responses. We used as a model CTL responses directed to ovalbumin (Ova) on the K^{b} background. K^{b}-restricted CTL responses are directed to the immunodominant SIINFEKL (SII) (SEQ ID NO 268) epitope and to the subdominant KVVRFDKL (KVV) (SEQ ID NO 269) and cryptic CFDVFKEL (CFD) (SEQ ID NO 270) epitopes^{33,34}. It has been shown that CTLs specific for KVV are not found upon immunisation of C57BL/6 mice with Ova and that the subdominance of the KVV and CFD epitopes was due to the presence of amino acidic sequences that flank the epitope and that affect the proteasome-mediated processing and the generation of KVV and CFD CTL epitopes^{20,21}. To address whether Tat affects the in vivo generation of the K^{b}-restricted Ova-derived epitopes we vaccinated mice with Ova alone or in combination with Tat.

The presence of specific CTL responses directed to the three Ova-derived epitopes was evaluated on fresh splenocytes using EL4 target cells pulsed or not with the relevant CTL epitopes (FIG. 9). Splenocytes isolated from mice immunised with Ova alone recognised target cells pulsed with the SII epitope but did not recognise cells pulsed with the KVV or CFD epitopes, thereby confirming that CTL responses are mainly directed against the immunodominant SII peptide epitope. In contrast, splenocytes isolated from mice vaccinated with combination Ova/Tat recognised the immunodominant SII epitope less efficiently, but clearly recognised target cells presenting the subdominant KVV and the cryptic CFD epitopes. Control mice did not recognise any peptide-pulsed EL4 cells.

### REFERENCES FOR EXPERIMENT 1 AND THE DESCRIPTION

1. Frankel, A. D. & Pabo, C. O. Cellular uptake of the Tat protein from human immunodeficiency virus. Cell 55, 1189-1193 (1988).
2. Ensoli, B. et al. Release, uptake, and effect of extracellular human immunodeficiency virus type 1 Tat protein on cell growth and viral transactivation. J. Virol. 67, 277-287 (1993).
3. Fanales-Belasio, E. et al. Native HIV-1 Tat protein is selectively taken up by monocyte-derived dendritic cells and induces their maturation, Th-1 cytokine production and antigen presenting function. J. Immunol. 168, 197-206 (2002).
4. Cafaro, A. et al. Control of SHIV-89.6P-infection of cynomolgus monkeys by HIV-1 Tat protein vaccine. Nat. Med. 5, 643-650 (1999).
5. Cafaro, A. et al. Vaccination with DNA containing tat coding sequences and unmethylated CpG motifs protects cynomolgus monkeys upon infection with simian/human immunodeficiency virus (SHIV89.6P). Vaccine 19, 2862-2877 (2001).
6. Pamer, E. & Cresswell, P. Mechanisms of MHC class I-restricted antigen processing. Annu. Rev. Immunol. 16, 323-358 (1998).
7. Rock, K. L. et al. Inhibitors of the proteasome block the degradation of most cell proteins and the generation of peptides presented on MHC class I molecules. Cell 78, 761-771 (1994).
8. Rock, K. L. & Goldberg, A. L. Degradation of cell proteins and the generation of MHC class I-presented peptides. Annu. Rev. Immunol. 17, 739-779 (1999).
9. Voges, D., Zwickl, P. & Baumeister, W. The 26S proteasome: a molecular machine designed for controlled proteolysis. Annu. Rev. Biochem. 68, 1015-1068 (1999).
10. Dick, T. P. et al. Contribution of proteasomal β-subunits to the cleavage of peptide substrates analysed with yeast mutants. J. Biol. Chem. 273, 25637-25646 (1998).
11. Frisan, T., Levitsky, V., Polack, A. & Masucci, M. Phenotype-dependent differences in proteasome subunit composition and cleavage specificity in B cell lines. J. Immunol. 160, 3281-3289 (1998).
12. Morel, S. et al. Processing of some antigens by the standard proteasome but not immunoproteasomes results in poor presentation by dendritic cells. Immunity 12, 107-117 (2000).
13. Sijts, A. J. A. M. et al. Efficient generation of a hepatitis B virus cytotoxic T lymphocyte epitope requires the structural features of immunoproteasomes. J. Exp. Med. 191, 503-513 (2000).
14. Gaczynska, M., Rock, K. L. & Goldberg, A. L. γ-Interferon and expression of MHC genes regulate peptide hydrolysis by proteasomes. Nature (Lond.) 365, 264-267 (1993).
15. Cascio, P., Hilton, C., Kisselev, A. F., Rock, K. L. & Goldberg, A. L. 26S proteasomes and immunoproteasomes produce mainly N-extended versions of an antigenic peptide. EMBO J. 20, 2357-2366 (2001).
16. Serwold, T., F, G., Kim, J., Jacob, R. & Shastri, N. ERAAP customizes peptides for MHC class I molecules in the endoplasmic reticulum. Nature (Lond.) 419, 443-445 (2002).
17. Saric, T. et al. An IFN-γ-induced aminopeptidase in the ER, ERAPI, trims precursors to MHC class I-presented peptides. Nature Immunol. 3, 1169-1176 (2002).
18. York, I. A. et al. The ER aminopeptidase ERAPI enhances or limits antigen-presentation by trimming epitopes to 8-9 residues. Nature Immunol. 3, 1177-1184 (2002).
19. Tanaka, K. & Kasahara, M. The MHC class 1 ligand-generating system: roles of immunoproteasomes and the interferon-γ-inducible proteasome activator PA28. Immunol Rev. 163, 161-176 (1998).
20. Niedermann, G. et al. Contribution of mediated-mediated proteolysis to the hierarchy of epitopes presented by major histocompatibility complex class I molecules. Immunity 2, 289-299 (1995).
21. Mo, A. X. Y., van Lelyveld, S. F. L., Craiu, A. & Rock, K. L. Sequences that flank subdominant and cryptic epitopes influence the proteolytic generation of MHC class I-presented peptides. J. Immunol. 164, 4003-4010 (2000).
22. Caputo, A., Sodroski, J. G. & Haseltine, W. A. Constitutive expression of HIV-1 tat protein in human Jurkat T cells using a BK virus vector. Journal of Acquired Immune Deficiency Syndrome 3, 372-379 (1990).
23. Gaczynska, K., Rock, K. L., Spies, T. & Goldberg, A. L. Peptidase activities of proteasomes are differentially regulated by the major histocompatibility complex-encoded genes for LMP2 and LMP7. Proc. Natl. Acad. Sci. USA 91, 9213-9217 (1994).
24. Groettrup, M. et al. The interferon-γ-inducible 11 S regulator (PA28) and the LMP2/LMP7 subunits govern the peptide production by the 20 S proteasome in vitro. J. Biol. Chem. 270, 23808-23815 (1995).
25. Gaczynska, K., Goldberg, A. L., Tanaka, K., Hendil, K. B. & Rock, K. L. Proteasome subunits X and Y alter peptidase activities in opposite ways to the interferon-γ-induced subunits LMP2 and LMP7. J. Biol. Chem. 271, 17275-17280 (1996).
26. Rickinson, A. B. & Moss, D. J. Human cytotoxic T lymphocyte responses to Epstein-Barr virus infection. Annu. Rev. Immunol. 15, 405-431 (1997).
27. Gavioli, R. et al. Multiple HLA A11-restricted cytotoxic T-lymphocyte epitopes of different immunogenicities in the Epstein-Barr virus-encoded nuclear antigen 4. J. Virol. 67, 1572-1578 (1993).
28. Lee, S. P. et al. HLA A2.1-restricted cytotoxic T cells recognizing a range of Epstein-Barr virus isolates through a defined epitope in latent membrane protein LMP2. J. Virol. 67, 7428-7435 (1993).
29. Khanna, R., Burrows, S. R., Nicholls, J. & Poulsen, L. M. Identification of cytotoxic T cell epitopes within Epstein-Barr virus (EBV) oncogene latent membrane protein 1 (LMP1): evidence for HLA A2 supertype-restricted immune recognition of EBV-infected cells by LMP1-specific cytotoxic T lymphocytes. Eur. J. Immunol. 28, 451-458 (1998).
30. Lautscham, G. et al. Processing of a multiple membrane spanning Epstein-Barr virus protein for CD8+ T cell recognition reveals a dependent-dependent, transporter associated with antigen processing-independent pathway. J. Exp. Med. 194, 1053-1068 (2001).
31. Gavioli, R., Vertuani, S. & Masucci, M. G. Proteasome inhibitors reconstitute the presentation of cytotoxic T cell epitopes in Epstein-Barr virus associated tumors. Int. J. Cancer 101, 532-538 (2002).
32. Micheletti, F. et al. Selective amino acid substitutions of a subdominant Epstein-Barr virus LMP2-derived epitope increase HLA/peptide complex stability and immunogenicity: implications for immunotherapy of Epstein-Barr virus-associated malignancies. Eur. J. Immunol. 29, 2579-2589 (1999).
33. Chen, W., Khilko, S., Fecondo, J., Margulies, D. H. & McCluskey, J. Determinant selection of major histocompatibility complex class I-restricted antigenic peptides is explained by class I-peptide affinity and is strongly influenced by nondominant anchor residues. J. Exp. Med. 180, 1471-1483 (1994).
34. Lipford, G. B., Hoffman, M., Wagner, H. & Heeg, K. Primary in vivo responses to ovalbumin. J. Immunol 150, 1212-1222 (1993).
35. Zeidler, R. et al. Downregulation of TAP1 in B lymphocytes by cellular and Epstein-Barr virus-encoded interleukin-10. Blood 90, 2390-2397 (1997).
36. Chatterjee-Kishore, M., van den Akker, F. & Stark, G. R. Adenovirus E1A down-regulates LMP2 transcription by interfering with the binding of Stat1 to IRF1. J. Biol. Chem. 275, 20406-20411 (2000).
37. Turnell, A. S. et al. Regulation of the 26S proteasome by adenovirus E1A. EMBO J. 19, 4759-4773 (2000).
38. André, P. et al. An inhibitor of HIV-1 protease modulates proteasome activity, antigen presentation, and T cell responses. Proc. Natl. Acad. Sci. USA 95, 13120-13124 (1998).
39. Berezutskaya, B. & Bagchi, S. The human papillomavirus E7 oncoprotein functionally interacts with the S4 subunit of the 26S proteasome. J. Biol. Chem. 272, 30135-30140 (1997).
40. Ehrlich, R. Modulation of antigen processing and presentation by persistent virus infections and in tumors. Hum. Immunol. 54, 104-116 (1997).
41. Schmidtke, G. et al. How an inhibitor of the HIV-I protease modulates proteasome activity. J. Biol. Chem. 274, 35734-35740 (1999).
42. Hu, Z. et al. Hepatitis B virus X protein is both substrate and a potential inhibitor of the proteasome complex. J. Virol. 73, 7231-7240 (1999).
43. Wing, S. S. & Goldberg, A. L. Glucocorticoids activate the ATP-ubiquitin-dependent proteolytic system in skeletal muscle during fasting. American Journal of Physiology 264, 668-676 (1993).
44. Gavioli, R., Frisan, T., Vertuani, S., Bornkamm, G. W. & Masucci, M. G. c-Myc overexpression activates alternative pathways for intracellular proteolysis in lymphoma cells. Nat. Cell Biol. 3, 283-288 (2001).
45. Cerundolo, V., Kelly, A., Elliot, T., Trowsdale, J. & Townsend, A. Genes encoded in the major histocompatibility complex affecting the generation of peptides for TAP transport. Eur. J. Immunol. 25, 554-562 (1995).
46. Sewell, A. K. et al. IFN-γ exposes a cryptic cytotoxic T lymphocyte epitope in HIV-1 reverse transcriptase. J. Immunol. 162, 7075-7079 (1999).
47. van Hall, T. et al. Differential influence on cytotoxic T lymphocyte epitope presentation by controlled expression of either proteasome immunosubunits or PA28. J. Exp. Med. 192, 483-494 (2000).
48. Sijts, A. J. A. M. et al. MHC class I antigen processing of an adenovirus CTL epitope is linked to the levels of immunoproteasomes in infected cells. J. Immunol. 164, 4500-4506 (2000).
49. Schmidtke, G. et al. Inactivation of a defined active site in the mouse 20S proteasome complex enhances major histocompatibility complex class I antigen presentation of a murine cytomegalovirus protein. J. Exp. Med. 187, 1641-1646 (1998).
50. Chen, W., Norbury, C. C., Cho, Y., Yewdell, J. W. & Bennink, J. R. Immunoproteasomes shape immunodominance hierarchies of antiviral CD8+ T cells at the levels of T cell repertoire and presentation of viral antigens. J. Exp. Med. 193, 1319-1326 (2001).
51. Groll, M. et al. The catalytic sites of 20S proteasomes and their role in subunit maturation: a mutational and crystallographic study. Proc. Natl. Acad. Sci. USA 96, 10976-10983 (1999).
52. Feltkamp, M. C. W. et al. Cytotoxic T lymphocytes raised against a subdominant epitope offered as a synthetic peptide eradicate human papilloma virus type 16-induced tumors. Eur. J. Immunol. 25, 2638-2642 (1995).
53. Rubartelli, A., Poggi, A., Sitia, R. & Zocchi; M. R. HIV-I Tat: a polypeptide for all seasons. Immunol. Today 19, 543-545 (1998).
54. Miller, A. D. et al. Construction and properties of retrovirus packaging cells based on gibbon ape leukemia virus. J. Virol. 65, 2220-2224 (1991).
55. Morgenstern, J. P. & Land, H. Advanced mammalian gene transfer: high titre retroviral vectors with multiple drug selection markers and a complementary helper-free packaging cell line. Nucleic Acids Res. 18, 3587-3596 (1990).
56. Graham, F. L. & van der Eb, A. J. A new technique for the assay of infectivity of human adenovirus 5 DNA. Virology 52, 456-467 (1973).
57. Micheletti, F. et al. Supra-agonist peptides enhance the reactivation of memory cytotoxic T lymphocyte responses. J. Immunol. 165, 4264-4271 (2000).

### Example 2

### HIV-1 Tat-Mutant and an HIV Tat-Derived Peptides Modulate Proteasome Composition and Enzymatic Activity

### Materials and Methods

### Cells

Jurkat T cells expressing Tat or mutated Tat (referred to as Tat22, wherein a Cys at position 22 is mutated) have previously been described (1). Cells were cultured in a medium supplemented with 800 μg/ml neomycin (Sigma, St. Louise, Mich.).

### HIV-1 Tat Protein

HIV-1 Tat from the human T lymphotropic virus type IIIB isolate (BH10 clone) was expressed in E. Coli and purified by heparin-affinity chromatography and HPLC as previously described (2). The lyophilised Tat protein was stored at −80° C. to prevent oxidation, reconstituted in degassed buffer before use, and handled as described (3). Different lots of Tat were used with reproducible results, and, in all cases, endotoxin concentration was undetectable (detection threshold: 0.05 EU/μg).

### Purification of Proteasomes

Cells were lysed with glass beads as previously described (4). Supernatants were ultracentrifuged for 1 h at 100,000 g and loaded into an affinity column containing a matrix derivatised with the MCP21 mAb specific for the α2 subunit of the proteasome (Affinity, Exeter, UK). Proteasomes were eluted with 25 mM Tris-HCl pH 7.5 containing 2 M NaCl, and 0.5 ml fractions were collected. Homogeneity of the eluted material was confirmed by analysis of an aliquot by SDS 12% PAGE and Coomassie blue staining of the gel. Fractions containing proteasomes were combined and dialysed against 25 mM Tris-HCl pH 7.5. Protein concentration was determined using the BCA method (Pierce Chemical, Rockford, Ill.).

### Western Blot Assay

Equal amounts of proteins, or equal amounts of purified proteasomes, were loaded on a 12% SDS-PAGE and electro-blotted onto Protran nitrocellulose membranes (Schleicher & Schuell, Keene, Hampshire, USA). Blots were probed with Abs specific for α2, LMP2 LMP7, MECL1, and PA28α subunits (Affinity), and developed by enhanced chemi-luminescence (ECL, Amersham Pharmacia Biotech, Uppsala, SW).

### Enzymatic Assays

The chymotrypsin-like, trypsin-like and post-acidic activities of purified proteasomes were tested using the fluorogenic substrates Suc-LLVY-AMC, Boc-LRR-AMC and Ac-YVAD-AMC, respectively, as previously described (4). Fluorescence was determined by a fluorimeter (Spectrafluor plus, Tecan, Salzburg, Austria). Proteasome activity is expressed as arbitrary fluorescence units.

### Synthetic Peptides

Peptides were synthesized by the solid phase method and purified by HPLC to >98% purity, as previously described (5). Structure verification was performed by elemental and amino acid analysis and mass spectrometry. Peptides were dissolved in DMSO at 10⁻² M, kept at −20° C., and diluted in PBS before use.

### Results and Discussion

### Endogenously Expressed Tat or Exogenous Native Tat Protein Modulate Proteasome Composition and Activity in Jurkat Cells

We have shown in Example 1 that the HIV-1 Tat modifies the catalytic subunit composition and activity of immunoproteasomes in lymphoblastoid cell lines which either express Tat or have been treated with exogenous biological active Tat protein. Similarly, the endogenous expression of Tat in Jurkat cells induces down-regulation of LMP2 and up-regulation of LMP7 and MECL1 (see Example 1 and FIG. 10).

To assay whether the exogenous Tat protein modulates the expression of the catalytic subunits of proteasomes in Jurkat cells, we evaluated the expression of proteasomes from Jurkat cells cultured for 12 (FIG. 11A) and 24 hrs (FIG. 11B) in the absence or presence of increasing concentrations of the native Tat protein. After treatment, expression of LMP2 subunit from purified proteasomes was evaluated as a marker of Tat-induced proteasomal modification. As shown in FIG. 11, maximal down-modulation of the LMP2 subunit was observed after 24 h of treatment with 0.1-1 μg/ml of Tat.

These results demonstrate that both endogenously expressed Tat and exogenous native Tat protein modify the subunit composition of immunoproteasomes in Jurkat cells.

To investigate whether the differences in subunit composition correlated with differences in enzymatic activity, we analysed the cleavage specificity of equal amounts of proteasomes purified from Jurkat cells treated for 24 h with 1 μg/ml Tat protein or from control cells. Chymotryptic-like, tryptic-like, and post-acidic activities were all augmented in proteasomes purified from Jurkat cells treated with Tat, as compared to control cells (FIG. 12).

Tat does not require Cysteine 22 to modulate proteasome composition and activity

In the next set of experiments we evaluated the effect of Tat mutants stably expressed in Jurkat cells. Cysteines at position 22 and/or 37 were substituted with glycine and serine, respectively, to obtain three mutant Tat molecules (Tat22, Tat37 and Tat22/37). Tat22 and Tat22/37 mutants, in contrast to wild-type Tat, have no effect on the transactivation of the HIV-1 LTR, and do not induce reactivation of latent infection.

The level of expression of proteasomes was then analysed in Tat-expressing cells and compared to cells expressing Tat mutants. No difference in proteasome expression was detected in these cells by the use of a monoclonal antibody specific for the α2-subunit (FIG. 13). In contrast, a marked down-regulation of the LMP2 subunit was observed in proteasomes purified from cells expressing Tat mutants, as previously demonstrated for cells expressing wild-type Tat.

The Tat-derived 47-86 peptide is sufficient to down-modulate the LMP2 subunit To identify the region of Tat responsible for the modulation of the catalytic subunits of immuno-proteasomes, we tested the effect of peptides 1-38, 21-58 and 47-86 covering the wild-type sequence of Tat. Jurkat cells were treated for 24 h with 0.1 μg/ml of Tat-derived peptides and, after treatment, total cell lysates were assayed for proteasome expression by western-blot. As shown in FIG. 14, Tat protein and peptides 47-86 induced down-regulation of LMP2, while the other peptides showed no discernable effect.

### Conclusions

We have shown here that a mutated form of the HIV-1 Tat protein, carrying a glycine instead of cysteine 22 (Tat22), like wild-type Tat, modifies the subunit composition of proteasomes. In addition, we demonstrated that peptide 47-86, derived from the wild-type Tat, protein is sufficient to down-modulate LMP2 subunit. We have recently shown that LMP2 down-regulation by wild-type Tat results in a different generation of CTL epitopes in virally infected cells (6). Furthermore, we have produced evidence suggesting that Tat modifies in vivo CTL responses against heterologous antigens favouring the generation of subdominant CTL epitopes (unpublished results). Therefore, mutated forms of Tat or Tat-derived peptide may represent an important alternative to the use of wild-type Tat in vaccination strategies aimed at increasing epitope-specific T cell responses directed to heterologous antigens.

### REFERENCES FOR EXAMPLE 2

1. Caputo, A., J. G. Sodroski, and W. A. Haseltine. 1990. Constitutive expression of HIV-1 tat protein in human Jurkat T cells using a BK virus vector. Journal of Acquired Immune Deficiency Syndrome 3:372.
2. Fanales-Belasio, E., S. Moretti, F. Nappi, G. Barillari, F. Micheletti, A. Cafaro, and B. Ensoli. 2002. Native HIV-1 Tat protein is selectively taken up by monocyte-derived dendritic cells and induces their maturation, Th-1 cytokine production and antigen presenting function. J. Immunol. 168:197.
3. Ensoli, B., L. Buonaguro, G. Barillari, V. Fiorelli, R. Gendelman, R. A. Morgan, P. Wingfield, and R. C. Gallo. 1993. Release, uptake, and effect of extracellular human immunodeficiency virus type 1 Tat protein on cell growth and viral transactivation. J. Virol. 67:277.
4. Gavioli, R., T. Frisan, S. Vertuani, G. W. Bornkamm, and M. G. Masucci. 2001. c-Myc overexpression activates alternative pathways for intracellular proteolysis in lymphoma cells. Nat. Cell Biol. 3:283.
5. Micheletti, F., A. Canella, S. Vertuani, M. Marastoni, L. Tosi, S. Volinia, S. Traniello, and R. Gavioli. 2000. Supra-agonist peptides enhance the reactivation of memory cytotoxic T lymphocyte responses. J. Immunol. 165:4264.
6. Gavioli, R., E. Gallerani, C. Fortini, M. Fabris, A. Bottoni, A. Canella, A. Bonaccorsi, M. Marastoni, F. Micheletti, A. Cafaro, P. Rimessi, A. Caputo, and B. Ensoli. 2004. HIV-1 Tat protein modulates cytotoxic T cell epitopes by modifying proteasome composition and enzymatic activity. J. Immunol. 173:3838.

### Example 3

### HIV-1 Tat Protein Increases Cytotoxic T Cell Epitopes Recognized within Heterologous HIV-1 Structural Gag and Env Antigens

### Introduction

We shown above that the HIV-1 Tat protein modulates in vitro CTL epitope hierarchy by modifying the catalytic subunit composition of immunoproteasome. In particular, by up-regulating LMP7 and MECL1 subunits and by down-modulating the LMP2 subunit, both intracellularly expressed or exogenous native Tat protein increase the major proteolytic activities of the proteasome resulting in a more efficient generation and presentation of subdominant CTL epitopes. Since the amount of MHC-I/epitope complexes is crucial in determining the presence and the strength of epitope-specific CTL responses and to verify the biological relevance of these findings for vaccination strategies, we evaluated epitope-specific CTL responses against ovalbumin in mice vaccinated with both Tat and ovalbumin.

We found that Tat slightly decreases CTL responses directed to the immunodominant epitope while induces those directed to subdominant and cryptic T-cell epitopes that were not present in mice vaccinated with ovalbumin alone. Due to these effects we exploited the effect of Tat on T cell responses against structural HIV gene products. We found that Tat increases the number of CTL epitopes within Gag and Env antigens. Thus, Tat may represent a new tool to induce new epitope-specific CTL responses against HIV antigens and could be used as co-antigen for the development of new vaccination strategies against AIDS.

### Materials and Methods

### HIV-1 Proteins.

HIV-1 Tat and the mutant Tatcys22 (C→G) from the human T lymphotropic virus type IIIB isolate (BH10 clone) was expressed in E. Coli and purified by heparin-affinity chromatography and HPLC as described previously (2). The Tat proteins were stored lyophilised at −80° C. to prevent oxidation, reconstituted in degassed buffer before use, and handled as described (4). Different lots of Tat were used with reproducible results, and in all cases endotoxin concentration was undetectable (detection threshold: 0.05 EU/μg). HIV-1 GagSF2 and HIV-1 EnvSF2 proteins were obtained from Chiron and NIH AIDS Reagent Program respectively (HIV-1 gp120 SF162; #7363). The Gag sequence (HIVSF2 p55) is given in SEQ ID NO 266. The Env sequence (HIV-1 SF162 gp120) is given in SEQ ID NOS 267 (without linker) and 288 (with linker). The entire HIV-1 Env gp160 SF162 sequence is given in SEQ ID NO. 287.

### Synthetic Peptides.

Peptides were synthesized by solid phase method and purified by HPLC to >98% purity, as previously described (5). Structure verification was performed by elemental and amino acid analysis and mass spectrometry.

Gag and Env peptides, 15 amino acid long and overlapping by 10 to 11 amino acids, spanning the entire Gag (HIV-1 consensus subtype B Gag complete set, #8117) and Env sequences (SHIV SF162P3 env set; #7619 and HIV-1 consensus subtype B Env complete set, #9840), were provided by NIH AIDS Reagent Program. Peptides were dissolved in DMSO at 10⁻³ M, kept at −20° C., and diluted in PBS before use. The Gag peptides are listed in Table 1 and the Env peptides are listed in Table 2. The amino acid number relative to the full sequences are given, together with the appropriate SEQ ID NO from the Sequence Listing.

TABLE 1
Gag peptides

| | | | |
|---|---|---|---|
| Reference | | Amino Acid | |
| Name | Sequence | Position | SEQ ID NO. |

| | | | |
|---|---|---|---|
| Gag 1 | MGAPASVLSGGELDR | 1-15 | 1 |
| Gag 2 | ASVLSGGELDRWEKI | 5-19 | 2 |
| Gag 3 | SGGELDRWEKIRLRP | 9-23 | 3 |
| Gag 4 | LDRWEKIRLRPGGKK | 13-27 | 4 |
| Gag 5 | EKIRLRPGGKKKYKL | 17-31 | 5 |
| Gag 6 | LRPGGKKKYKLKHIV | 21-35 | 6 |
| Gag 7 | GKKKYKLKHIVWASR | 25-39 | 7 |
| Gag 8 | YKLKHIVWASRELER | 29-43 | 8 |
| Gag 9 | HIVWASRELERFAVN | 33-47 | 9 |
| Gag 10 | ASRELERFAVNPGLL | 37-51 | 10 |
| Gag 11 | LERFAVNPGLLETSE | 41-55 | 11 |
| Gag 12 | AVNPGLLETSEGCRQ | 45-59 | 12 |
| Gag 13 | GLLETSEGCRQILGQ | 49-63 | 13 |
| Gag 14 | TSEGCRQILGQLQPS | 53-67 | 14 |
| Gag 15 | CRQILGQLQPSLQTG | 57-71 | 15 |
| Gag 16 | LGQLQPSLQTGSEEL | 61-75 | 16 |
| Gag 17 | QPSLQTGSEELRSLY | 65-79 | 17 |
| Gag 18 | QTGSEELRSLYNTVA | 69-83 | 18 |
| Gag 19 | EELRSLYNTVATLYC | 73-87 | 19 |
| Gag 20 | SLYNTVATLYCVHQR | 77-91 | 20 |
| Gag 21 | TVATLYCVHQRIEVK | 81-95 | 21 |
| Gag 22 | LYCVHQRIEVKDTKE | 85-99 | 22 |
| Gag 23 | HQRIEVKDTKEALEK | 89-103 | 23 |
| Gag 24 | EVKDTKEALEKIEEE | 93-107 | 24 |
| Gag 25 | TKEALEKIEEEQNKS | 97-111 | 25 |
| Gag 26 | LEKIEEEQNKSKKKA | 101-115 | 26 |
| Gag 27 | EEEQNKSKKKAQQAA | 105-119 | 27 |
| Gag 28 | NKSKKKAQQAAADTG | 109-123 | 28 |
| Gag 29 | KKAQQAAADTGNSSQ | 113-127 | 29 |
| Gag 30 | QAAADTGNSSQVSQN | 117-131 | 30 |
| Gag 31 | DTGNSSQVSQNYPIV | 121-135 | 31 |
| Gag 32 | SSQVSQNYPIVQNLQ | 125-139 | 32 |
| Gag 33 | SQNYPIVQNLQGQMV | 129-143 | 33 |
| Gag 34 | PIVQNLQGQMVHQAI | 133-147 | 34 |
| Gag 35 | NLQGQMVHQAISPRT | 137-151 | 35 |
| Gag 36 | QMVHQAISPRTLNAW | 141-155 | 36 |
| Gag 37 | QAISPRTLNAWVKVV | 145-159 | 37 |
| Gag 38 | PRTLNAWVKVVEEKA | 149-463 | 38 |
| Gag 39 | NAWVKVVEEKAFSPE | 153-167 | 39 |
| Gag 40 | KVVEEKAFSPEVIPM | 157-171 | 40 |
| Gag 41 | EKAFSPEVIPMFSAL | 161-175 | 41 |
| Gag 42 | SPEVIPMFSALSEGA | 165-179 | 42 |
| Gag 43 | IPMFSALSEGATPQD | 169-183 | 43 |
| Gag 44 | SALSEGATPQDLNTM | 173-187 | 44 |
| Gag 45 | EGATPQDLNTMLNTV | 177-191 | 45 |
| Gag 46 | PQDLNTMLNTVGGHQ | 181-195 | 46 |
| Gag 47 | NTMLNTVGGHQAAMQ | 185-199 | 47 |
| Gag 48 | NTVGGHQAAMQMLKE | 189-203 | 48 |
| Gag 49 | GHQAAMQMLKETINE | 193-207 | 49 |
| Gag 50 | AMQMLKETINEEAAE | 197-211 | 50 |
| Gag 51 | LKETINEEAAEWDRL | 201-215 | 51 |
| Gag 52 | INEEAAEWDRLHPVH | 205-219 | 52 |
| Gag 53 | AAEWDRLHPVHAGPI | 209-223 | 53 |
| Gag 54 | DRLHPVHAGPIAPGQ | 213-227 | 54 |
| Gag 55 | PVHAGPIAPGQMREP | 217-23 1 | 55 |
| Gag 56 | GPIAPGQMREPRGSD | 221-235 | 56 |
| Gag 57 | PGQMREPRGSDIAGT | 225-239 | 57 |
| Gag 58 | REPRGSDIAGTTSTL | 229-243 | 58 |
| Gag 59 | GSDIAGTTSTLQEQI | 233-247 | 59 |
| Gag 60 | AGTTSTLQEQIGWMT | 237-251 | 60 |
| Gag 61 | STLQEQIGWMTNNPP | 241-255 | 61 |
| Gag 62 | EQIGWMTNNPPIPVG | 245-259 | 62 |
| Gag 63 | WMTNNPPIPVGEIYK | 249-263 | 63 |
| Gag 64 | NPPIPVGEIYKRWII | 253-267 | 64 |
| Gag 65 | PVGEIYKRWIILGLN | 257-271 | 65 |
| Gag 66 | IYKRWIILGLNKIVR | 261-275 | 66 |
| Gag 67 | WIILGLNKIVRMYSP | 265-279 | 67 |
| Gag 68 | GLNKIVRMYSPTSIL | 269-283 | 68 |
| Gag 69 | IVRMYSPTSILDIRQ | 273-287 | 69 |
| Gag 70 | YSPTSILDIRQGPKE | 277-291 | 70 |
| Gag 71 | SILDIRQGPKEPFRD | 281-295 | 71 |
| Gag 72 | IRQGPKEPFRDYVDR | 285-299 | 72 |
| Gag 73 | PKEPFRDYVDRFYKT | 289-303 | 73 |
| Gag 74 | FRDYVDRFYKTLRAE | 293-307 | 74 |
| Gag 75 | VDRFYKTLRAEQASQ | 297-311 | 75 |
| Gag 76 | YKTLRAEQASQEVKN | 301-315 | 76 |
| Gag 77 | RAEQASQEVKNWMTE | 305-319 | 77 |
| Gag 78 | ASQEVKNWMTETLLV | 309-323 | 78 |
| Gag 79 | VKNWMTETLLVQNAN | 313-327 | 79 |
| Gag 80 | MTETLLVQNANPDCK | 317-331 | 80 |
| Gag 81 | LLVQNANPDCKTILK | 321-335 | 81 |
| Gag 82 | NANPDCKTILKALGP | 325-339 | 82 |
| Gag 83 | DCKTILKALGPAATL | 329-343 | 83 |
| Gag 84 | ILKALGPAATLEEMM | 333-347 | 84 |
| Gag 85 | LGPAATLEEMMTACQ | 337-351 | 85 |
| Gag 86 | ATLEEMMTACQGVGG | 341-355 | 86 |
| Gag 87 | EMMTACQGVGGPGHK | 345-359 | 87 |
| Gag 88 | ACQGVGGPGHKARVL | 349-363 | 88 |
| Gag 89 | VGGPGHKARVLAEAM | 353-367 | 89 |
| Gag 90 | GHKARVLAEANSQVT | 357-371 | 90 |
| Gag 91 | RVLAEAMSQVTNSAT | 361-375 | 91 |
| Gag 92 | EAMSQVTNSATIMMQ | 365-379 | 92 |
| Gag 93 | QVTNSATIMMQRGNF | 369-383 | 93 |
| Gag 94 | SATIMMQRGNFRNQR | 373-3 87 | 94 |
| Gag 95 | MMQRGNFRNQRKTVK | 377-391 | 95 |
| Gag 96 | GNFRNQRKTVKCFNC | 381-395 | 96 |
| Gag 97 | NQRKTVKCFNCGKEG | 385-399 | 97 |
| Gag 98 | TVKCFNCGKEGHIAK | 389-403 | 98 |
| Gag 99 | FNCGKEGHIAKNCRA | 393-407 | 99 |
| Gag 100 | KEGHIAKNCRAPRKK | 397-411 | 100 |
| Gag 101 | IAKNCRAPRKKGCWK | 401-415 | 101 |
| Gag 102 | CRAPRKKGCWKCGKE | 405-419 | 102 |
| Gag 103 | RKKGCWKCGKEGHQM | 409-423 | 103 |
| Gag 104 | CWKCGKEGHQMKDCT | 413-427 | 104 |
| Gag 105 | GKEGHQMKDCTERQA | 417-43 1 | 105 |
| Gag 106 | HQMKDCTERQANFLG | 421-435 | 106 |
| Gag 107 | DCTERQANFLGKIWP | 425-439 | 107 |
| Gag 108 | RQANFLGKIWPSHKG | 429-443 | 108 |
| Gag 109 | FLGKIWPSHKGRPGN | 433-447 | 109 |
| Gag 110 | IWPSHKGRPGNFLQS | 437-451 | 110 |
| Gag 111 | HKGRPGNFLQSRPEP | 441-455 | 111 |
| Gag 112 | PGNFLQSRPEPTAPP | 445-459 | 112 |
| Gag 113 | LQSRPEPTAPPEESF | 449-463 | 113 |
| Gag 114 | PEPTAPPEESFRFGE | 453-467 | 114 |
| Gag 115 | APPEESFRFGEETTT | 457-471 | 115 |
| Gag 116 | ESFRFGEETTTPSQK | 461-475 | 116 |
| Gag 117 | FGEETTTPSQKQEPI | 465-479 | 117 |
| Gag 118 | TTTPSQKQEPIDKEL | 469-483 | 118 |
| Gag 119 | SQKQEPIDKELYPIA | 473-487 | 119 |
| Gag 120 | EPIDKELYPLASLRS | 477-491 | 120 |
| Gag 121 | KELYPLASLRSLFGN | 481-495 | 121 |
| Gag 122 | PLASLRSLFGNDPSS | 485-499 | 122 |
| Gag 123 | LRSLFGNDPSSQ | 489-500 | 123 |

TABLE 2
Env peptides

| Reference | | Amino Acid | |
|---|---|---|---|
| Name | Sequence | Position | SEQ ID NO. |
| Env 1 | MRVKGIRKNYQHLWR | aa 1-15 | 124 |
| Env 2 | GIRKNYQHLWRGGTL | aa 5-19 | 125 |
| Env 3 | NYQHLWRGGTLLLGM | aa 9-23 | 126 |
| Env 4 | LWRGGTLLLGMLMIC | aa 13-27 | 127 |
| Env 5 | GTLLLGMLMICSAVE | aa 17-31 | 128 |
| Env 6 | LGMLMICSAVEKLWV | aa 21-35 | 129 |
| Env7 | MICSAVEKLWVTVYY | aa 25-39 | 130 |
| Env 8 | AVEKLWVTVYYGVPA | aa 29-43 | 131 |
| Env 9 | LWVTVYYGVPAWKEA | aa 33-47 | 132 |
| Env 10 | VYYGVPAWKEATTTL | aa 37-51 | 133 |
| Env 11 | VPAWKEATTTLFCAS | aa 41-55 | 134 |
| Env 12 | KEATTTLFCASDAKA | aa 45-59 | 135 |
| Env 13 | TTLFCASDAKAYDTE | aa 49-63 | 136 |
| Env 14 | CASDAKAYDTEVHNV | aa 53-67 | 137 |
| Env 15 | AKAYDTEVHNVWATH | aa 57-71 | 138 |
| Env 16 | DTEVHNVWATHACVP | aa 61-75 | 139 |
| Env 17 | HNVWATHACVPTDPN | aa 65-79 | 140 |
| Env 18 | ATHACVPTDPNPQEI | aa 69-83 | 141 |
| Env 19 | CVPTDPNPQEIVLEN | aa 73-87 | 142 |
| Env 20 | DPNPQEIVLENVTEN | aa 77-91 | 143 |
| Env 21 | PQEIVLENVTENFNM | aa 80-94 | 144 |
| Env 22 | VLENVTENFNMWKNN | aa 84-98 | 145 |
| Env 23 | VTENFNMWKNNMVEQ | aa 88-102 | 146 |
| Env 24 | FNMWKNNMVEQMHED | aa 92-106 | 147 |
| Env 25 | KNNMVEQMHEDIISL | aa 96-110 | 148 |
| Env 26 | VEQMHEDIISLWDQS | aa 100-114 | 149 |
| Env 27 | HEDIISLWDQSLEPC | aa 104-118 | 150 |
| Env 28 | ISLWDQSLEPCVKLT | aa 108-122 | 151 |
| Env 29 | DQSLEPCVKLTPLCV | aa 112-126 | 152 |
| Env 30 | EPCVKLTPLCVTLHC | aa 116-130 | 153 |
| Env 31 | KLTPLCVTLHCTNLE | aa 120-134 | 154 |
| Env 32 | LCVTLHCTNLENATN | aa 124-138 | 155 |
| Env 33 | LHCTNLENATNTTSS | aa 128-142 | 156 |
| Env 34 | NLENATNTTSSNWKE | aa 132-146 | 157 |
| Env 35 | ATNTTSSNWKEMNRG | aa 136-150 | 158 |
| Env 36 | TSSNWKEMNRGEIKN | aa 140-154 | 159 |
| Env 37 | WKEMNRGEIKNCSFN | aa 144-158 | 160 |
| Env 38 | NRGEIKNCSFNVTTS | aa 148-162 | 161 |
| Env 39 | IKNCSFNVTTSIGNK | aa 152-166 | 162 |
| Env 40 | SFNVTTSIGNKMQKE | aa 156-170 | 163 |
| Env 41 | TTSIGNKMQKEYALF | aa 160-174 | 164 |
| Env 42 | GNKMQKEYALFYRLD | aa 164-178 | 165 |
| Env 43 | MQKEYALFYRLDVVP | aa 167-181 | 166 |
| Env 44 | YALFYRLDVVPIDND | aa 171-185 | 167 |
| Env 45 | YRLDVVPTDNDNTSY | aa 175-189 | 168 |
| Env 46 | VVPIDNDNTSYNLIN | aa 179-193 | 169 |
| Env 47 | DNDNTSYNLINCNTS | aa 183-197 | 170 |
| Env 48 | TSYNLINCNTSVITQ | aa 187-201 | 171 |
| Env 49 | LINCNTSVITQACPK | aa 191-205 | 172 |
| Env 50 | NTSVITQACPKVSFE | aa 195-209 | 173 |
| Env 51 | ITQACPKVSFEPIPI | aa 199-213 | 174 |
| Env 52 | CPKVSFEPIPIHYCA | aa 203-217 | 175 |
| Env 53 | SFEPIPIHYCAPAGF | aa 207-221 | 176 |
| Env 54 | IPIHYCAPAGFAILK | aa 21-225 | 177 |
| Env 55 | YCAPAGFAILKCNDK | aa 215-229 | 178 |
| Env 56 | AGFAILKCNDKKFNG | aa 219-233 | 179 |
| Env 57 | ILKCNDKKFNGSGPC | aa 223-237 | 180 |
| Env 58 | NDKKFNGSGPCINVS | aa 227-241 | 181 |
| Env 59 | FNGSGPCINVSTVQC | aa 231-245 | 182 |
| Env 60 | GPCINVSTVQCTHGI | aa 235-249 | 183 |
| Env 61 | NVSTVQCTHGIRPVV | aa 239-253 | 184 |
| Env 62 | VQCTHGIRPVVSTQL | aa 243-257 | 185 |
| Env 63 | HGIRPVVSTQLLLNG | aa 247-261 | 186 |
| Env 64 | PVVSTQLLLNGSLAE | aa 251-265 | 187 |
| Env 65 | TQLLLNGSLAEEGVV | aa 255-269 | 188 |
| Env 66 | LNGSLAEEGVVIRSE | aa 259-273 | 189 |
| Env 67 | LAEEGVVIRSENFTD | aa 263-277 | 190 |
| Env 68 | GVVIRSENFTDNVKT | aa 267-281 | 191 |
| Env 69 | RSENFTDNVKTIIVQ | aa 271-285 | 192 |
| Env 70 | FTDNVKTIIVQLKES | aa 275-289 | 193 |
| Env 71 | VKTIIVQLKESVEIN | aa 279-293 | 194 |
| Env 72 | IVQLKESVEINCTRP | aa 283-297 | 195 |
| Env 73 | RESVEINCTRPNNNT | aa 287-301 | 196 |
| Env 74 | EINCTRPNNNTRKSI | aa 291-305 | 197 |
| Env 75 | TRPNNNTRKSIPIGP | aa 295-309 | 198 |
| Env 76 | NNTRKSIPIGPGKAF | aa 299-313 | 199 |
| Env 77 | KSIPIGPGKAFYATG | aa303-317 | 200 |
| Env 78 | IGPGKAFYATGDIIG | aa 307-321 | 201 |
| Env 79 | KAFYATGDIIGDIRQ | aa 311-325 | 202 |
| Env 80 | ATGDIIGDIRQAHCN | aa 315-329 | 203 |
| Env 81 | IIGDIRQAHCNISGE | aa 319-333 | 204 |
| Env 82 | IRQAHCNISGEKWNN | aa 323-337 | 205 |
| Env 83 | HCNISGEKWNNTLKQ | aa 327-341 | 206 |
| Env 84 | SGEKWNNTLKQIVTK | aa 331-345 | 207 |
| Env 85 | NNNTLKQIVTKLQAQ | aa 335-349 | 208 |
| Env 86 | LKQIVTKLQAQFENK | aa 339-353 | 209 |
| Env 87 | VTKLQAQFENKTIVF | aa 343-357 | 210 |
| Env 88 | LQAQFENKTIVFKQS | aa 346-360 | 211 |
| Env 89 | FENXTIVFKQSSGGD | aa350-364 | 212 |
| Env 90 | TIVFKQSSGGDPEIV | aa 354-368 | 213 |
| Env 91 | KQSSGGDPEIVMHSF | aa 358-372 | 214 |
| Env 92 | GGDPEIVMHSFNCGG | aa 362-376 | 215 |
| Env 93 | EIVMHSFNCGGEFFY | aa 366-380 | 216 |
| Env 94 | HSFNCGGEFFYCNST | aa 370-384 | 217 |
| Env 95 | CGGEFFYCNSTQLFN | aa 374-388 | 218 |
| Env 96 | FFYCNSTQLFNSTWN | aa 378-392 | 219 |
| Env 97 | NSTQLFNSTWNNTIG | aa 382-396 | 220 |
| Env 98 | LFNSTWNNTIGPNNT | aa 386-400 | 221 |
| Env 99 | TWNNTIGPNNTNGTI | aa 390-404 | 222 |
| Env 100 | TIGPNNTNGTITLPC | aa 394-408 | 223 |
| Env 101 | NNTNGTITLPCRIKQ | aa 398-412 | 224 |
| Env 102 | GTITLPCRIKQIINR | aa 402-416 | 225 |
| Env 103 | LPCRIKQIINRWQEV | aa 406-420 | 226 |
| Env 104 | IKQIINRWQEVGKAM | aa 410-424 | 227 |
| Env 105 | INRWQEVGKAMYAPP | aa 414-128 | 228 |
| Env 106 | WQEVGKAMYAPPIRG | aa 417-431 | 229 |
| Env 107 | GKAMYAPPIRGQIRC | aa 421-435 | 230 |
| Env 108 | YAPPIRGQIRCSSNI | aa 425-439 | 231 |
| Env 109 | IRGQIRCSSNITGLL | aa 429-443 | 232 |
| Env 110 | IRCSSNITGLLLTRD | aa 433-447 | 233 |
| Env 111 | SNITGLLLTRDGGRE | aa 437-451 | 234 |
| Env 112 | GLLLTRDGGREVGNT | aa 441-455 | 235 |
| Env 113 | TRDGGREVGNTTEIF | aa 445-459 | 236 |
| Env 114 | GREVGNTTEIFRPGG | aa 449-463 | 237 |
| Env 115 | GNTTEIFRPGGGDMR | aa 453-467 | 238 |
| Env 116 | EIFRPGGGDMRDNWR | aa 457-471 | 239 |
| Env 117 | PGGGDMRDNWRSELY | aa 461-475 | 240 |
| Env 118 | DMRDNWRSELYKYKV | aa 465-479 | 241 |
| Env 119 | NWRSELYKYKVVKIE | aa 469-483 | 242 |
| Env 120 | ELYKYKVVKIEPLGV | aa 473-487 | 243 |
| Env 121 | YKVVKIEPLGVAPTK | aa 477-491 | 244 |
| Env 122 | KIEPLGVAPTKAKRR | aa 481-495 | 245 |
| Env 123 | LGVAPTKAKRRVVQR | aa 485-499 | 246 |
| Env 124 | PTKAKRRVVQREKRA | aa 489-503 | 247 |
| Env 125 | KRRVVQREKRAVTLG | aa 493-507 | 248 |
| Env 126 | VQREKRAVTLGAVFL | aa 497-511 | 249 |
| Env 127 | KRAVTLGAVFLGFLG | aa 501-515 | 250 |
| Env 128 | TLGAVFLGFLGAAGS | aa 505-519 | 251 |
| Env 129 | VFLGFLGAAGSTMGA | aa 509-523 | 252 |
| Env 130 | FLGAAGSTMGAASLT | aa 513-527 | 253 |
| Env 131 | AGSTMGAASLTLTVQ | aa 517-531 | 254 |
| Env 132 | MGAASLTLTVQARQL | aa 521-535 | 255 |
| Env 133 | SLTLTVQARQLLSGI | aa 525-539 | 256 |
| Env 8771^{c} | LWVTVYYGVPVWKEA | aa 33-47 | 257 |
| Env 8772^{c} | VYYGVPVWKEATTTL | aa 37-51 | 258 |
| Env 8773^{c} | VPVWKEATTTLFCAS | aa 41-55 | 259 |
| Env 8789^{c} | EDIISLWDQSLKPCV | aa 104-118 | 260 |
| Env 8790^{c} | SLWDQSLKPCVKLTP | aa 108-122 | 261 |
| Env 8791^{c} | QSLKPCVKLTPLCVT | aa 112-126 | 262 |
| Env 8805^{c} | QKEYALFYKLDVVPI | aa 168-182 | 263 |
| Env 8806^{c} | ALFYKLDVVPIDNDN | aa 172-186 | 264 |
| Env 8822^{c} | GPCTNVSTVQCTHGI | aa 235-249 | 265 |

### Mice Immunization.

C57BL/6 mice (H-2^{b}) (Harlan Nossan, Udine, I) were immunized subcutaneously, in a single site in the back, with 25 μg of ovalbumin (Sigma) alone or in combination with native monomeric biologically active Tat protein (5 and 10 μg, respectively) in Freund's adjuvant (CFA for the first injection, and IFA for subsequent injections). BALB/c mice (H-2^{d}) (Harlan, Udine, Italy) were immunized subcutaneously, in a single site in the back, with 5 μg of HIV-1 Gag or Env proteins alone or in combination with 5 μg of native monomeric biologically active Tat protein or with the mutant Tatcys22 protein in Freund's adjuvant or in Alum. Each group was composed of 5 animals. Immunogens were given subcutaneously in 100 μl, at days 1, 14 and 28. Mice were sacrificed 10 days after the last boost (day 38). During the course of the experiments, animals were controlled twice a week at the site of injection and for their general conditions (such as liveliness, food intake, vitality, weight, motility, sheen of hair). No signs of local nor systemic adverse reactions were ever observed in mice receiving the immunogens as compared to control or untreated mice. Animal use was according to European and institutional guidelines.

### Splenocytes Purification.

Splenocytes were purified from spleens squeezed on filters (Cell Strainer, 70 μm, Nylon, Becton Dickinson). Spleens of each experimental group were pooled. Following red blood cell lysis with of 154 mM NH₄Cl, 10 mM KHCO₃ and 0.1 mM EDTA (5 ml/spleen) for 4 minutes at room temperature, cells were diluted with RPMI 1640 containing 3% FBS (Hyclone), spun for 10 minutes at 1200 rpm, resuspended in RPMI 1640 containing 10% FBS and used immediately for the analysis of antigen-specific cellular immune responses (fresh). Cellular responses were also measured after in vitro re-stimulation. Therefore, cells (3-5×10⁶/ml) were stimulated in 14 ml with the Env 10-mer (3 μg/ml), or with pools of Env peptides (15-mers) (10⁻⁶ M) for 5 days before Elispot analysis.

### Cytotoxicity Assay.

Target cells were labelled with Na₂⁵¹CrO₄ for 90 min at 37° C. Cytotoxicity test were routinely run at different effector:target ratios in triplicate. Percent specific lysis was calculated as 100×(cpm sample−cpm medium)/(cpm Triton X-100−cpm medium) (6). Spontaneous release was always less than 20%.

### Elispot Assays.

Elispot (IFN-γ) was carried out using a commercially available kit provided by Becton Dickinson (murine IFNgamma ELISPOT Set; #551083), according to manufacturer's instructions. Briefly, nitrocellulose 96-well plates were coated with 5 μg/ml of anti-IFN-γ overnight at 4° C. The following day, plates were washed 4 times with PBS and blocked with RPMI 1640 supplemented with 10% foetal bovine serum for 2 hours at 37° C. Splenocytes (2.5 and 5×10⁵/200 μl for assays on fresh cells, and 5×10⁴/200 μl for assays on cells in vitro re-stimulated) were added to the wells (duplicate wells) and incubated with peptides (10⁻⁶ M) for 16 hours at 37° C. Controls were represented by cells incubated with Concanavaline A (Sigma; 5 μg/ml) (positive control) or with medium alone (negative control). The spots were read using an Elispot reader (Elivis, Germany). The results are expressed as neat number of spot forming units (SFU)/10⁶ cells: [mean number SFU of peptide treated wells minus mean number SFU of the negative control].

### Results and Discussion

### In Vivo Modulation of Epitope-Specific CTL Responses Against Ovalbumin by the HIV-1 Tat Protein.

We demonstrated in Example 1 that, by altering the antigen processing machinery, Tat influences the number of MHC class I-epitope complexes at the cell surface of antigen presenting cells, thereby modulating CTL responses directed against immunodominant and subdominant epitopes within heterologous antigens. To determine the relevance of these in vitro findings, the effect of Tat on the induction of epitope-specific CTL responses was investigated in vivo.

To this end, K^{b}-restricted CTL responses to ovalbumin (Ova) that are directed to the immunodominant SIINFEKL (SII) epitope (SEQ ID NO 268) and potentially to the subdominant KVVRFDKL (KVV) (SEQ ID NO 269) and the cryptic CFDVFKEL (CFD) (SEQ ID NO 270) epitopes (7, 8) were used as model systems. In fact, it has been shown that KVV- and CFD-specific CTLs are not found upon immunization of C57BL/6 mice with Ova and that the lack of these responses is due to a poor generation of KVV and CFD epitopes by proteasomes (9, 10).

To address the generation of CTL responses to the K^{b}-restricted Ova-derived epitopes, C57BL/6 mice were vaccinated with Ova alone or in combination with the Tat protein. We then evaluated the presence of epitope-specific CTL responses in fresh splenocytes that were tested against EL4 target cells pulsed with the relevant peptides (FIG. 20). Splenocytes isolated from mice immunized with Ova alone recognised target cells pulsed with the SII epitope but did not recognise cells pulsed with the KVV or CFD epitopes, confirming that CTL responses are mainly directed against the immunodominant SII peptide epitope. In contrast, splenocytes isolated from mice vaccinated with the combination Ova/Tat recognized less efficiently the immunodominant SII epitope, whereas clearly recognised target cells presenting the subdominant KVV and the cryptic CFD epitopes, respectively. Control mice did not recognise any peptide-pulsed EL4 cells.

### In Vivo Modulation of Epitope-Specific T Cell Responses Against Env and Gag by the HIV-1 Tat Protein.

To address the effect of Tat on epitope specific T cell responses directed to Gag and Env antigens, BALB/c mice were vaccinated with the HIV-1 Env or HIV-1 Gag proteins alone, or in combination with the Tat protein. The presence of peptide-specific T cell responses was evaluated by IFN-γ Elispot assays using fresh splenocytes stimulated with pools of peptides spanning the entire sequence of Env and Gag proteins.

Fresh splenocytes isolated from mice immunized with Env, either alone or in combination with Tat, did not respond to stimulation with any of the Env-derived peptide pools (data not shown). Subsequently, splenocytes from Env-vaccinated mice were stimulated with the immunodominant K^{d}-restricted RGP CTL epitope (amino acid 311-320), or with pools of peptides covering the entire Env sequence. After 5 days, all cultures were tested for specificity by IFN-γ Elispot following stimulation with pools of peptides using a peptide-based matrix approach. Matrices consisted of pools of peptides in which each peptide was present in two separate pools (see FIG. 21).

As shown in FIG. 16, after immunization with Env alone, IFNγ responses were detected against Env pools 1, 5, 16, 17, 21, 22, 25, and 26. Similarly, after immunization with Tat+Env and with TatCys22+Env, an IFNγ responses was detected against the same Env pools 1, 5, 16, 17, 21, 22, 25, and 26. However, in the presence of Tat wild-type and TatCys22, additional responses to Env pools 4, 7, 14, 15, 18, 19, 23 and 27 were detected.

These results indicate that Tat and TatCys22 generally broaden the immune response to Env.

Fresh splenocytes isolated from mice immunized, with Gag alone or with Gag and Tat, were stimulated with pools of peptides using a peptide-based matrix approach and assayed by IFN-γ Elispot. Matrices consisted of pools of peptides in which each peptide was present in two separate pools (see FIG. 22), thus offering internal positive controls.

Responses were regarded as positive if they had at least three times the mean number of SFU in the control wells, and had to be ≧50 SFU/10⁶ cells.

As shown in FIG. 17, mice immunized with Gag alone responded to pools 5, 6, 9, 10, 15, 16, 17 and 18, whereas mice immunized with both Gag and Tat responded to pools 3, 5, 6, 9, 10, 13, 15, 16, 18 and 19. Control mice did not respond to any of the pools (not shown). In previous studies it has been demonstrated that major K^{d}-restricted CTL responses to Gag are directed to AMQ peptide (amino acid 197-205) contained in pools 5, 6 and 16, and to TTS peptide (amino acid 239-247) contained in pools 4, 5, and 17.

Interestingly, splenocytes from mice vaccinated with Tat and Gag, as compared to mice vaccinated with Gag alone, did not respond to pool 17 (containing the TTS peptide), whereas they recognized pools 13 and 19. We then assayed 36 individual peptides identified as potential targets by the matrix approach. As shown in FIG. 18, splenocytes from mice immunized with Gag alone responded to 6 different peptides (Gag42, Gag49, Gag50, Gag65, Gag75, Gag76), four of which (Gag49 and Gag50; Gag75 and Gag76) may contain 2 different overlapping peptides suggesting that T cell responses induced by Gag vaccination are directed to 4 different T cell epitopes. In contrast, mice immunized with Gag and Tat responded to 7 different peptides (Gag20, Gag39, Gag42, Gag49, Gag69, Gag76, Gag80) suggesting that T cell responses induced by Gag+Tat vaccination are directed to 7 different T cell epitopes, three more than vaccination with Gag alone.

### In Vivo Modulation of Epitope-Specific T Cell Responses Against Env and Gag by a Mutated Tat Protein (Tatcys22).

In the next set of experiments we evaluated the effect a Tat mutant carrying a glycine at position 22 instead of cysteine (referred to as Tatcys22). Tatcys22, in contrast to wild-type Tat, has no effect on the transactivation of the HIV-1 LTR, and does not induce reactivation of latent infection.

To address the effect of Tatcys22 on epitope specific T cell responses directed to Gag, BALB/c mice were also vaccinated with HIV-1 Gag protein alone, or in combination with the Tatcys22 protein, and assayed as previously described.

As shown in FIG. 19, splenocytes isolated from mice immunized with Gag and Tatcys22 recognised more peptide pools than splenocytes from mice immunized with Gag alone. In particular, Tatcys22 induces Gag-specific responses directed to pools 3, 8, 13 and 19, which were not recognized after immunization with Gag alone. As previously, we then assayed 36 individual peptides (FIG. 20) identified by the matrix approach and we found that mice immunized with Gag in combination with Tatcys22 recognised 16 different peptides (Gag20, Gag21, Gag39, Gag42, Gag49, Gag50, Gag53, Gag60, Gag61, Gag64, Gag65, Gag69, Gag74, Gag75, Gag76, Gag80), 10 of which (Gag20 and Gag21; Gag49 and Gag50; Gag60 and Gag61; Gag64 and 65; Gag75 and Gag76) may contain 5 different overlapping peptides, suggesting that T cell responses induced by vaccination with Gag+Tatcys22 are directed to 11 different T cell epitopes, 7 more than mice immunized with Gag alone, and 4 more than mice immunized with Gag and wild-type Tat.

### Conclusions

We have shown that native HIV-1 Tat protein and the mutant Tatcys22 protein modulate in vivo epitope specific T cell responses to the HIV-1 Gag and Env antigens. In particular, we have demonstrated that mice vaccinated with Gag, in combination with wild-type Tat or with the mutant Tatcys22, responded to 7 or 11 T cell Gag-derived epitopes respectively, in contrast to mice vaccinated with Gag alone, which responded to 4 T cell Gag-derived epitopes. Similarly, mice vaccinated with Env, in combination with wild-type Tat or with the mutant Tatcys22 responded to 12 Env-derived pools of peptides epitopes in contrast to mice vaccinated with Env alone which responded to 8 T cell Env-derived peptide pools.

These observations, together with our previous findings (2, 3, 11, 12), suggest that Tat is not only an antigen but also a novel adjuvant capable of increasing T cell responses against heterologous antigens. Therefore, the Tat protein, as well as mutant Tatcys22, may represent an important tool in HIV-1 vaccine strategies aimed at broadening the spectrum of the epitopes recognized by T cells.

### REFERENCES FOR EXAMPLE 3

1. Wu, Y., and J. W. Marsh. 2003. Gene transcription in HIV infection. Microbes Infect. 5:1023.
2. Fanales-Belasio, E., S. Moretti, F. Nappi, G. Barillari, F. Micheletti, A. Cafaro, and B. Ensoli. 2002. Native HIV-1 Tat protein is selectively taken up by monocyte-derived dendritic cells and induces their maturation, Th-1 cytokine production and antigen presenting function. J. Immunol. 168:197.
3. Gavioli, R., E. Gallerani, C. Fortini, M. Fabris, A. Bottoni, A. Canella, A. Bonaccorsi, M. Marastoni, F. Micheletti, A. Cafaro, P. Rimessi, A. Caputo, and B. Ensoli. 2004. HIV-1 Tat protein modulates cytotoxic T cell epitopes by modifying proteasome composition and enzymatic activity. J. Immunol. 173:3838.
4. Ensoli, B., L. Buonaguro, G. Barillari, V. Fiorelli; R. Gendelman, R. A. Morgan, P. Wingfield, and R. C. Gallo. 1993. Release, uptake, and effect of extracellular human immunodeficiency virus type 1 Tat protein on cell growth and viral transactivation. J. Virol. 67:277.
5. Micheletti, F., A. Canella, S. Vertuani, M. Marastoni, L. Tosi, S. Volinia, S. Traniello, and R. Gavioli. 2000. Supra-agonist peptides enhance the reactivation of memory cytotoxic T lymphocyte responses. J. Immunol. 165:4264.
6. Gavioli, R., M. G. Kurilla, P. O. de Campos-Lima, L. E. Wallace, R. Dolcetti, R. J. Murray, A. B. Rickinson, and M. G. Masucci. 1993. Multiple HLA A11-restricted cytotoxic T-lymphocyte epitopes of different immunogenicities in the Epstein-Barr virus-encoded nuclear antigen 4. J. Virol. 67:1572.
7. Chen, W., S. Khilko, J. Fecondo, D. H. Margulies, and J. McCluskey. 1994. Determinant selection of major histocompatibility complex class I-restricted antigenic peptides is explained by class I-peptide affinity and is strongly influenced by nondominant anchor residues. J. Exp. Med. 180:1471.
8. Lipford, G. B., M. Hoffman, H. Wagner, and K. Heeg. 1993. Primary in vivo responses to ovalbumin. J. Immunol. 150:1212.
9. Niedermann, G., S. Butz, H. G. Ihlenfeldt, R. Grimm, M. Lucchiari, H. Hoschützky, G. Jung, B. Maier, and K. Eichmann. 1995. Contribution of proteasome-mediated proteolysis to the hierarchy of epitopes presented by major histocompatibility complex class I molecules. Immunity 2:289.
10. Mo, A. X. Y., S. F. L. van Lelyveld, A. Craiu, and K. L. Rock. 2000. Sequences that flank subdominant and cryptic epitopes influence the proteolytic generation of MHC class I-presented peptides. J. Immunol. 164:4003.
11. Cafaro, A., A. Caputo, C. Fracasso, M. T. Maggiorella, D. Goletti, S. Baroncelli, M. Pace, L. Semicola, M. L. Koanga-Mogtomo, M. Betti, A. Borsetti, R. Belli, L. Åkerblom, F. Corrias, S. Butto, J. Heeney, P. Verani, F. Titti, and B. Ensoli. 1999. Control of SHIV-89.6P-infection of cynomolgus monkeys by HIV-1 Tat protein vaccine. Nat. Med. 5:643.
12. Cafaro, A., F. Titti, C. Fracasso, M. T. Maggiorella, S. Baroncelli, A. Caputo, D. Goletti, A. Borsetti, M. Pace, E. Fanales-Belasio, B. Ridolfi, D. R. Negri, L. Semicola, R. Belli, F. Corrias, I. Macchia, P. Leone, Z. Michelini, P. ten Haaft, S. Butto, P. Verani, and B. Ensoli. 2001. Vaccination with DNA containing tat coding sequences and unmethylated CpG motifs protects cynomolgus monkeys upon infection with simian/human immunodeficiency virus (SHIV89.6P). Vaccine 19:2862.

1. Use of Tat, a biologically active equivalent, or a precursor therefor, in the preparation of a vaccine suitable to elicit an immune response against both sub-dominant epitopes and immunodominant epitopes from an antigenic substance having a plurality of epitopes, including both immunodominant and sub-dominant epitopes, the vaccine comprising at least a part of the antigenic substance encoding or comprising a sub-dominant epitope thereof.

2. Use of Tat, a biologically active equivalent, thereof or a precursor therefor, in the preparation of a vaccine suitable to elicit an immune response against both sub-dominant epitopes and immunodominant epitopes from a plurality of strains of an infectious organism, the vaccine comprising antigenic material from at least one strain of the organism, said material encoding or comprising a sub-dominant epitope.

3. Use according to claim 1, wherein the sub-dominant epitope is a cryptic epitope.

4. Use according to claim 1, wherein Tat is that shown in SEQ ID NO. 284.

5. Use according to claim 1, wherein Tat is a mutant and/or is a fragment of that shown in SEQ ID No. 284.

6. Use according to claim 5, wherein the mutant Tat has 90% homology to SEQ ID NO. 284.

7. Use according to claim 5, wherein Tat is mutated at position 22 of SEQ ID NO. 284.

8. Use according to claim 7, wherein a Cysteine residue is present at position 22 and is substituted by glycine.

9. Use according claim 1, wherein a fragment of Tat is used, comprising or encoding amino acid numbers 47-86 of SEQ ID NO 284.

10. Use according to claim 9, wherein the fragment is a polypeptide consisting of amino acid numbers 47-86 of SEQ ID NO 284.

11. Use according to claim 1, wherein the vaccine comprises an expression sequence for Tat together with a vector therefor, said expression sequence being suitable to express Tat in a target cell.

12. Use according to claim 11, wherein Tat is under the control of an inducible promoter.

13. Use according to any claim 1, wherein Tat is provided as a peptide or protein.

14. Use according to claim 1, wherein the antigen is derived from or comprises HIV.

15. Use according to claim 1, wherein the antigen is derived from or comprises influenza or SARS.

16. Use according to claim 1, wherein the antigenic substance is associated with a tumour or immunomediated disease.

17. Use according to claim 1, wherein the antigen is derived from plants, parasites, fungi, bacteria or viruses.

18. Use according to claim 16, wherein the antigen is derived from or comprises intracellular pathogens.

19. Use according to claim 14, wherein the peptide is derived from or comprises Gag, or a fragment thereof.

20. Use according to claim 14, wherein the peptide is derived from or comprises Env, or a fragment thereof.

21. Use according to claim 1, wherein the vaccine is administered orally, intravenously, intramuscularly, intraperitonealy, transdermally, or subcutaneously.

22. Use according to claim 1, wherein the vaccine is prime-boost regimen.

23. Use according to claim 1, wherein the Tat, its equivalent, or precursor, is capable of down-regulating levels of LMP2 in the intended recipient of the vaccine.

24. Use of a vaccine for modulating proteosome subunit composition, by administering Tat to down-regulate expression of the LMP2 subunit.

25. A vaccine for eliciting an immune response against both sub-dominant epitopes and immunodominant epitopes from an antigenic substance having a plurality of epitopes, the vaccine comprising Tat, a biologically active equivalent, or a precursor therefor, the epitopes including both immunodominant and sub-dominant epitopes, and at least a part of the antigenic substance encoding or comprising a sub-dominant epitope thereof.

26. A vaccine for eliciting an immune response against both sub-dominant epitopes and immunodominant epitopes from a plurality of strains of an infectious organism, the vaccine comprising Tat, a biologically active equivalent, thereof or a precursor therefore, and antigenic material from at least one strain of the organism, said material encoding or comprising a subdominant epitope.

27. The vaccine according to claim 25, wherein the Tat is that shown in SEQ ID NO. 284.

28. The vaccine according to claim 25, wherein sub-dominant epitope is a cryptic epitope.

29. The vaccine for use in claim 1.

30. The vaccine according claim 25, wherein Tat and the antigen are provided as proteins or peptides.

31. Use of the vaccine according to claim 25 to stimulate cross-strain immunity.

32. A vaccine comprising Tat and an antigen, as defined in claim 1, and a vehicle therefor.

33. A method for providing an immune response against a plurality of strains of an infectious organism, comprising administering a vaccine comprising:
antigenic material from at least one strain of the organism, said material encoding or comprising a subdominant epitope; and
Tat, a biologically active equivalent, thereof or a precursor therefor.

34. The method according to claim 33, wherein the Tat is a mutant or fragment of SEQ ID NO 284.


SEQUENCE LISTING (290 sequences)

SEQ ID NO: 1 - protein, 15 aa, Artificial
  Description of Artificial Sequence Synthetic peptide
MGARASVLSGGELDR

SEQ ID NO: 2 - protein, 15 aa, Artificial
  Description of Artificial Sequence Synthetic peptide
ASVLSGGELDRWEKI

SEQ ID NO: 3 - protein, 15 aa, Artificial
  Description of Artificial Sequence Synthetic peptide
SGGELDRWEKIRLRP

SEQ ID NO: 4 - protein, 15 aa, Artificial
  Description of Artificial Sequence Synthetic peptide
LDRWEKIRLRPGGKK

SEQ ID NO: 5 - protein, 15 aa, Artificial
  Description of Artificial Sequence Synthetic peptide
EKIRLRPGGKKKYKL

SEQ ID NO: 6 - protein, 15 aa, Artificial
  Description of Artificial Sequence Synthetic peptide
LRPGGKKKYKLKHIV

SEQ ID NO: 7 - protein, 15 aa, Artificial
  Description of Artificial Sequence Synthetic peptide
GKKKYKLKHIVWASR

SEQ ID NO: 8 - protein, 15 aa, Artificial
  Description of Artificial Sequence Synthetic peptide
YKLKHIVWASRELER

SEQ ID NO: 9 - protein, 15 aa, Artificial
  Description of Artificial Sequence Synthetic peptide
HIVWASRELERFAVN

SEQ ID NO: 10 - protein, 15 aa, Artificial
  Description of Artificial Sequence Synthetic peptide
ASRELERFAVNPGLL

SEQ ID NO: 11 - protein, 15 aa, Artificial
  Description of Artificial Sequence Synthetic peptide
LERFAVNPGLLETSE

SEQ ID NO: 12 - protein, 15 aa, Artificial
  Description of Artificial Sequence Synthetic peptide
AVNPGLLETSEGCRQ

SEQ ID NO: 13 - protein, 15 aa, Artificial
  Description of Artificial Sequence Synthetic peptide
GLLETSEGCRQILGQ

SEQ ID NO: 14 - protein, 15 aa, Artificial
  Description of Artificial Sequence Synthetic peptide
TSEGCRQILGQLQPS

SEQ ID NO: 15 - protein, 15 aa, Artificial
  Description of Artificial Sequence Synthetic peptide
CRQILGQLQPSLQTG

SEQ ID NO: 16 - protein, 15 aa, Artificial
  Description of Artificial Sequence Synthetic peptide
LGQLQPSLQTGSEEL

SEQ ID NO: 17 - protein, 15 aa, Artificial
  Description of Artificial Sequence Synthetic peptide
QPSLQTGSEELRSLY

SEQ ID NO: 18 - protein, 15 aa, Artificial
  Description of Artificial Sequence Synthetic peptide
QTGSEELRSLYNTVA

SEQ ID NO: 19 - protein, 15 aa, Artificial
  Description of Artificial Sequence Synthetic peptide
EELRSLYNTVATLYC

SEQ ID NO: 20 - protein, 15 aa, Artificial
  Description of Artificial Sequence Synthetic peptide
SLYNTVATLYCVHQR

SEQ ID NO: 21 - protein, 15 aa, Artificial
  Description of Artificial Sequence Synthetic peptide
TVATLYCVHQRIEVK

SEQ ID NO: 22 - protein, 15 aa, Artificial
  Description of Artificial Sequence Synthetic peptide
LYCVHQRIEVKDTKE

SEQ ID NO: 23 - protein, 15 aa, Artificial
  Description of Artificial Sequence Synthetic peptide
HQRIEVKDTKEALEK

SEQ ID NO: 24 - protein, 15 aa, Artificial
  Description of Artificial Sequence Synthetic peptide
EVKDTKEALEKIEEE

SEQ ID NO: 25 - protein, 15 aa, Artificial
  Description of Artificial Sequence Synthetic peptide
TKEALEKIEEEQNKS

SEQ ID NO: 26 - protein, 15 aa, Artificial
  Description of Artificial Sequence Synthetic peptide
LEKIEEEQNKSKKKA

SEQ ID NO: 27 - protein, 15 aa, Artificial
  Description of Artificial Sequence Synthetic peptide
EEEQNKSKKKAQQAA

SEQ ID NO: 28 - protein, 15 aa, Artificial
  Description of Artificial Sequence Synthetic peptide
NKSKKKAQQAAADTG

SEQ ID NO: 29 - protein, 15 aa, Artificial
  Description of Artificial Sequence Synthetic peptide
KKAQQAAADTGNSSQ

SEQ ID NO: 30 - protein, 15 aa, Artificial
  Description of Artificial Sequence Synthetic peptide
QAAADTGNSSQVSQN

SEQ ID NO: 31 - protein, 15 aa, Artificial
  Description of Artificial Sequence Synthetic peptide
DTGNSSQVSQNYPIV

SEQ ID NO: 32 - protein, 15 aa, Artificial
  Description of Artificial Sequence Synthetic peptide
SSQVSQNYPIVQNLQ

SEQ ID NO: 33 - protein, 15 aa, Artificial
  Description of Artificial Sequence Synthetic peptide
SQNYPIVQNLQGQMV

SEQ ID NO: 34 - protein, 15 aa, Artificial
  Description of Artificial Sequence Synthetic peptide
PIVQNLQGQMVHQAI

SEQ ID NO: 35 - protein, 15 aa, Artificial
  Description of Artificial Sequence Synthetic peptide
NLQGQMVHQAISPRT

SEQ ID NO: 36 - protein, 15 aa, Artificial
  Description of Artificial Sequence Synthetic peptide
QMVHQAISPRTLNAW

SEQ ID NO: 37 - protein, 15 aa, Artificial
  Description of Artificial Sequence Synthetic peptide
QAISPRTLNAWVKVV

SEQ ID NO: 38 - protein, 15 aa, Artificial
  Description of Artificial Sequence Synthetic peptide
PRTLNAWVKVVEEKA

SEQ ID NO: 39 - protein, 15 aa, Artificial
  Description of Artificial Sequence Synthetic peptide
NAWVKVVEEKAFSPE

SEQ ID NO: 40 - protein, 15 aa, Artificial
  Description of Artificial Sequence Synthetic peptide
KVVEEKAFSPEVIPM

SEQ ID NO: 41 - protein, 15 aa, Artificial
  Description of Artificial Sequence Synthetic peptide
EKAFSPEVIPMFSAL

SEQ ID NO: 42 - protein, 15 aa, Artificial
  Description of Artificial Sequence Synthetic peptide
SPEVIPMFSALSEGA

SEQ ID NO: 43 - protein, 15 aa, Artificial
  Description of Artificial Sequence Synthetic peptide
IPMFSALSEGATPQD

SEQ ID NO: 44 - protein, 15 aa, Artificial
  Description of Artificial Sequence Synthetic peptide
SALSEGATPQDLNTM

SEQ ID NO: 45 - protein, 15 aa, Artificial
  Description of Artificial Sequence Synthetic peptide
EGATPQDLNTMLNTV

SEQ ID NO: 46 - protein, 15 aa, Artificial
  Description of Artificial Sequence Synthetic peptide
PQDLNTMLNTVGGHQ

SEQ ID NO: 47 - protein, 15 aa, Artificial
  Description of Artificial Sequence Synthetic peptide
NTMLNTVGGHQAAMQ

SEQ ID NO: 48 - protein, 15 aa, Artificial
  Description of Artificial Sequence Synthetic peptide
NTVGGHQAAMQMLKE

SEQ ID NO: 49 - protein, 15 aa, Artificial
  Description of Artificial Sequence Synthetic peptide
GHQAAMQMLKETINE

SEQ ID NO: 50 - protein, 15 aa, Artificial
  Description of Artificial Sequence Synthetic peptide
AMQMLKETINEEAAE

SEQ ID NO: 51 - protein, 15 aa, Artificial
  Description of Artificial Sequence Synthetic peptide
LKETINEEAAEWDRL

SEQ ID NO: 52 - protein, 15 aa, Artificial
  Description of Artificial Sequence Synthetic peptide
INEEAAEWDRLHPVH

SEQ ID NO: 53 - protein, 15 aa, Artificial
  Description of Artificial Sequence Synthetic peptide
AAEWDRLHPVHAGPI

SEQ ID NO: 54 - protein, 15 aa, Artificial
  Description of Artificial Sequence Synthetic peptide
DRLHPVHAGPIAPGQ

SEQ ID NO: 55 - protein, 15 aa, Artificial
  Description of Artificial Sequence Synthetic peptide
PVHAGPIAPGQMREP

SEQ ID NO: 56 - protein, 15 aa, Artificial
  Description of Artificial Sequence Synthetic peptide
GPIAPGQMREPRGSD

SEQ ID NO: 57 - protein, 15 aa, Artificial
  Description of Artificial Sequence Synthetic peptide
PGQMREPRGSDIAGT

SEQ ID NO: 58 - protein, 15 aa, Artificial
  Description of Artificial Sequence Synthetic peptide
REPRGSDIAGTTSTL

SEQ ID NO: 59 - protein, 15 aa, Artificial
  Description of Artificial Sequence Synthetic peptide
GSDIAGTTSTLQEQI

SEQ ID NO: 60 - protein, 15 aa, Artificial
  Description of Artificial Sequence Synthetic peptide
AGTTSTLQEQIGWMT

SEQ ID NO: 61 - protein, 15 aa, Artificial
  Description of Artificial Sequence Synthetic peptide
STLQEQIGWMTNNPP

SEQ ID NO: 62 - protein, 15 aa, Artificial
  Description of Artificial Sequence Synthetic peptide
EQIGWMTNNPPIPVG

SEQ ID NO: 63 - protein, 15 aa, Artificial
  Description of Artificial Sequence Synthetic peptide
WMTNNPPIPVGEIYK

SEQ ID NO: 64 - protein, 15 aa, Artificial
  Description of Artificial Sequence Synthetic peptide
NPPIPVGEIYKRWII

SEQ ID NO: 65 - protein, 15 aa, Artificial
  Description of Artificial Sequence Synthetic peptide
PVGEIYKRWIILGLN

SEQ ID NO: 66 - protein, 15 aa, Artificial
  Description of Artificial Sequence Synthetic peptide
IYKRWIILGLNKIVR

SEQ ID NO: 67 - protein, 15 aa, Artificial
  Description of Artificial Sequence Synthetic peptide
WIILGLNKIVRMYSP

SEQ ID NO: 68 - protein, 15 aa, Artificial
  Description of Artificial Sequence Synthetic peptide
GLNKIVRMYSPTSIL

SEQ ID NO: 69 - protein, 15 aa, Artificial
  Description of Artificial Sequence Synthetic peptide
IVRMYSPTSILDIRQ

SEQ ID NO: 70 - protein, 15 aa, Artificial
  Description of Artificial Sequence Synthetic peptide
YSPTSILDIRQGPKE

SEQ ID NO: 71 - protein, 15 aa, Artificial
  Description of Artificial Sequence Synthetic peptide
SILDIRQGPKEPFRD

SEQ ID NO: 72 - protein, 15 aa, Artificial
  Description of Artificial Sequence Synthetic peptide
IRQGPKEPFRDYVDR

SEQ ID NO: 73 - protein, 15 aa, Artificial
  Description of Artificial Sequence Synthetic peptide
PKEPFRDYVDRFYKT

SEQ ID NO: 74 - protein, 15 aa, Artificial
  Description of Artificial Sequence Synthetic peptide
FRDYVDRFYKTLRAE

SEQ ID NO: 75 - protein, 15 aa, Artificial
  Description of Artificial Sequence Synthetic peptide
VDRFYKTLRAEQASQ

SEQ ID NO: 76 - protein, 15 aa, Artificial
  Description of Artificial Sequence Synthetic peptide
YKTLRAEQASQEVKN

SEQ ID NO: 77 - protein, 15 aa, Artificial
  Description of Artificial Sequence Synthetic peptide
RAEQASQEVKNWMTE

SEQ ID NO: 78 - protein, 15 aa, Artificial
  Description of Artificial Sequence Synthetic peptide
ASQEVKNWMTETLLV

SEQ ID NO: 79 - protein, 15 aa, Artificial
  Description of Artificial Sequence Synthetic peptide
VKNWMTETLLVQNAN

SEQ ID NO: 80 - protein, 15 aa, Artificial
  Description of Artificial Sequence Synthetic peptide
MTETLLVQNANPDCK

SEQ ID NO: 81 - protein, 15 aa, Artificial
  Description of Artificial Sequence Synthetic peptide
LLVQNANPDCKTILK

SEQ ID NO: 82 - protein, 15 aa, Artificial
  Description of Artificial Sequence Synthetic peptide
NANPDCKTILKALGP

SEQ ID NO: 83 - protein, 15 aa, Artificial
  Description of Artificial Sequence Synthetic peptide
DCKTILKALGPAATL

SEQ ID NO: 84 - protein, 15 aa, Artificial
  Description of Artificial Sequence Synthetic peptide
ILKALGPAATLEEMM

SEQ ID NO: 85 - protein, 15 aa, Artificial
  Description of Artificial Sequence Synthetic peptide
LGPAATLEEMMTACQ

SEQ ID NO: 86 - protein, 15 aa, Artificial
  Description of Artificial Sequence Synthetic peptide
ATLEEMMTACQGVGG

SEQ ID NO: 87 - protein, 15 aa, Artificial
  Description of Artificial Sequence Synthetic peptide
EMMTACQGVGGPGHK

SEQ ID NO: 88 - protein, 15 aa, Artificial
  Description of Artificial Sequence Synthetic peptide
ACQGVGGPGHKARVL

SEQ ID NO: 89 - protein, 15 aa, Artificial
  Description of Artificial Sequence Synthetic peptide
VGGPGHKARVLAEAM

SEQ ID NO: 90 - protein, 15 aa, Artificial
  Description of Artificial Sequence Synthetic peptide
GHKARVLAEAMSQVT

SEQ ID NO: 91 - protein, 15 aa, Artificial
  Description of Artificial Sequence Synthetic peptide
RVLAEAMSQVTNSAT

SEQ ID NO: 92 - protein, 15 aa, Artificial
  Description of Artificial Sequence Synthetic peptide
EAMSQVTNSATIMMQ

SEQ ID NO: 93 - protein, 15 aa, Artificial
  Description of Artificial Sequence Synthetic peptide
QVTNSATIMMQRGNF

SEQ ID NO: 94 - protein, 15 aa, Artificial
  Description of Artificial Sequence Synthetic peptide
SATIMMQRGNFRNQR

SEQ ID NO: 95 - protein, 15 aa, Artificial
  Description of Artificial Sequence Synthetic peptide
MMQRGNFRNQRKTVK

SEQ ID NO: 96 - protein, 15 aa, Artificial
  Description of Artificial Sequence Synthetic peptide
GNFRNQRKTVKCFNC

SEQ ID NO: 97 - protein, 15 aa, Artificial
  Description of Artificial Sequence Synthetic peptide
NQRKTVKCFNCGKEG

SEQ ID NO: 98 - protein, 15 aa, Artificial
  Description of Artificial Sequence Synthetic peptide
TVKCFNCGKEGHIAK

SEQ ID NO: 99 - protein, 15 aa, Artificial
  Description of Artificial Sequence Synthetic peptide
FNCGKEGHIAKNCRA

SEQ ID NO: 100 - protein, 15 aa, Artificial
  Description of Artificial Sequence Synthetic peptide
KEGHIAKNCRAPRKK

SEQ ID NO: 101 - protein, 15 aa, Artificial
  Description of Artificial Sequence Synthetic peptide
IAKNCRAPRKKGCWK

SEQ ID NO: 102 - protein, 15 aa, Artificial
  Description of Artificial Sequence Synthetic peptide
CRAPRKKGCWKCGKE

SEQ ID NO: 103 - protein, 15 aa, Artificial
  Description of Artificial Sequence Synthetic peptide
RKKGCWKCGKEGHQM

SEQ ID NO: 104 - protein, 15 aa, Artificial
  Description of Artificial Sequence Synthetic peptide
CWKCGKEGHQMKDCT

SEQ ID NO: 105 - protein, 15 aa, Artificial
  Description of Artificial Sequence Synthetic peptide
GKEGHQMKDCTERQA

SEQ ID NO: 106 - protein, 15 aa, Artificial
  Description of Artificial Sequence Synthetic peptide
HQMKDCTERQANFLG

SEQ ID NO: 107 - protein, 15 aa, Artificial
  Description of Artificial Sequence Synthetic peptide
DCTERQANFLGKIWP

SEQ ID NO: 108 - protein, 15 aa, Artificial
  Description of Artificial Sequence Synthetic peptide
RQANFLGKIWPSHKG

SEQ ID NO: 109 - protein, 15 aa, Artificial
  Description of Artificial Sequence Synthetic peptide
FLGKIWPSHKGRPGN

SEQ ID NO: 110 - protein, 15 aa, Artificial
  Description of Artificial Sequence Synthetic peptide
IWPSHKGRPGNFLQS

SEQ ID NO: 111 - protein, 15 aa, Artificial
  Description of Artificial Sequence Synthetic peptide
HKGRPGNFLQSRPEP

SEQ ID NO: 112 - protein, 15 aa, Artificial
  Description of Artificial Sequence Synthetic peptide
PGNFLQSRPEPTAPP

SEQ ID NO: 113 - protein, 15 aa, Artificial
  Description of Artificial Sequence Synthetic peptide
LQSRPEPTAPPEESF

SEQ ID NO: 114 - protein, 15 aa, Artificial
  Description of Artificial Sequence Synthetic peptide
PEPTAPPEESFRFGE

SEQ ID NO: 115 - protein, 15 aa, Artificial
  Description of Artificial Sequence Synthetic peptide
APPEESFRFGEETTT

SEQ ID NO: 116 - protein, 15 aa, Artificial
  Description of Artificial Sequence Synthetic peptide
ESFRFGEETTTPSQK

SEQ ID NO: 117 - protein, 15 aa, Artificial
  Description of Artificial Sequence Synthetic peptide
FGEETTTPSQKQEPI

SEQ ID NO: 118 - protein, 15 aa, Artificial
  Description of Artificial Sequence Synthetic peptide
TTTPSQKQEPIDKEL

SEQ ID NO: 119 - protein, 15 aa, Artificial
  Description of Artificial Sequence Synthetic peptide
SQKQEPIDKELYPLA

SEQ ID NO: 120 - protein, 15 aa, Artificial
  Description of Artificial Sequence Synthetic peptide
EPIDKELYPLASLRS

SEQ ID NO: 121 - protein, 15 aa, Artificial
  Description of Artificial Sequence Synthetic peptide
KELYPLASLRSLFGN

SEQ ID NO: 122 - protein, 15 aa, Artificial
  Description of Artificial Sequence Synthetic peptide
PLASLRSLFGNDPSS

SEQ ID NO: 123 - protein, 12 aa, Artificial
  Description of Artificial Sequence Synthetic peptide
LRSLFGNDPSSQ

SEQ ID NO: 124 - protein, 15 aa, Artificial
  Description of Artificial Sequence Synthetic peptide
MRVKGIRKNYQHLWR

SEQ ID NO: 125 - protein, 15 aa, Artificial
  Description of Artificial Sequence Synthetic peptide
GIRKNYQHLWRGGTL

SEQ ID NO: 126 - protein, 15 aa, Artificial
  Description of Artificial Sequence Synthetic peptide
NYQHLWRGGTLLLGM

SEQ ID NO: 127 - protein, 15 aa, Artificial
  Description of Artificial Sequence Synthetic peptide
LWRGGTLLLGMLMIC

SEQ ID NO: 128 - protein, 15 aa, Artificial
  Description of Artificial Sequence Synthetic peptide
GTLLLGMLMICSAVE

SEQ ID NO: 129 - protein, 15 aa, Artificial
  Description of Artificial Sequence Synthetic peptide
LGMLMICSAVEKLWV

SEQ ID NO: 130 - protein, 15 aa, Artificial
  Description of Artificial Sequence Synthetic peptide
MICSAVEKLWVTVYY

SEQ ID NO: 131 - protein, 15 aa, Artificial
  Description of Artificial Sequence Synthetic peptide
AVEKLWVTVYYGVPA

SEQ ID NO: 132 - protein, 15 aa, Artificial
  Description of Artificial Sequence Synthetic peptide
LWVTVYYGVPAWKEA

SEQ ID NO: 133 - protein, 15 aa, Artificial
  Description of Artificial Sequence Synthetic peptide
VYYGVPAWKEATTTL

SEQ ID NO: 134 - protein, 15 aa, Artificial
  Description of Artificial Sequence Synthetic peptide
VPAWKEATTTLFCAS

SEQ ID NO: 135 - protein, 15 aa, Artificial
  Description of Artificial Sequence Synthetic peptide
KEATTTLFCASDAKA

SEQ ID NO: 136 - protein, 15 aa, Artificial
  Description of Artificial Sequence Synthetic peptide
TTLFCASDAKAYDTE

SEQ ID NO: 137 - protein, 15 aa, Artificial
  Description of Artificial Sequence Synthetic peptide
CASDAKAYDTEVHNV

SEQ ID NO: 138 - protein, 15 aa, Artificial
  Description of Artificial Sequence Synthetic peptide
AKAYDTEVHNVWATH

SEQ ID NO: 139 - protein, 15 aa, Artificial
  Description of Artificial Sequence Synthetic peptide
DTEVHNVWATHACVP

SEQ ID NO: 140 - protein, 15 aa, Artificial
  Description of Artificial Sequence Synthetic peptide
HNVWATHACVPTDPN

SEQ ID NO: 141 - protein, 15 aa, Artificial
  Description of Artificial Sequence Synthetic peptide
ATHACVPTDPNPQEI

SEQ ID NO: 142 - protein, 15 aa, Artificial
  Description of Artificial Sequence Synthetic peptide
CVPTDPNPQEIVLEN

SEQ ID NO: 143 - protein, 15 aa, Artificial
  Description of Artificial Sequence Synthetic peptide
DPNPQEIVLENVTEN

SEQ ID NO: 144 - protein, 15 aa, Artificial
  Description of Artificial Sequence Synthetic peptide
PQEIVLENVTENFNM

SEQ ID NO: 145 - protein, 15 aa, Artificial
  Description of Artificial Sequence Synthetic peptide
VLENVTENFNMWKNN

SEQ ID NO: 146 - protein, 15 aa, Artificial
  Description of Artificial Sequence Synthetic peptide
VTENFNMWKNNMVEQ

SEQ ID NO: 147 - protein, 15 aa, Artificial
  Description of Artificial Sequence Synthetic peptide
FNMWKNNMVEQMHED

SEQ ID NO: 148 - protein, 15 aa, Artificial
  Description of Artificial Sequence Synthetic peptide
KNNMVEQMHEDIISL

SEQ ID NO: 149 - protein, 15 aa, Artificial
  Description of Artificial Sequence Synthetic peptide
VEQMHEDIISLWDQS

SEQ ID NO: 150 - protein, 15 aa, Artificial
  Description of Artificial Sequence Synthetic peptide
HEDIISLWDQSLEPC

SEQ ID NO: 151 - protein, 15 aa, Artificial
  Description of Artificial Sequence Synthetic peptide
ISLWDQSLEPCVKLT

SEQ ID NO: 152 - protein, 15 aa, Artificial
  Description of Artificial Sequence Synthetic peptide
DQSLEPCVKLTPLCV

SEQ ID NO: 153 - protein, 15 aa, Artificial
  Description of Artificial Sequence Synthetic peptide
EPCVKLTPLCVTLHC

SEQ ID NO: 154 - protein, 15 aa, Artificial
  Description of Artificial Sequence Synthetic peptide
KLTPLCVTLHCTNLE

SEQ ID NO: 155 - protein, 15 aa, Artificial
  Description of Artificial Sequence Synthetic peptide
LCVTLHCTNLENATN

SEQ ID NO: 156 - protein, 15 aa, Artificial
  Description of Artificial Sequence Synthetic peptide
LHCTNLENATNTTSS

SEQ ID NO: 157 - protein, 15 aa, Artificial
  Description of Artificial Sequence Synthetic peptide
NLENATNTTSSNWKE

SEQ ID NO: 158 - protein, 15 aa, Artificial
  Description of Artificial Sequence Synthetic peptide
ATNTTSSNWKEMNRG

SEQ ID NO: 159 - protein, 15 aa, Artificial
  Description of Artificial Sequence Synthetic peptide
TSSNWKEMNRGEIKN

SEQ ID NO: 160 - protein, 15 aa, Artificial
  Description of Artificial Sequence Synthetic peptide
WKEMNRGEIKNCSFN

SEQ ID NO: 161 - protein, 15 aa, Artificial
  Description of Artificial Sequence Synthetic peptide
NRGEIKNCSFNVTTS

SEQ ID NO: 162 - protein, 15 aa, Artificial
  Description of Artificial Sequence Synthetic peptide
IKNCSFNVTTSIGNK

SEQ ID NO: 163 - protein, 15 aa, Artificial
  Description of Artificial Sequence Synthetic peptide
SFNVTTSIGNKMQKE

SEQ ID NO: 164 - protein, 15 aa, Artificial
  Description of Artificial Sequence Synthetic peptide
TTSIGNKMQKEYALF

SEQ ID NO: 165 - protein, 15 aa, Artificial
  Description of Artificial Sequence Synthetic peptide
GNKMQKEYALFYRLD

SEQ ID NO: 166 - protein, 15 aa, Artificial
  Description of Artificial Sequence Synthetic peptide
MQKEYALFYRLDVVP

SEQ ID NO: 167 - protein, 15 aa, Artificial
  Description of Artificial Sequence Synthetic peptide
YALFYRLDVVPIDND

SEQ ID NO: 168 - protein, 15 aa, Artificial
  Description of Artificial Sequence Synthetic peptide
YRLDVVPIDNDNTSY

SEQ ID NO: 169 - protein, 15 aa, Artificial
  Description of Artificial Sequence Synthetic peptide
VVPIDNDNTSYNLIN

SEQ ID NO: 170 - protein, 15 aa, Artificial
  Description of Artificial Sequence Synthetic peptide
DNDNTSYNLINCNTS

SEQ ID NO: 171 - protein, 15 aa, Artificial
  Description of Artificial Sequence Synthetic peptide
TSYNLINCNTSVITQ

SEQ ID NO: 172 - protein, 15 aa, Artificial
  Description of Artificial Sequence Synthetic peptide
LINCNTSVITQACPK

SEQ ID NO: 173 - protein, 15 aa, Artificial
  Description of Artificial Sequence Synthetic peptide
NTSVITQACPKVSFE

SEQ ID NO: 174 - protein, 15 aa, Artificial
  Description of Artificial Sequence Synthetic peptide
ITQACPKVSFEPIPI

SEQ ID NO: 175 - protein, 15 aa, Artificial
  Description of Artificial Sequence Synthetic peptide
CPKVSFEPIPIHYCA

SEQ ID NO: 176 - protein, 15 aa, Artificial
  Description of Artificial Sequence Synthetic peptide
SFEPIPIHYCAPAGF

SEQ ID NO: 177 - protein, 15 aa, Artificial
  Description of Artificial Sequence Synthetic peptide
IPIHYCAPAGFAILK

SEQ ID NO: 178 - protein, 15 aa, Artificial
  Description of Artificial Sequence Synthetic peptide
YCAPAGFAILKCNDK

SEQ ID NO: 179 - protein, 15 aa, Artificial
  Description of Artificial Sequence Synthetic peptide
AGFAILKCNDKKFNG

SEQ ID NO: 180 - protein, 15 aa, Artificial
  Description of Artificial Sequence Synthetic peptide
ILKCNDKKFNGSGPC

SEQ ID NO: 181 - protein, 15 aa, Artificial
  Description of Artificial Sequence Synthetic peptide
NDKKFNGSGPCINVS

SEQ ID NO: 182 - protein, 15 aa, Artificial
  Description of Artificial Sequence Synthetic peptide
FNGSGPCINVSTVQC

SEQ ID NO: 183 - protein, 15 aa, Artificial
  Description of Artificial Sequence Synthetic peptide
GPCINVSTVQCTHGI

SEQ ID NO: 184 - protein, 15 aa, Artificial
  Description of Artificial Sequence Synthetic peptide
NVSTVQCTHGIRPVV

SEQ ID NO: 185 - protein, 15 aa, Artificial
  Description of Artificial Sequence Synthetic peptide
VQCTHGIRPVVSTQL

SEQ ID NO: 186 - protein, 15 aa, Artificial
  Description of Artificial Sequence Synthetic peptide
HGIRPVVSTQLLLNG

SEQ ID NO: 187 - protein, 15 aa, Artificial
  Description of Artificial Sequence Synthetic peptide
PVVSTQLLLNGSLAE

SEQ ID NO: 188 - protein, 15 aa, Artificial
  Description of Artificial Sequence Synthetic peptide
TQLLLNGSLAEEGVV

SEQ ID NO: 189 - protein, 15 aa, Artificial
  Description of Artificial Sequence Synthetic peptide
LNGSLAEEGVVIRSE

SEQ ID NO: 190 - protein, 15 aa, Artificial
  Description of Artificial Sequence Synthetic peptide
LAEEGVVIRSENFTD

SEQ ID NO: 191 - protein, 15 aa, Artificial
  Description of Artificial Sequence Synthetic peptide
GVVIRSENFTDNVKT

SEQ ID NO: 192 - protein, 15 aa, Artificial
  Description of Artificial Sequence Synthetic peptide
RSENFTDNVKTIIVQ

SEQ ID NO: 193 - protein, 15 aa, Artificial
  Description of Artificial Sequence Synthetic peptide
FTDNVKTIIVQLKES

SEQ ID NO: 194 - protein, 15 aa, Artificial
  Description of Artificial Sequence Synthetic peptide
VKTIIVQLKESVEIN

SEQ ID NO: 195 - protein, 15 aa, Artificial
  Description of Artificial Sequence Synthetic peptide
IVQLKESVEINCTRP

SEQ ID NO: 196 - protein, 15 aa, Artificial
  Description of Artificial Sequence Synthetic peptide
KESVEINCTRPNNNT

SEQ ID NO: 197 - protein, 15 aa, Artificial
  Description of Artificial Sequence Synthetic peptide
EINCTRPNNNTRKSI

SEQ ID NO: 198 - protein, 15 aa, Artificial
  Description of Artificial Sequence Synthetic peptide
TRPNNNTRKSIPIGP

SEQ ID NO: 199 - protein, 15 aa, Artificial
  Description of Artificial Sequence Synthetic peptide
NNTRKSIPIGPGKAF

SEQ ID NO: 200 - protein, 15 aa, Artificial
  Description of Artificial Sequence Synthetic peptide
KSIPIGPGKAFYATG

SEQ ID NO: 201 - protein, 15 aa, Artificial
  Description of Artificial Sequence Synthetic peptide
IGPGKAFYATGDIIG

SEQ ID NO: 202 - protein, 15 aa, Artificial
  Description of Artificial Sequence Synthetic peptide
KAFYATGDIIGDIRQ

SEQ ID NO: 203 - protein, 15 aa, Artificial
  Description of Artificial Sequence Synthetic peptide
ATGDIIGDIRQAHCN

SEQ ID NO: 204 - protein, 15 aa, Artificial
  Description of Artificial Sequence Synthetic peptide
IIGDIRQAHCNISGE

SEQ ID NO: 205 - protein, 15 aa, Artificial
  Description of Artificial Sequence Synthetic peptide
IRQAHCNISGEKWNN

SEQ ID NO: 206 - protein, 15 aa, Artificial
  Description of Artificial Sequence Synthetic peptide
HCNISGEKWNNTLKQ

SEQ ID NO: 207 - protein, 15 aa, Artificial
  Description of Artificial Sequence Synthetic peptide
SGEKWNNTLKQIVTK

SEQ ID NO: 208 - protein, 15 aa, Artificial
  Description of Artificial Sequence Synthetic peptide
WNNTLKQIVTKLQAQ

SEQ ID NO: 209 - protein, 15 aa, Artificial
  Description of Artificial Sequence Synthetic peptide
LKQIVTKLQAQFENK

SEQ ID NO: 210 - protein, 15 aa, Artificial
  Description of Artificial Sequence Synthetic peptide
VTKLQAQFENKTIVF

SEQ ID NO: 211 - protein, 15 aa, Artificial
  Description of Artificial Sequence Synthetic peptide
LQAQFENKTIVFKQS

SEQ ID NO: 212 - protein, 15 aa, Artificial
  Description of Artificial Sequence Synthetic peptide
FENKTIVFKQSSGGD

SEQ ID NO: 213 - protein, 15 aa, Artificial
  Description of Artificial Sequence Synthetic peptide
TIVFKQSSGGDPEIV

SEQ ID NO: 214 - protein, 15 aa, Artificial
  Description of Artificial Sequence Synthetic peptide
KQSSGGDPEIVMHSF

SEQ ID NO: 215 - protein, 15 aa, Artificial
  Description of Artificial Sequence Synthetic peptide
GGDPEIVMHSFNCGG

SEQ ID NO: 216 - protein, 15 aa, Artificial
  Description of Artificial Sequence Synthetic peptide
EIVMHSFNCGGEFFY

SEQ ID NO: 217 - protein, 15 aa, Artificial
  Description of Artificial Sequence Synthetic peptide
HSFNCGGEFFYCNST

SEQ ID NO: 218 - protein, 15 aa, Artificial
  Description of Artificial Sequence Synthetic peptide
CGGEFFYCNSTQLFN

SEQ ID NO: 219 - protein, 15 aa, Artificial
  Description of Artificial Sequence Synthetic peptide
FFYCNSTQLFNSTWN

SEQ ID NO: 220 - protein, 15 aa, Artificial
  Description of Artificial Sequence Synthetic peptide
NSTQLFNSTWNNTIG

SEQ ID NO: 221 - protein, 15 aa, Artificial
  Description of Artificial Sequence Synthetic peptide
LFNSTWNNTIGPNNT

SEQ ID NO: 222 - protein, 15 aa, Artificial
  Description of Artificial Sequence Synthetic peptide
TWNNTIGPNNTNGTI

SEQ ID NO: 223 - protein, 15 aa, Artificial
  Description of Artificial Sequence Synthetic peptide
TIGPNNTNGTITLPC

SEQ ID NO: 224 - protein, 15 aa, Artificial
  Description of Artificial Sequence Synthetic peptide
NNTNGTITLPCRIKQ

SEQ ID NO: 225 - protein, 15 aa, Artificial
  Description of Artificial Sequence Synthetic peptide
GTITLPCRIKQIINR

SEQ ID NO: 226 - protein, 15 aa, Artificial
  Description of Artificial Sequence Synthetic peptide
LPCRIKQIINRWQEV

SEQ ID NO: 227 - protein, 15 aa, Artificial
  Description of Artificial Sequence Synthetic peptide
IKQIINRWQEVGKAM

SEQ ID NO: 228 - protein, 15 aa, Artificial
  Description of Artificial Sequence Synthetic peptide
INRWQEVGKAMYAPP

SEQ ID NO: 229 - protein, 15 aa, Artificial
  Description of Artificial Sequence Synthetic peptide
WQEVGKAMYAPPIRG

SEQ ID NO: 230 - protein, 15 aa, Artificial
  Description of Artificial Sequence Synthetic peptide
GKAMYAPPIRGQIRC

SEQ ID NO: 231 - protein, 15 aa, Artificial
  Description of Artificial Sequence Synthetic peptide
YAPPIRGQIRCSSNI

SEQ ID NO: 232 - protein, 15 aa, Artificial
  Description of Artificial Sequence Synthetic peptide
IRGQIRCSSNITGLL

SEQ ID NO: 233 - protein, 15 aa, Artificial
  Description of Artificial Sequence Synthetic peptide
IRCSSNITGLLLTRD

SEQ ID NO: 234 - protein, 15 aa, Artificial
  Description of Artificial Sequence Synthetic peptide
SNITGLLLTRDGGRE

SEQ ID NO: 235 - protein, 15 aa, Artificial
  Description of Artificial Sequence Synthetic peptide
GLLLTRDGGREVGNT

SEQ ID NO: 236 - protein, 15 aa, Artificial
  Description of Artificial Sequence Synthetic peptide
TRDGGREVGNTTEIF

SEQ ID NO: 237 - protein, 15 aa, Artificial
  Description of Artificial Sequence Synthetic peptide
GREVGNTTEIFRPGG

SEQ ID NO: 238 - protein, 15 aa, Artificial
  Description of Artificial Sequence Synthetic peptide
GNTTEIFRPGGGDMR

SEQ ID NO: 239 - protein, 15 aa, Artificial
  Description of Artificial Sequence Synthetic peptide
EIFRPGGGDMRDNWR

SEQ ID NO: 240 - protein, 15 aa, Artificial
  Description of Artificial Sequence Synthetic peptide
PGGGDMRDNWRSELY

SEQ ID NO: 241 - protein, 15 aa, Artificial
  Description of Artificial Sequence Synthetic peptide
DMRDNWRSELYKYKV

SEQ ID NO: 242 - protein, 15 aa, Artificial
  Description of Artificial Sequence Synthetic peptide
NWRSELYKYKVVKIE

SEQ ID NO: 243 - protein, 15 aa, Artificial
  Description of Artificial Sequence Synthetic peptide
ELYKYKVVKIEPLGV

SEQ ID NO: 244 - protein, 15 aa, Artificial
  Description of Artificial Sequence Synthetic peptide
YKVVKIEPLGVAPTK

SEQ ID NO: 245 - protein, 15 aa, Artificial
  Description of Artificial Sequence Synthetic peptide
KIEPLGVAPTKAKRR

SEQ ID NO: 246 - protein, 15 aa, Artificial
  Description of Artificial Sequence Synthetic peptide
LGVAPTKAKRRVVQR

SEQ ID NO: 247 - protein, 15 aa, Artificial
  Description of Artificial Sequence Synthetic peptide
PTKAKRRVVQREKRA

SEQ ID NO: 248 - protein, 15 aa, Artificial
  Description of Artificial Sequence Synthetic peptide
KRRVVQREKRAVTLG

SEQ ID NO: 249 - protein, 15 aa, Artificial
  Description of Artificial Sequence Synthetic peptide
VQREKRAVTLGAVFL

SEQ ID NO: 250 - protein, 15 aa, Artificial
  Description of Artificial Sequence Synthetic peptide
KRAVTLGAVFLGFLG

SEQ ID NO: 251 - protein, 15 aa, Artificial
  Description of Artificial Sequence Synthetic peptide
TLGAVFLGFLGAAGS

SEQ ID NO: 252 - protein, 15 aa, Artificial
  Description of Artificial Sequence Synthetic peptide
VFLGFLGAAGSTMGA

SEQ ID NO: 253 - protein, 15 aa, Artificial
  Description of Artificial Sequence Synthetic peptide
FLGAAGSTMGAASLT

SEQ ID NO: 254 - protein, 15 aa, Artificial
  Description of Artificial Sequence Synthetic peptide
AGSTMGAASLTLTVQ

SEQ ID NO: 255 - protein, 15 aa, Artificial
  Description of Artificial Sequence Synthetic peptide
MGAASLTLTVQARQL

SEQ ID NO: 256 - protein, 15 aa, Artificial
  Description of Artificial Sequence Synthetic peptide
SLTLTVQARQLLSGI

SEQ ID NO: 257 - protein, 15 aa, Artificial
  Description of Artificial Sequence Synthetic peptide
LWVTVYYGVPVWKEA

SEQ ID NO: 258 - protein, 15 aa, Artificial
  Description of Artificial Sequence Synthetic peptide
VYYGVPVWKEATTTL

SEQ ID NO: 259 - protein, 15 aa, Artificial
  Description of Artificial Sequence Synthetic peptide
VPVWKEATTTLFCAS

SEQ ID NO: 260 - protein, 15 aa, Artificial
  Description of Artificial Sequence Synthetic peptide
EDIISLWDQSLKPCV

SEQ ID NO: 261 - protein, 15 aa, Artificial
  Description of Artificial Sequence Synthetic peptide
SLWDQSLKPCVKLTP

SEQ ID NO: 262 - protein, 15 aa, Artificial
  Description of Artificial Sequence Synthetic peptide
QSLKPCVKLTPLCVT

SEQ ID NO: 263 - protein, 15 aa, Artificial
  Description of Artificial Sequence Synthetic peptide
QKEYALFYKLDVVPI

SEQ ID NO: 264 - protein, 15 aa, Artificial
  Description of Artificial Sequence Synthetic peptide
ALFYKLDVVPIDNDN

SEQ ID NO: 265 - protein, 15 aa, Artificial
  Description of Artificial Sequence Synthetic peptide
GPCTNVSTVQCTHGI

SEQ ID NO: 266 - protein, 502 aa, Human immunodeficiency virus type 1
MGARASVLSGGELDKWEKIRLRPGGKKKYKLKHIVWASRELERFAVNPGLLETSEGCRQILGQLQPSLQTGSEELRSLYNTVATLYCVHQRIDVKDTKEALEKIEEEQNKSKKKAQQAAAAAGTGNSSQVSQNYPIVQNLQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQDVKNWMTETLLVQNANPDCKTILKALGPAATLEEMMTACQGVGGPGHKARVLAEAMSQVTNPANIMMQRGNFRNQRKTVKCFNCGKEGHIAKNCRAPRKKGCWRCGREGHQMKDCTERQANFLGKIWPSYKGRPGNFLQSRPEPTAPPEESFRFGEEKTTPSQKQEPIDKELYPLTSLRSLFGNDPSSQ

SEQ ID NO: 267 - protein, 487 aa, Human immunodeficiency virus type 1
MRVKGIRKNYQHLWRGGTLLLGMLMICSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGV

SEQ ID NO: 268 - protein, 8 aa, Human immunodeficiency virus type 1
SIINFEKL

SEQ ID NO: 269 - protein, 8 aa, Human immunodeficiency virus type 1
KVVRFDKL

SEQ ID NO: 270 - protein, 8 aa, Human immunodeficiency virus type 1
CFDVFKEL

SEQ ID NO: 271 - DNA, 24 bp, Artificial
  Description of Artificial Sequence Synthetic primer
ggggaattcatggagccagtagat

SEQ ID NO: 272 - DNA, 24 bp, Artificial
  Description of Artificial Sequence Synthetic primer
caagaattcctattccttcgggcc

SEQ ID NO: 273 - DNA, 20 bp, Artificial
  Description of Artificial Sequence Synthetic primer
cgagctgcaagaactcttcc

SEQ ID NO: 274 - DNA, 20 bp, Artificial
  Description of Artificial Sequence Synthetic primer
aggccttccatctgttgctg

SEQ ID NO: 275 - DNA, 21 bp, Artificial
  Description of Artificial Sequence Synthetic primer
gaagcatccaggaagtcagcc

SEQ ID NO: 276 - DNA, 21 bp, Artificial
  Description of Artificial Sequence Synthetic primer
accttcttcttctattccggg

SEQ ID NO: 277 - DNA, 30 bp, Artificial
  Description of Artificial Sequence Synthetic primer
tgacggggtcacccacactgtgcccatcta

SEQ ID NO: 278 - DNA, 30 bp, Artificial
  Description of Artificial Sequence Synthetic primer
agtcatagtccgcctagaagcatttgcggt

SEQ ID NO: 279 - protein, 14 aa, Artificial
  Description of Artificial Sequence Synthetic peptide
CLGGLLTMVAGAVW

SEQ ID NO: 280 - protein, 9 aa, Artificial
  Description of Artificial Sequence Synthetic peptide
IVTDFSVIK

SEQ ID NO: 281 - protein, 10 aa, Artificial
  Description of Artificial Sequence Synthetic peptide
AVFSRKSDAK

SEQ ID NO: 282 - protein, 9 aa, Artificial
  Description of Artificial Sequence Synthetic peptide
CLGGLLTMV

SEQ ID NO: 283 - protein, 9 aa, Artificial
  Description of Artificial Sequence Synthetic peptide
YLQQNWWTL

SEQ ID NO: 284 - protein, 86 aa, Human immunodeficiency virus
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 285 - protein, 40 aa, Human immunodeficiency virus
YGRKKRRQRRRPPQGSQTHQVSLSKQPTSQSRGDPTGPKE

SEQ ID NO: 286 - protein, 86 aa, Artificial
  Description of Artificial Sequence Synthetic peptide
MEPVDPRLEPWKHPGSQPKTAGTNCYCKKCCFHCQVCFITKALGISYGRKKRRQRRRPPQGSQTHQVSLSKQPTSQSRGDPTGPKE

SEQ ID NO: 287 - protein, 847 aa, Human immunodeficiency virus type 1
MRVKGIRKNYQHLWRGGTLLLGMLMICSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKRAVTLGAMFLGFLGAAGSTMGARSLTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYIKIFIMIVGGLVGLRIVFTVLSIVNRVRQGYSPLSFQTRFPAPRGPDRPEGIEEEGGERDRDRSSPLVHGLLALIWDDLRSLCLFSYHRLRDLILIAARIVELLGRRGWEALKYWGNLLQYWIQELKNSAVSLFDAIAIAVAEGTDRIIEVAQRIGRAFLHIPRRIRQGFERALL

SEQ ID NO: 288 - protein, 502 aa, Human immunodeficiency virus type 1
MRVKGIRKNYQHLWRGGTLLLGMLMICSAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVTTSIRNKMQKEYALFYKLDVVPIDNDNTSYKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAKRRVVQREKR

SEQ ID NO: 289 - protein, 4 aa, Artificial
  Description of Artificial Sequence Synthetic peptide
LLVY

SEQ ID NO: 290 - protein, 4 aa, Artificial
  Description of Artificial Sequence Synthetic peptide
YVAD